(19) United States
(12) Patent Application Publication
(10) Pub. No.: US 2025/0109130 A1
(43) Pub. Date: Apr. 3, 2025

(54) ISOTOPICALLY ENRICHED ANALOGS OF 2-BROMO-LSD, LSD, ALD-52, AND 1P-LSD

(71) Applicant: Terran Biosciences Inc., Miami Beach, FL (US)

(72) Inventors: Matthew DUNCTON, Las Vegas, NV (US); Samuel CLARK, Miami, FL (US)

(21) Appl. No.: 18/719,948

(22) PCT Filed: Dec. 16, 2022
(86) PCT No.: PCT/US2022/081816

§ 371 Date: Jun. 14, 2024

Related U.S. Application Data
(60) Provisional application No. 63/303,895, filed on Jan. 27, 2022.
(60) Provisional application No. 63/291,388, filed on Dec. 18, 2021.
(60) Provisional application No. 63/290,562, filed on Dec. 16, 2021.

(51) Int. Cl. C07D 457/06; A61K 31/36; A61K 31/48; A61K 31/517; A61P 25/28; A61P 25/32

(52) U.S. Cl. CPC C07D 457/06; A61K 31/36; A61K 31/48; A61K 31/517; A61P 25/28; A61P 25/32

(57) ABSTRACT

Disclosed herein are isotopically enriched compounds of 2-bromolysergic acid diethylamide, LSD, ALD-52, and 1P-LSD, as well as methods for their use in treating neurologic and brain disorders.

### CROSS REFERENCE TO RELATED APPLICATIONS

The present application claims priority to and the benefit of U.S. Provisional Patent Application Nos. 63/290,562, filed Dec. 16, 2021, 63/291,388, filed Dec. 18, 2021, and 63/303,895, filed Jan. 27, 2022, which are incorporated by reference in their entireties for all purposes.

### FIELD

The present disclosure relates to isotopically enriched analogs of 2-bromolysergic acid diethylamide (2-Br-LSD or 2-bromo-LSD), lysergic acid diethylamide (LSD), acetyl lysergic acid diethylamide (1-acetyl-LSD or ALD-52), and 1-propionyl lysergic acid diethylamide (1P-LSD), as well as their use to treat brain and neurological disorders. The disclosure further relates to the provision of isotopically enriched compounds with improved characteristics.

### BACKGROUND

Major depressive disorder and related neuropsychiatric diseases are among the leading causes of disability worldwide. Despite recent advances, there remains a need for new therapeutics to support treatment of debilitating neuropsychiatric diseases. Recently, psychedelic compounds have received renewed interest for the treatment of depression and other disorders. For example, the Food and Drug Administration (FDA) recently approved the dissociative anesthetic ketamine for treatment-resistant depression, making it the first mechanistically distinct medicine to be introduced to psychiatry in nearly thirty years. Ketamine is a member of a class of compounds known as psychoplastogens. Psychoplastogens promote neuronal growth through a mechanism involving the activation of AMPA receptors, the tropomyosin receptor kinase B (TrkB), and the mammalian target of rapamycin (mTOR). As pyramidal neurons in the PFC exhibit top-down control over areas of the brain controlling motivation, fear, and reward, these effects support clinical development of psychoplastogenic compounds for their antidepressant, anxiolytic, and anti-addictive effects properties.

Certain compounds from the psychedelic class of compounds have shown promise in therapeutic applications. Disclosed herein are 2-bromolysergic acid diethylamide, LSD, ALD-52 and 1P-LSD analogs with improved properties.

### SUMMARY

The present disclosure relates to 2-bromolysergic acid diethylamide, LSD, ALD-52 and 1P-LSD analogs for the treatment of neurological and psychiatric disorders. In one embodiment the compounds have improved efficacy, improved pharmacokinetic properties or both relative to known compounds. In one embodiment the disclosed compounds are isotopically enriched at one or more position.

The foregoing and other objects, features, and advantages of the invention will become more apparent from the following detailed description.

### DETAILED DESCRIPTION

### General

Disclosed herein are 2-bromolysergic acid diethylamide (2-Br-LSD or 2-bromo-LSD), lysergic acid diethylamide (LSD), acetyl lysergic acid diethylamide (1-acetyl-LSD or ALD-52), and 1-propionyl lysergic acid diethylamide (1P-LSD) analogs, in particular, isotopically labeled analogs, or isotopologues. The presently disclosed isotopologues are useful for the treatment of a variety of brain disorders and other conditions. Without limitation to any particular theory, it is believed that the present compounds increase neuronal plasticity, and increase at least one of translation, transcription, or secretion of neurotrophic factors. Moreover, by virtue of their isotopic enrichment, the presently disclosed compounds have improved pharmacokinetic and pharmacodynamic properties as compared to previously disclosed molecules. In some embodiments the isotopic labels of the present compounds allow monitoring of its pharmacodynamic and ADME behavior following in vivo administration. In some embodiments, the isotopically enriched compounds described herein provide better therapeutic potential for neurological diseases than known compounds.

### Terms and Abbreviations

The term “isotopic enrichment factor” as used herein means the ratio between the isotopic abundance and the natural abundance of a specified isotope. It will be recognized that some variation of natural isotopic abundance occurs in a synthesized compound depending upon the origin of chemical materials used in the synthesis. Thus, a preparation of any compound will inherently contain small amounts of isotopologues, including deuterated isotopologues. The concentration of naturally abundant stable hydrogen isotopes, notwithstanding this variation, is small and immaterial as compared to the degree of stable isotopic substitution of compounds of this disclosure. In a compound of this disclosure, when a particular position is designated as having a particular isotope, such as deuterium, it is understood that the abundance of deuterium at that position is substantially greater than the natural abundance of deuterium, which is about 0.015% (on a mol/mol basis). A position designated as a particular isotope will have a minimum isotopic enrichment factor of at least 3000 (45% incorporation of the indicated isotope). Thus, isotopically enriched compounds disclosed herein having deuterium will have a minimum isotopic enrichment factor of at least 3000 (45% deuterium incorporation) at each atom designated as deuterium in the compound. Such compounds may be referred to herein as “deuterated” compounds.

In some embodiments, disclosed compounds have an isotopic enrichment factor for each designated atom of at least 3500 (52.5%). For example, for such disclosed compounds that are deuterium isotopologues, the compounds have an isotopic enrichment factor for each designated hydrogen atom of at least 3500 (52.5% deuterium incorporation at each designated atom), at least 4000 (60% deuterium incorporation), at least 4500 (67.5% deuterium incorporation), at least 5000 (75% deuterium), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation). As above, such compounds also are referred to as “deuterated” compounds.

In the compounds of this disclosure any atom not specifically designated as a particular isotope is meant to represent any stable isotope of that atom. Unless otherwise stated, when a position is designated specifically as “H”, the position is understood to have hydrogen at about its natural abundance isotopic composition.

Methyl and ethyl may be saturated with any stable isotope of hydrogen, e.g., protium, deuterium, and tritium.

The term “isotopologue” refers to a species that has the same chemical structure and formula as another compound, with the exception of the isotopic composition at one or more positions, e.g., H vs. D. Thus, isotopologues differ in their isotopic composition.

“Halo” or “halogen” refers to bromo, chloro, fluoro, or iodo. In some embodiments of the compounds disclosed herein, halogen is fluoro or chloro. In some embodiments of the compounds disclosed herein, halogen is selected from chloro, bromo and iodo, or from chloro and iodo, or from bromo and iodo.

“Salt” refers to acid or base salts of the compounds used in the methods of the present invention, in particular pharmaceutically acceptable salts. Illustrative examples of pharmaceutically acceptable salts are mineral acid (hydrochloric acid, hydrobromic acid, phosphoric acid, and the like) salts, organic acid (fumaric acid, acetic acid, propionic acid, glutamic acid, citric acid, tartaric acid and the like) salts, quaternary ammonium (methyl iodide, ethyl iodide, and the like) salts. It is understood that the pharmaceutically acceptable salts are non-toxic. Additional suitable pharmaceutically acceptable salts are known to those of skill in the art. See, e.g., Remington: The Science and Practice of Pharmacy, volume I and volume H. (22^{nd} Ed., University of the Sciences, Philadelphia)., which is incorporated herein by reference.

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

“Pharmaceutically acceptable salt” refers to a compound in salt form, wherein the salt form is suitable for administration to a subject. Representative pharmaceutically acceptable salts include salts of acetic, ascorbic, benzenesulfonic, benzoic, camphorsulfonic, citric, ethanesulfonic, edisylic, fumaric, gentisic, gluconic, glucoronic, glutamic, hippuric, hydrobromic, hydrochloric, isethionic, lactic, lactobionic, maleic, malic, mandelic, methanesulfonic, mucic, naphthalenesulfonic, naphthalene-1,5-disulfonic, naphthalene-2, 6-disulfonic, nicotinic, nitric, orotic, pamoic, pantothenic, phosphoric, succinic, sulfuric, tartaric, p-toluenesulfonic and xinafoic acid, and the like

“Pharmaceutically acceptable excipient” refers to a substance that aids the administration of an active agent to and absorption by a subject. Pharmaceutical excipients useful in the present invention include, but are not limited to, binders, fillers, disintegrants, lubricants, coatings, sweeteners, flavors and colors. one of skill in the art will recognize that other pharmaceutical excipients are useful in the present invention.

“Composition” refers to a product comprising the specified ingredients in the specified amounts, as well as any product, which results, directly or indirectly, from combination of the specified ingredients in the specified amounts. By “pharmaceutically acceptable” it is meant the carrier, diluent or excipient must be compatible with the other ingredients of the formulation.

“Isomers” refers to compounds with same chemical formula but different connectivity between the atoms in the molecule, leading to distinct chemical structures. Isomers include structural isomers and stereoisomers. Examples of structural isomers include, but are not limited to tautomers and regioisomers. Examples of stereoisomers include but are not limited to diastereomers and enantiomers.

“Administering” refers to any suitable mode of administration, including, oral administration, administration as a suppository, topical contact, parenteral, intravenous, intraperitoneal, intramuscular, intralesional, intranasal or subcutaneous administration, intrathecal administration, or the implantation of a slow-release device e.g., a mini-osmotic pump, to the subject.

“Subject” refers to an animal, such as a mammal, including, but not limited to, primates (e.g., humans), cows, sheep, goats, horses, dogs, cats, rabbits, rats, mice and the like. In some embodiments, the subject is a human subject.

“Therapeutically effective amount” or “therapeutically sufficient amount” or “effective or sufficient amount” refers to a dose that produces therapeutic effects for which it is administered.

The exact dose will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins). In sensitized cells, the therapeutically effective dose can often be lower than the conventional therapeutically effective dose for non-sensitized cells.

“Neuronal plasticity” refers to the ability of the brain to change its structure and/or function continuously throughout a subject's life. Examples of the changes to the brain include, but are not limited to, the ability to adapt or respond to internal and/or external stimuli, such as due to an injury, and the ability to produce new neurites, dendritic spines, and synapses.

“Brain disorder” refers to a neurological disorder which affects the brain's structure and function. Brain disorders can include, but are not limited to, Alzheimer's, Parkinson's disease, psychological disorder, depression, treatment resistant depression, addiction, anxiety, post-traumatic stress disorder, suicidal ideation, major depressive disorder, bipolar disorder, schizophrenia, stroke, traumatic brain injury, and substance use disorder.

“Combination therapy” refers to a method of treating a disease or disorder, wherein two or more different pharmaceutical agents are administered in overlapping regimens so that the subject is simultaneously exposed to both agents. For example, the compounds of the invention can be used in combination with other pharmaceutically active compounds. The compounds of the invention can be administered simultaneously (as a single preparation or separate preparation) or sequentially to the other drug therapy. In general, a combination therapy envisions administration of two or more drugs during a single cycle or course of therapy.

“Neurotrophic factors” refers to a family of soluble peptides or proteins which support the survival, growth, and differentiation of developing and mature neurons.

“Modulate” or “modulating” or “modulation” refers to an increase or decrease in the amount, quality, or effect of a particular activity, function or molecule. By way of illustration and not limitation, agonists, partial agonists, antagonists, and allosteric modulators (e.g., a positive allosteric modulator) of a G protein-coupled receptor (e.g., 5HT_{2A}) are modulators of the receptor.

“Agonism” refers to the activation of a receptor or enzyme by a modulator, or agonist, to produce a biological response.

“Agonist” refers to a modulator that binds to a receptor or enzyme and activates the receptor to produce a biological response. By way of example only, “5HT_{2A} agonist” can be used to refer to a compound that exhibits an EC₅₀ with respect to 5HT_{2A} activity of no more than about 100 mM. In some embodiments, the term “agonist” includes full agonists or partial agonists. “Full agonist” refers to a modulator that binds to and activates a receptor with the maximum response that an agonist can elicit at the receptor. “Partial agonist” refers to a modulator that binds to and activates a given receptor, but has partial efficacy, that is, less than the maximal response, at the receptor relative to a full agonist.

“Positive allosteric modulator” refers to a modulator that binds to a site distinct from the orthosteric binding site and enhances or amplifies the effect of an agonist.

“Antagonism” refers to the inactivation of a receptor or enzyme by a modulator, or antagonist. Antagonism of a receptor, for example, is when a molecule binds to the receptor and does not allow activity to occur.

“Antagonist” or “neutral antagonist” refers to a modulator that binds to a receptor or enzyme and blocks a biological response. An antagonist has no activity in the absence of an agonist or inverse agonist but can block the activity of either, causing no change in the biological response.

As used herein, “alkyl”, “C₁, C₂, C₃, C₄, C₅ or C₆ alkyl” or “C₁-C₆ alkyl” is intended to include C₁, C₂, C₃, C₄, C₅ or C₆ straight chain (linear) saturated aliphatic hydrocarbon groups and C₃, C₄, C₅ or C₆ branched saturated aliphatic hydrocarbon groups. For example, C₁-C₆ alkyl is intends to include C₁, C₂, C₃, C₄, C₅ and C₆ alkyl groups. Alkyl groups (e.g., methyl) may be saturated with any stable isotope of hydrogen, e.g., protium, deuterium, and tritium. Examples of alkyl include, moieties having from one to six carbon atoms, such as, but not limited to, methyl, ethyl, n-propyl, i-propyl, n-butyl, s-butyl, t-butyl, n-pentyl, i-pentyl, or n-hexyl. In some embodiments, a straight chain or branched alkyl has six or fewer carbon atoms (e.g., C₁-C₆ for straight chain, C₃-C₆ for branched chain), and in another embodiment, a straight chain or branched alkyl has four or fewer carbon atoms.

As used herein, the term “optionally substituted alkyl” refers to unsubstituted alkyl or alkyl having designated substituents replacing one or more hydrogen, deuterium, or tritium atoms on one or more carbons of the hydrocarbon backbone. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

As used herein, the term “alkenyl” includes unsaturated aliphatic groups analogous in length and possible substitution to the alkyls described above, but that contain at least one double bond. For example, the term “alkenyl” includes straight chain alkenyl groups (e.g., ethenyl, propenyl, butenyl, pentenyl, hexenyl, heptenyl, octenyl, nonenyl, decenyl), and branched alkenyl groups. In some embodiments, a straight chain or branched alkenyl group has six or fewer carbon atoms in its backbone (e.g., C₂-C₆ for straight chain, C₃-C₆ for branched chain). The term “C₂-C₆” includes alkenyl groups containing two to six carbon atoms. The term “C₃-C₆” includes alkenyl groups containing three to six carbon atoms.

As used herein, the term “optionally substituted alkenyl” refers to unsubstituted alkenyl or alkenyl having designated substituents replacing one or more hydrogen atoms on one or more hydrocarbon backbone carbon atoms. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, heterocyclyl, alkylaryl, or an aromatic or heteroaromatic moiety.

As used herein, the term “alkynyl” includes unsaturated aliphatic groups analogous in length and possible substitution to the alkyls described above, but which contain at least one triple bond. For example, “alkynyl” includes straight chain alkynyl groups (e.g., ethynyl, propynyl, butynyl, pentynyl, hexynyl, heptynyl, octynyl, nonynyl, decynyl), and branched alkynyl groups. In some embodiments, a straight chain or branched alkynyl group has six or fewer carbon atoms in its backbone (e.g., C₂-C₆ for straight chain, C₃-C₆ for branched chain). The term “C₂-C₆” includes alkynyl groups containing two to six carbon atoms. The term “C₃-C₆” includes alkynyl groups containing three to six carbon atoms. As used herein, “C₂-C₆ alkenylene linker” or “C₂-C₆ alkynylene linker” is intended to include C₂, C₃, C₄, C₅ or C₆ chain (linear or branched) divalent unsaturated aliphatic hydrocarbon groups. For example, C₂-C₆ alkenylene linker is intended to include C₂, C₃, C₄, C₅ and C₆ alkenylene linker groups.

As used herein, the term “optionally substituted alkynyl” refers to unsubstituted alkynyl or alkynyl having designated substituents replacing one or more hydrogen atoms on one or more hydrocarbon backbone carbon atoms. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

Other optionally substituted moieties (such as optionally substituted cycloalkyl, heterocycloalkyl, aryl, or heteroaryl) include both the unsubstituted moieties and the moieties having one or more of the designated substituents. For example, substituted heterocycloalkyl includes those substituted with one or more alkyl groups, such as 2,2,6,6-tetramethyl-piperidinyl and 2,2,6,6-tetramethyl-1,2,3,6-tetrahydropyridinyl.

As used herein, the term “cycloalkyl” refers to a saturated or partially unsaturated hydrocarbon monocyclic or polycyclic (e.g., fused, bridged, or spiro rings) system having 3 to 30 carbon atoms (e.g., C₃-C₁₂, C₃-C₁₀, or C₃-C₅). Examples of cycloalkyl include, but are not limited to, cyclopropyl, cyclobutyl, cyclopentyl, cyclohexyl, cycloheptyl, cyclooctyl, cyclopentenyl, cyclohexenyl, cycloheptenyl, 1,2,3,4-tetrahydronaphthalenyl, and adamantyl. In the case of polycyclic cycloalkyl, only one of the rings in the cycloalkyl needs to be non-aromatic.

As used herein, the term “heterocycloalkyl” refers to a saturated or partially unsaturated 3-8 membered monocyclic, 7-12 membered bicyclic (fused, bridged, or spiro rings), or 11-14 membered tricyclic ring system (fused, bridged, or spiro rings) having one or more heteroatoms (such as O, N, S, P, or Se), e.g., 1 or 1-2 or 1-3 or 1-4 or 1-5 or 1-6 heteroatoms, or e.g., 1, 2, 3, 4, 5, or 6 heteroatoms, independently selected from the group consisting of nitrogen, oxygen and sulfur, unless specified otherwise. Examples of heterocycloalkyl groups include, but are not limited to, piperidinyl, piperazinyl, pyrrolidinyl, dioxanyl, tetrahydrofuranyl, isoindolinyl, indolinyl, imidazolidinyl, pyrazolidinyl, oxazolidinyl, isoxazolidinyl, triazolidinyl, oxiranyl, azetidinyl, oxetanyl, thietanyl, 1,2,3,6-tetrahydropyridinyl, tetrahydropyranyl, dihydropyranyl, pyranyl, morpholinyl, tetrahydrothiopyranyl, 1,4-diazepanyl, 1,4-oxazepanyl, 2-oxa-5-azabicyclo[2.2.1]heptanyl, 2,5-diazabicyclo[2.2.1]heptanyl, 2-oxa-6-azaspiro[3.3]heptanyl, 2,6-diazaspiro[3.3]heptanyl, 1,4-dioxa-8-azaspiro[4.5]decanyl, 1,4-dioxaspiro[4.5]decanyl, 1-oxaspiro[4.5]decanyl, 1-azaspiro[4.5]decanyl, 3′H-spiro[cyclohexane-1,1′-isobenzofuran]-yl, 7′H-spiro[cyclohexane-1,5′-furo[3,4-b]pyridin]-yl, 3′H-spiro[cyclohexane-1,1′-furo[3,4-c]pyridin]-yl, 3-azabicyclo[3.1.0]hexanyl, 3-azabicyclo[3.1.0]hexan-3-yl, 1,4,5,6-tetrahydropyrrolo[3,4-c]pyrazolyl, 3,4,5,6,7,8-hexahydropyrido[4,3-d]pyrimidinyl, 4,5,6,7-tetrahydro-1H-pyrazolo[3,4-c]pyridinyl, 5,6,7,8-tetrahydropyrido[4,3-d]pyrimidinyl, 2-azaspiro[3.3]heptanyl, 2-methyl-2-azaspiro[3.3]heptanyl, 2-azaspiro[3.5]nonanyl, 2-methyl-2-azaspiro[3.5]nonanyl, 2-azaspiro[4.5]decanyl, 2-methyl-2-azaspiro[4.5]decanyl, 2-oxa-azaspiro[3.4]octanyl, 2-oxa-azaspiro[3.4]octan-6-yl, 5,6-dihydro-4H-cyclopenta[b]thiophenyl, and the like. In the case of multicyclic heterocycloalkyl, only one of the rings in the heterocycloalkyl needs to be non-aromatic (e.g., 4,5,6,7-tetrahydrobenzo[c]isoxazolyl).

It is understood that when a variable has two attachments to the rest of the formula of the compound, the two attachments could be at the same atom or different atoms of the variable. For example, when a variable (e.g., variable X) is cycloalkyl or heterocycloalkyl, and has two attachments to the rest of the formula of the compound, the two attachments could be at the same atom or different atoms of the cycloalkyl or heterocycloalkyl.

As used herein, the term “aryl” includes groups with aromaticity, including “conjugated,” or multicyclic systems with one or more aromatic rings and do not contain any heteroatom in the ring structure. The term aryl includes both monovalent species and divalent species. Examples of aryl groups include, but are not limited to, phenyl, biphenyl, naphthyl and the like. For example, an aryl is phenyl.

As used herein, the term “heteroaryl” is intended to include a stable 5-, 6-, or 7-membered monocyclic or 7-, 8-, 9-, 10-, 11- or 12-membered bicyclic aromatic heterocyclic ring which consists of carbon atoms and one or more heteroatoms, e.g., 1 or 1-2 or 1-3 or 1-4 or 1-5 or 1-6 heteroatoms, or e.g., 1, 2, 3, 4, 5, or 6 heteroatoms, independently selected from the group consisting of nitrogen, oxygen and sulfur. The nitrogen atom may be substituted or unsubstituted (i.e., N or NR wherein R is H or other substituents, as defined). The nitrogen and sulfur heteroatoms may optionally be oxidised (i.e., N→O and S(O)ₚ, where p=1 or 2). It is to be noted that total number of S and O atoms in the aromatic heterocycle is not more than 1.

Examples of heteroaryl groups include pyrrole, furan, thiophene, thiazole, isothiazole, imidazole, triazole, tetrazole, pyrazole, oxazole, isoxazole, isothiazole, pyridine, pyrazine, pyridazine, pyrimidine, and the like. Heteroaryl groups can also be fused or bridged with alicyclic or heterocyclic rings, which are not aromatic so as to form a multicyclic system (e.g., 4,5,6,7-tetrahydrobenzo[c]isoxazolyl). In some embodiments, the heteroaryl is thiophenyl or benzothiophenyl. In some embodiments, the heteroaryl is thiophenyl. In some embodiments, the heteroaryl benzothiophenyl.

Furthermore, the terms “aryl” and “heteroaryl” include multicyclic aryl and heteroaryl groups, e.g., tricyclic, bicyclic, e.g., naphthalene, benzoxazole, benzodioxazole, benzothiazole, benzoimidazole, benzothiophene, quinoline, isoquinoline, naphthrydine, indole, benzofuran, purine, benzofuran, deazapurine, indolizine.

The cycloalkyl, heterocycloalkyl, aryl, or heteroaryl ring can be substituted at one or more ring positions (e.g., the ring-forming carbon or heteroatom such as N) with such substituents as described above, for example, alkyl, alkenyl, alkynyl, halogen, hydroxyl, alkoxy, alkylcarbonyloxy, arylcarbonyloxy, alkoxycarbonyloxy, aryloxycarbonyloxy, carboxylate, alkylcarbonyl, alkylaminocarbonyl, aralkylaminocarbonyl, alkenylaminocarbonyl, alkylcarbonyl, arylcarbonyl, aralkylcarbonyl, alkenylcarbonyl, alkoxycarbonyl, aminocarbonyl, alkylthiocarbonyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety. Aryl and heteroaryl groups can also be fused or bridged with alicyclic or heterocyclic rings, which are not aromatic so as to form a multicyclic system (e.g., tetralin, methylenedioxyphenyl such as benzo[d][1,3]dioxole-5-yl).

As used herein, the term “substituted,” means that any one or more hydrogen atoms on the designated atom is replaced with a selection from the indicated groups, provided that the designated atom's normal valency is not exceeded, and that the substitution results in a stable compound. When a substituent is oxo or keto (i.e., ═O), then 2 hydrogen atoms on the atom are replaced. Keto substituents are not present on aromatic moieties. Ring double bonds, as used herein, are double bonds that are formed between two adjacent ring atoms (e.g., C═C, C═N or N═N). “Stable compound” and “stable structure” are meant to indicate a compound that is sufficiently robust to survive isolation to a useful degree of purity from a reaction mixture, and formulation into an efficacious therapeutic agent.

When a bond to a substituent is shown to cross a bond connecting two atoms in a ring, then such substituent may be bonded to any atom in the ring. When a substituent is listed without indicating the atom via which such substituent is bonded to the rest of the compound of a given formula, then such substituent may be bonded via any atom in such formula. Combinations of substituents and/or variables are permissible, but only if such combinations result in stable compounds.

When any variable (e.g., R) occurs more than one time in any constituent or formula for a compound, its definition at each occurrence is independent of its definition at every other occurrence. Thus, for example, if a group is shown to be substituted with 0-2 R moieties, then the group may optionally be substituted with up to two R moieties and R at each occurrence is selected independently from the definition of R. Also, combinations of substituents and/or variables are permissible, but only if such combinations result in stable compounds.

As used herein, the term “hydroxy” or “hydroxyl” includes groups with an —OH or —O⁻. The term “haloalkyl” or “haloalkoxyl” refers to an alkyl or alkoxyl substituted with one or more halogen atoms.

As used herein, the term “optionally substituted haloalkyl” refers to unsubstituted haloalkyl having designated substituents replacing one or more hydrogen atoms on one or more hydrocarbon backbone carbon atoms. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

As used herein, the term “alkoxy” or “alkoxyl” includes substituted and unsubstituted alkyl, alkenyl and alkynyl groups covalently linked to an oxygen atom. Examples of alkoxy groups or alkoxyl radicals include, but are not limited to, methoxy, ethoxy, isopropyloxy, propoxy, butoxy and pentoxy groups. Examples of substituted alkoxy groups include halogenated alkoxy groups. The alkoxy groups can be substituted with groups such as alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moieties. Examples of halogen substituted alkoxy groups include, but are not limited to, fluoromethoxy, difluoromethoxy, trifluoromethoxy, chloromethoxy, dichloromethoxy and trichloromethoxy.

As used herein, the expressions “one or more of A, B, or C,” “one or more A, B, or C,” “one or more of A, B, and C,” “one or more A, B, and C,” “selected from the group consisting of A, B, and C”, “selected from A, B, and C”, and the like are used interchangeably and all refer to a selection from a group consisting of A, B, and/or C, i.e., one or more As, one or more Bs, one or more Cs, or any combination thereof, unless indicated otherwise.

It is to be understood that the present disclosure provides methods for the synthesis of the compounds of any of the Formulae described herein. The present disclosure also provides detailed methods for the synthesis of various disclosed compounds of the present disclosure according to the following schemes as well as those shown in the Examples.

It is to be understood that, throughout the description, where compositions are described as having, including, or comprising specific components, it is contemplated that compositions also consist essentially of, or consist of, the recited components. Similarly, where methods or processes are described as having, including, or comprising specific process steps, the processes also consist essentially of, or consist of, the recited processing steps. Further, it should be understood that the order of steps order for performing certain actions is immaterial so long as the invention remains operable. Moreover, two or more steps or actions can be conducted simultaneously.

It is to be understood that the synthetic processes of the disclosure can tolerate a wide variety of functional groups, therefore various substituted starting materials can be used. The processes generally provide the desired final compound at or near the end of the overall process, although it may be desirable in certain instances to further convert the compound to a pharmaceutically acceptable salt thereof.

It is to be understood that compounds of the present disclosure can be prepared in a variety of ways using commercially available starting materials, compounds known in the literature, or from readily prepared intermediates, by employing standard synthetic methods and procedures either known to those skilled in the art, or which will be apparent to the skilled artisan in light of the teachings herein. Standard synthetic methods and procedures for the preparation of organic molecules and functional group transformations and manipulations can be obtained from the relevant scientific literature or from standard textbooks in the field. Although not limited to any one or several sources, classic texts such as Smith, M. B., March, J., March's Advanced Organic Chemistry: Reactions, Mechanisms, and Structure, 5^{th} edition, John Wiley & Sons: New York, 2001; Greene, T. W., Wuts, P. G. M., Protective Groups in Organic Synthesis, 3^{rd} edition, John Wiley & Sons: New York, 1999; R. Larock, Comprehensive Organic Transformations, VCH Publishers (1989); L. Fieser and M. Fieser, Fieser andFieser's Reagents for Organic Synthesis, John Wiley and Sons (1994); and L. Paquette, ed., Encyclopedia of Reagents for Organic Synthesis, John Wiley and Sons (1995), incorporated by reference herein, are useful and recognised reference textbooks of organic synthesis known to those in the art.

One of ordinary skill in the art will note that, during the reaction sequences and synthetic schemes described herein, the order of certain steps may be changed, such as the introduction and removal of protecting groups. One of ordinary skill in the art will recognise that certain groups may require protection from the reaction conditions via the use of protecting groups. Protecting groups may also be used to differentiate similar functional groups in molecules. A list of protecting groups and how to introduce and remove these groups can be found in Greene, T. W., Wuts, P. G. M., Protective Groups in Organic Synthesis, 3^{rd} edition, John Wiley & Sons: New York, 1999.

It is to be understood that, unless otherwise stated, any description of a method of treatment or prevention includes use of the compounds to provide such treatment or prevention as is described herein. It is to be further understood, unless otherwise stated, any description of a method of treatment or prevention includes use of the compounds to prepare a medicament to treat or prevent such condition. The treatment or prevention includes treatment or prevention of human or non-human animals including rodents and other disease models.

### Compounds

Disclosed herein are lysergic acid diethylamide analogs, including 2-bromolysergic acid diethylamide, lysergic acid diethylamide, 1-Acetyl-N,N-diethyllysergamide, and 1-propionyl lysergic acid diethylamide, isotopically enriched at least one position.

2-Bromolysergic acid diethylamide has the formula

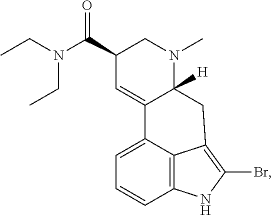 [SMILES: CCN(CC)C(=O)[C@@H]1C=C2c3cccc4[nH]c(Br)c(c34)C[C@H]2N(C)C1]

and also may be referred to as 2-Br-LSD.

Lysergic acid diethylamide has the formula

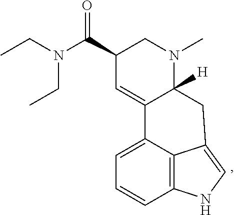 [SMILES: CCN(CC)C(=O)[C@@H]1C=C2c3cccc4[nH]cc(c34)C[C@H]2N(C)C1]

and also may be referred to as LSD.

1-Acetyl-N,N-diethyllysergamide has the structure

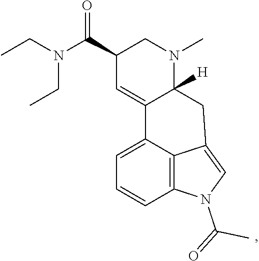 [SMILES: CCN(CC)C(=O)[C@@H]1C=C2c3cccc4c3c(cn4C(C)=O)C[C@H]2N(C)C1]

and also may be referred to as 1-acetyl-LSD, ALD-52 or 1A-LSD.

1-propionyl lysergic acid diethylamide has the structure

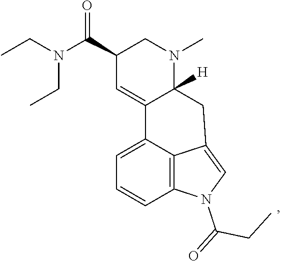 [SMILES: CCC(=O)n1cc2c3c(cccc31)C1=C[C@@H](C(=O)N(CC)CC)CN(C)[C@@H]1C2]

and also may be referred to as 1P-LSD.

Some embodiments of the isotopically enriched compounds disclosed herein have the formula,

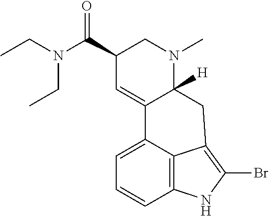 [SMILES: CCN(CC)C(=O)[C@@H]1C=C2c3cccc4[nH]c(Br)c(c34)C[C@H]2N(C)C1]

wherein at least an atom at one or more position in the formula is enriched in a stable isotope. In some embodiments, such compounds have improved properties relative to 2-Br-LSD, which does not have the drug-like pharmacokinetic and pharmacodynamic properties to support its use in the clinical treatment of brain disorders.

Some embodiments of the isotopically enriched compounds disclosed herein have the formula,

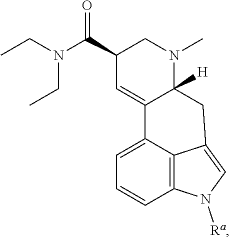

wherein R^{a} is selected from the group consisting of hydrogen, deuterium, tritium, acetyl and propionyl; and at least an atom at one or more position in the formula is enriched in a stable isotope, such as a heavy stable isotope, by way of example including those selected from ²H, ³H and ¹⁴C. In some embodiments, such isotopically enriched compounds have improved properties relative to LSD, ALD-52 and/or 1P-LSD, which do not have the drug-like pharmacokinetic and pharmacodynamic properties to support its use in the clinical treatment of brain disorders.

The present inventors observed that the metabolic properties of 2-Br-LSD, LSD, ALD-52, and 1P-LSD could be improved by isotopic enrichment, in particular, deuterium or tritium enrichment. In one embodiment of this approach, one attempts to slow the CYP-mediated metabolism of a drug or to reduce the formation of undesirable metabolites by replacing one or more protium (¹H) atoms with deuterium atoms. Deuterium is a safe, stable, non-radioactive isotope of hydrogen. Compared to protium, deuterium forms stronger bonds with carbon. In select cases, the increased bond strength imparted by deuterium can positively affect the pharmacokinetic properties of a drug, creating the potential for improved drug efficacy, safety, and/or tolerability. At the same time, because the size and shape of deuterium are essentially identical to those of protium, replacement of protium by deuterium would not be expected to affect the biochemical potency and selectivity of the drug as compared to the original chemical entity that contains only hydrogen. Tritium, ³H, forms still stronger bonds with carbon than deuterium. Thus, replacement of protium with tritium also can affect the pharmacokinetic properties of a molecule. Moreover, tritium is a beta emitter, meaning that enriching a molecule with tritium allows determination of pharmacokinetic and pharmacodynamic properties of the molecule to better understand its activity and ADME properties.

Accordingly, in some embodiments, the present invention provides a compound of Formula (A)

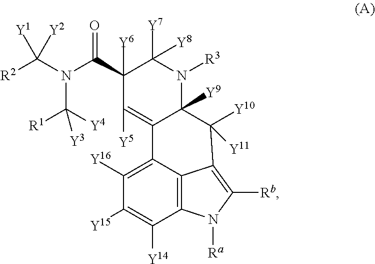

or a pharmaceutically acceptable salt thereof, wherein:
R¹, R², and R³ are methyl;
R^{a} is hydrogen, deuterium, tritium, acetyl, or 1-propionyl;
R^{b} is hydrogen, deuterium, tritium, or bromo;
Y¹, Y², Y³, Y⁴, Y⁵, Y⁶, Y⁷, Y⁸, Y⁹, Y¹⁰, Y¹¹, Y¹⁴, Y¹⁵, and Y¹⁶ are independently selected from hydrogen, deuterium, and tritium; and
at least one of R¹, R², R³, R^{a}, R^{b}, Y¹, Y², Y³, Y⁴, Y⁵, Y⁶, Y⁷, Y⁸, Y⁹, Y¹⁰, Y¹¹, Y¹⁴, Y¹⁵, and Y¹⁶ is or comprises deuterium or tritium.

In some embodiments, at least one of R¹, R², R³, R^{a}, R^{b}, Y¹, Y², Y³, Y⁴, Y⁵, Y⁶, Y⁷, Y⁸, Y⁹, Y¹⁰, Y¹¹, Y¹⁴, Y¹⁵, and Y¹⁶ is or comprises deuterium.

In one embodiment of Formula (A), R¹ is selected from CD₃, CD₂H, CDH₂, CT₃, CT₂H, CTH₂ and CH₃. In one embodiment of Formula (A), R¹, R² and R³ are selected from CD₃, CD₂H, CDH₂, CT₃, CT₂H, CTH₂ and CH₃. In one embodiment of 2-Br-LSD, LSD, ALD-52, and 1P-LSD analogs disclosed herein, such as 2-Br-LSD, LSD, ALD-52, and 1P-LSD analogs of Formula (A), Y¹, Y², Y³, Y⁴, Y⁵, Y⁶, Y⁷, Y⁸, Y⁹, Y¹⁰, Y¹¹, R^{b}, R^{a}, Y¹⁴ and Y¹⁵ are independently selected from protium, deuterium and tritium. In one embodiment of 2-Br-LSD, LSD, ALD-52, and 1P-LSD analogs disclosed herein, such as 2-Br-LSD, LSD, ALD-52, and 1P-LSD analogs of Formula (A), at least one of R¹, R², and R³ is enriched in deuterium. In some embodiments, at least one of Y¹, Y², Y³, Y⁴, Y⁵, Y⁶, Y⁷, Y⁸, Y⁹, Y¹⁰, Y¹¹, R^{b}, R^{a}, Y¹⁴ and Y¹⁵ is enriched in deuterium.

In some embodiments of Formula (A),
R^{b} is bromo;
Y¹⁴, Y¹⁵, and Y¹⁶ are deuterium;
R¹, R², and R³ are independently selected from CD₃, CD₂H, CDH₂, and CH₃; and
R^{a}, Y¹, Y², Y³, Y⁴, Y⁵, Y⁶, Y⁷, Y⁸, Y⁹, Y¹⁰, and Y¹¹ are independently hydrogen or deuterium.

In some embodiments of Formula (A),
R^{b} is bromo;
R³ is CD₃;
R¹ and R² are independently selected from CD₃, CD₂H, CDH₂, and CH₃; and
R^{a}, Y¹, Y², Y³, Y⁴, Y⁵, Y⁶, Y⁷, Y⁸, Y⁹, Y¹⁰, Y¹¹, Y¹⁴, Y¹⁵, and Y¹⁶ are independently hydrogen or deuterium.

In some embodiments of Formula (A),
R^{b} is bromo;
R¹ and R² are CD₃;
Y¹, Y², Y³, and Y⁴ are deuterium;
R³ is selected from CD₃, CD₂H, CDH₂, and CH₃; and
R^{a}, Y⁵, Y⁶, Y⁷, Y⁸, Y⁹, Y¹⁰, Y¹¹, Y¹⁴, Y¹⁵, and Y¹⁶ are independently hydrogen or deuterium.

In some embodiments of Formula (A),
R^{b} is bromo;
R¹, R², and R³ are CD₃;
Y¹, Y², Y³, and Y⁴ are deuterium; and
R^{a}, Y⁵, Y⁶, Y⁷, Y⁸, Y⁹, Y¹⁰, Y¹¹, Y¹⁴, Y¹⁵, and Y¹⁶ are independently hydrogen or deuterium.

In some embodiments of Formula (A),
R¹ and R² are CD₃;
Y¹, Y², Y³, and Y⁴ are deuterium;
R³ is selected from CD₃, CD₂H, CDH₂, and CH₃; and
R^{a}, R^{b}, Y⁵, Y⁶, Y⁷, Y⁸, Y⁹, Y¹⁰, Y¹¹, Y¹⁴, Y¹⁵, and Y¹⁶ are independently hydrogen or deuterium.

In some embodiments of Formula (A),
R¹ and R² are CD₃;
R^{b}, Y¹, Y², Y³, Y⁴, Y¹⁴, Y¹⁵, and Y¹⁶ are deuterium;
R³ is selected from CD₃, CD₂H, CDH₂, and CH₃; and
R^{a}, Y⁵, Y⁶, Y⁷, Y⁸, Y⁹, Y¹⁰, and Y¹¹ are independently hydrogen or deuterium.

In some embodiments of Formula (A),
R¹ and R² are CD₃;
R^{b}, Y¹, Y², Y³, and Y⁴ are deuterium;
R³ is selected from CD₃, CD₂H, CDH₂, and CH₃; and
R^{a}, Y⁵, Y⁶, Y⁷, Y⁸, Y⁹, Y¹⁰, Y¹¹, Y¹⁴, Y¹⁵, and Y¹⁶ are independently hydrogen or deuterium.

In some embodiments of Formula (A),
R¹, R², and R³ are CD₃;
Y¹, Y², Y³, and Y⁴ are deuterium; and
R^{a}, R^{b}, Y⁵, Y⁶, Y⁷, Y⁸, Y⁹, Y¹⁰, Y¹¹, Y¹⁴, Y¹⁵, and Y¹⁶ are independently hydrogen or deuterium.

In some embodiments of Formula (A),
R^{b}, Y¹⁴, Y¹⁵, and Y¹⁶ are deuterium;
R¹, R², and R³ are independently selected from CD₃, CD₂H, CDH₂, and CH₃; and
R^{a}, Y¹, Y², Y³, Y⁴, Y⁵, Y⁶, Y⁷, Y⁸, Y⁹, Y¹⁰, and Y¹¹ are independently hydrogen or deuterium.

In some embodiments of Formula (A),
R^{b} is deuterium;
R¹, R², and R³ are independently selected from CD₃, CD₂H, CDH₂, and CH₃; and
R^{a}, Y¹, Y², Y³, Y⁴, Y⁵, Y⁶, Y⁷, Y⁸, Y⁹, Y¹⁰, Y¹¹, Y¹⁴, Y¹⁵, and Y¹⁶ are independently hydrogen or deuterium.

In some embodiments of Formula (A),
R³ is CD₃;
R¹ and R² are independently selected from CD₃, CD₂H, CDH₂, and CH₃; and
R^{a}, R^{b}, Y¹, Y², Y³, Y⁴, Y⁵, Y⁶, Y⁷, Y⁸, Y⁹, Y¹⁰, Y¹¹, Y¹⁴, Y¹⁵, and Y¹⁶ are independently hydrogen or deuterium.

In some embodiments, the present invention provides an isotopically enriched compound of the formula:

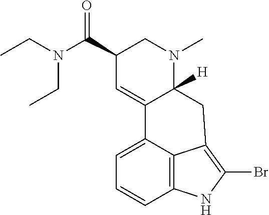 [SMILES: CCN(CC)C(=O)[C@@H]1C=C2c3cccc4[nH]c(Br)c(c34)C[C@H]2N(C)C1]

wherein at least one position is enriched a heavy isotope, such as wherein the heavy isotope is selected from ¹⁴C, tritium and deuterium.

Compounds according to some embodiments disclosed herein are represented by Formula (I)

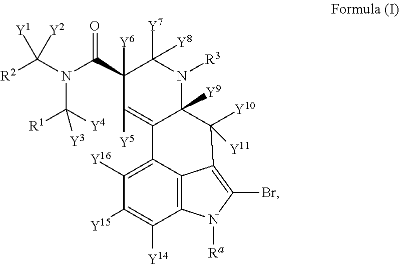

wherein R¹, R² and R³ are methyl; Y¹, Y², Y³, Y⁴, Y⁵, Y⁶, Y⁷, Y⁸, Y⁹, Y¹⁰, Y¹¹, R^{a}, Y¹⁴, Y¹⁵ and Y¹⁶ each are hydrogen, deuterium or tritium; and at least one of R¹, R², R³, Y¹, Y², Y³, Y⁴, Y⁵, Y⁶, Y⁷, Y⁸, Y⁹, Y¹⁰, Y¹¹, R^{a}, Y¹⁴, Y¹⁵ and Y¹⁶ is enriched in at least one heavy isotope, such as wherein the heavy isotope is deuterium.

In some embodiments, at least one of Y¹, Y², Y³, Y⁴, Y⁵, Y⁶, Y⁷, Y⁸, Y⁹, Y¹⁰, Y¹¹, R^{a}, Y¹⁴, Y¹⁵ and Y¹⁶ of Formula (I) is deuterium.

In one embodiment of Formula I, R¹ is selected from CD₃, CD₂H, CDH₂, CT₃, CT₂H, CTH₂ and CH₃. In one embodiment of Formula I, R¹, R² and R³ are selected from CD₃, CD₂H, CDH₂, CT₃, CT₂H, CTH₂ and CH₃. In one embodiment of 2-Br-LSD analogs disclosed herein, such as 2-Br-LSD analogs of Formula (I), Y¹, Y², Y³, Y⁴, Y⁵, Y⁶, Y⁷, Y⁸, Y⁹, Y¹⁰, Y¹¹, R^{a}, Y¹⁴, Y¹⁵ and Y¹⁶ are independently selected from protium, deuterium and tritium. In one embodiment of 2-Br-LSD analogs disclosed herein, such as 2-Br-LSD analogs of Formula (I), at least one of R¹, R², and R³ is enriched in deuterium. In some embodiments, at least one of Y, Y², Y³, Y⁴, Y⁵, Y⁶, Y⁷, Y⁸, Y⁹, Y¹⁰, Y¹¹, R^{a}, Y¹⁴, Y¹⁵ and Y¹⁶ is enriched in deuterium.

In some embodiments, the present invention provides an isotopically enriched compound of Formula (II)

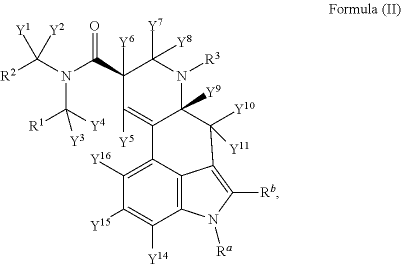

wherein
R¹, R² and R³ are methyl; Y¹, Y², Y³, Y⁴, Y⁵, Y⁶, Y⁷, Y⁸, Y⁹, Y¹⁰, Y¹¹, R^{b}, Y¹⁴, Y¹⁵ and Y¹⁶ each are hydrogen, deuterium, or tritium; and R^{a} is selected from the group consisting of hydrogen, deuterium, tritium, acetyl and 1-propionyl. In one embodiment, an atom at least one position is enriched in a heavy isotope, such as a heavy isotope selected from ¹⁴C, tritium and deuterium.

In some embodiments, at least one of Y¹, Y², Y³, Y⁴, Y⁵, Y⁶, Y⁷, Y⁸, Y⁹, Y¹⁰, Y¹¹, R^{b}, Y¹⁴, Y¹⁵ and Y¹⁶ of Formula (II) is deuterium.

In one embodiment of Formula (II) disclosed herein, at least one of R¹, R², R³, Y¹, Y², Y³, Y⁴, Y⁵, Y⁶, Y⁷, Y⁸, Y⁹, Y¹⁰, Y¹¹, R^{b}, Y¹⁴, Y¹⁵, Y¹⁶ and R^{a} is enriched in at least one heavy isotope.

In one embodiment of compounds according to Formula (II), compounds disclosed herein have Formula (IIa)

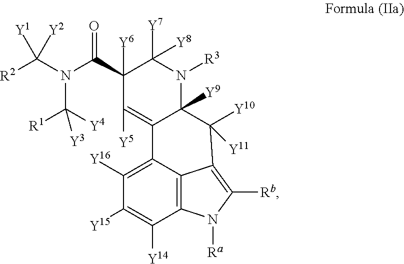

wherein R¹, R² and R³ are methyl; and Y¹, Y², Y³, Y⁴, Y⁵, Y⁶, Y⁷, Y⁸, Y⁹, Y¹⁰, Y¹¹, R^{b},
R^{a}, Y¹⁴, Y¹⁵ and Y¹⁶ each are hydrogen, deuterium, or tritium. In one embodiment of Formulas (II) and (IIa), at least one of Y¹, Y², Y³, Y⁴, Y⁵, Y⁶, Y⁷, Y⁸, Y⁹, Y¹⁰, Y¹¹, R^{b}, R^{a}, Y¹⁴, Y¹⁵ and Y¹⁶ is enriched in deuterium. In one embodiment of Formulas (II) and (IIa), at least one of R¹, R² and R³ are enriched in deuterium. In one embodiment of Formulas (II) and (IIa), at least one of R¹, R², R³, Y¹, Y², Y³, Y⁴, Y⁵, Y⁶, Y⁷, Y⁸, Y⁹, Y¹⁰, Y¹¹, R^{b}, R^{a}, Y¹⁴, Y¹⁵ and Y¹⁶ is enriched in deuterium.

In some embodiments, at least one of Y¹, Y², Y³, Y⁴, Y⁵, Y⁶, Y⁷, Y⁸, Y⁹, Y¹⁰, Y, R^{b}, R^{a}, Y¹⁴, Y¹⁵ and Y¹⁶ of Formula (IIa) is deuterium.

In a particular embodiment of Formulas (II) and (IIa), R¹, R² and R³ are selected from CD₃, CD₂H, CDH₂, CT₃, CT₂H, CTH₂ and CH₃. In one embodiment of Formulas (II) and (IIa), Y¹, Y², Y³, Y⁴, Y⁵, Y⁶, Y⁷, Y⁸, Y⁹, Y¹⁰, Y¹¹, R^{b}, R^{a}, Y¹⁴, Y¹⁵ and Y¹⁶ are selected from protium, deuterium and tritium.

In another embodiment of compounds according to Formula (II), compounds disclosed herein have Formula (IIb)

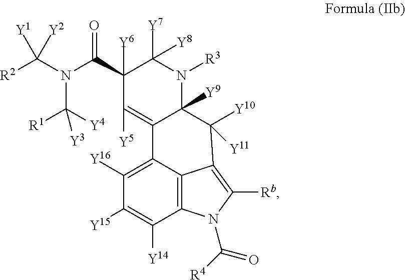

wherein R¹, R² and R³; are methyl, R⁴ is methyl or ethyl; and Y¹, Y², Y³, Y⁴, Y⁵, Y⁶, Y⁷, Y⁸, Y⁹, Y¹⁰, Y¹¹, R^{b}, Y¹⁴, Y¹⁵ and Y¹⁶ each are hydrogen, deuterium, or tritium.

In some embodiments, at least one of Y¹, Y², Y³, Y⁴, Y⁵, Y⁶, Y⁷, Y⁸, Y⁹, Y¹⁰, Y¹¹, R^{b}, Y¹⁴, Y¹⁵ and Y¹⁶ of Formula (IIb) is deuterium.

In one embodiment of Formulas (II) and (IIb), compounds disclosed herein have Formula (IIc)

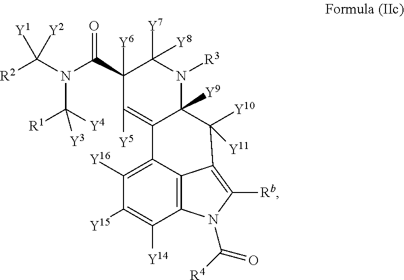

wherein R¹, R², R³ are methyl; R⁴ is ethyl; and Y¹, Y², Y³, Y⁴, Y⁵, Y⁶, Y⁷, Y⁸, Y⁹, Y¹⁰, Y¹¹, R^{b}, Y¹⁴, Y¹⁵ and Y¹⁶ each are hydrogen, deuterium, or tritium.

In some embodiments, at least one of Y¹, Y², Y³, Y⁴, Y⁵, Y⁶, Y⁷, Y, Y⁹, Y¹⁰, Y¹¹, R^{b}, Y¹⁴, Y¹⁵ and Y¹⁶ of Formula (IIc) is deuterium.

In one embodiment of LSD, ALD-52 and 1P-LSD analogs disclosed herein, such as LSD and ALD-52 analogs of Formula (II), at least one of R¹, R², and R³ is enriched in deuterium. In one embodiment of LSD, ALD-52 and 1P-LSD analogs disclosed herein, including compounds of Formula (II), at least one of R¹, R², R³, and R^{a} are enriched in deuterium, such as wherein R^{a} is an acetyl enriched in deuterium.

In some embodiments of Formula (IIa), at least one of Y, Y², Y³, Y⁴, Y⁵, Y⁶, Y⁷, Y⁵, Y⁹, Y¹⁰, Y¹¹, R^{b}, R^{a}, Y¹⁴, Y¹⁵ and Y¹⁶ is enriched in deuterium. In one embodiment of Formulas (II), (IIa) and (IIb), at least one of R¹, R² and R³ is enriched in deuterium. In a particular embodiment of Formulas (II), (IIa) and (IIb), R¹, R² and R³ are selected from CD₃, CD₂H, CDH₂, CT₃, CT₂H and CTH₂. In one embodiment of Formulas (II), (IIa) and (IIb), at least one of Y¹, Y², Y³, Y⁴, is enriched in deuterium. In one embodiment of Formulas (II), (IIa) and (IIb), at least one of Y¹, Y², Y³, Y⁴, is enriched in deuterium and at least one of R¹, R², R³, and R^{a} is enriched in deuterium.

In some embodiments of Formulas (II), (IIb) and (Ic), at least one of Y, Y², Y³, Y⁴, Y⁵, Y⁶, Y⁷, Y⁸, Y⁹, Y¹⁰, Y¹¹, R^{b}, Y¹⁴ and Y¹⁵ is enriched in deuterium. In some embodiments of Formulas (I), (Ib) and (Ic), at least one of at least one of Y¹, Y², Y³, Y⁴, is enriched in deuterium. In some embodiments of Formulas (II), (IIb) and (IIc), R¹, R² and R³ are selected from CD₃, CD₂H, CDH₂, CT₃, CT₂H and CTH₂. In some embodiments, R⁴ is CD₂CD₃, CHDCD₃, CH₂CD₃, CH₂CD₃, CD₂CHD₂, CHDCHD₂, CH₂CHD₂, CD₂CH₂D, CHDCH₂D, CH₂CH₂D, CD₂CH₃, CHDCH₃, or CH₂CH₃. In some embodiments, R⁴ is CD₂CD₃ or CH₂CH₃. In some embodiments, R⁴ is CD₃, CD₂H, CDH₂, CT₃, CT₂H and CTH₂. In some embodiments, R⁴ is CD₂CD₃, CH₂CH₃, CD₃, or CH₃.

In some embodiments of 2-Br-LSD analogs disclosed herein, such as 2-Br-LSD analogs of Formula (I), disclosed compounds are selected from the group consisting of:

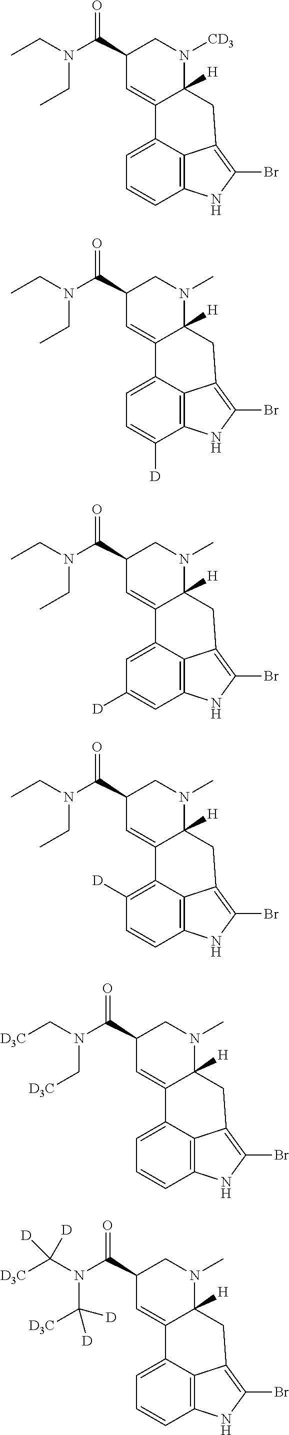

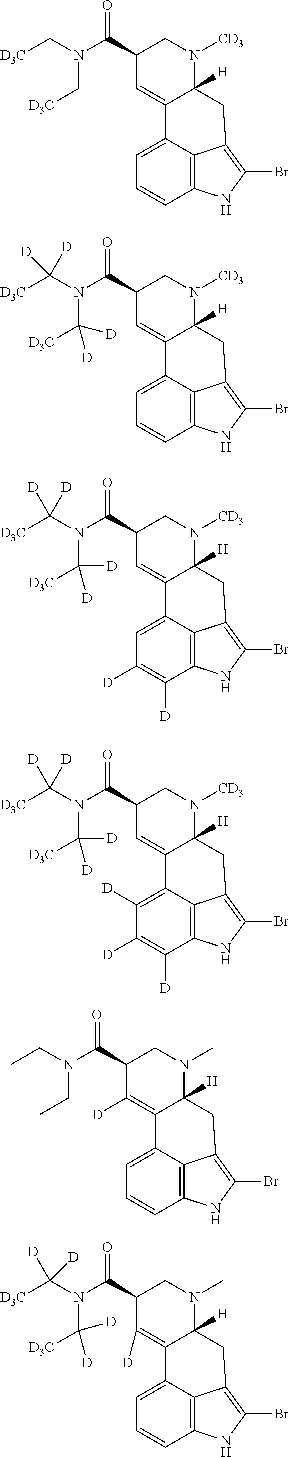

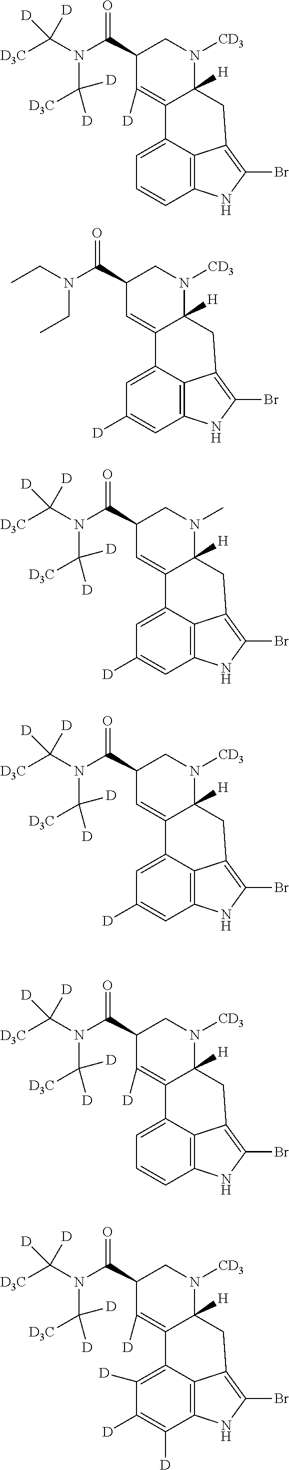

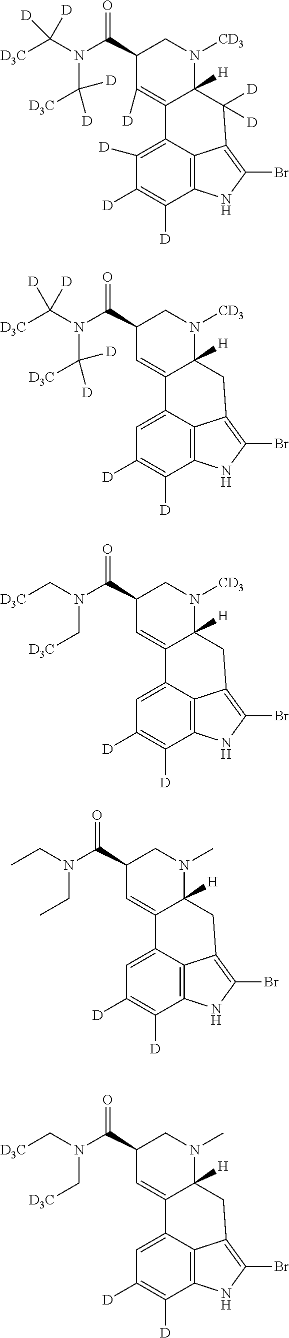

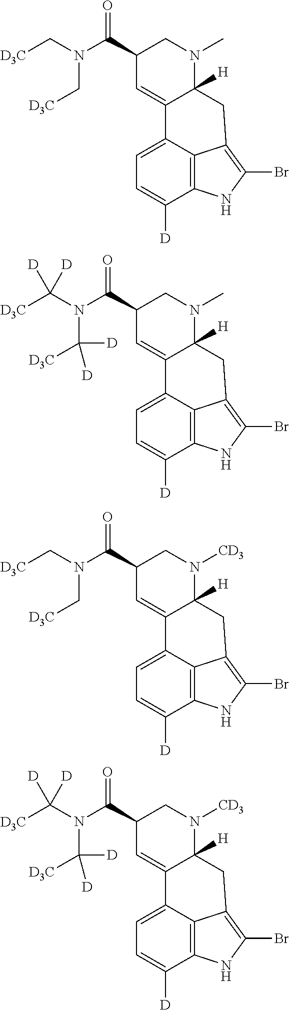 [SMILES: [2H]c1ccc2c3c(c(Br)[nH]c13)C[C@@H]1C2=C[C@@H](C(=O)N(CC([2H])([2H])[2H])CC([2H])([2H])[2H])CN1C ; [2H]c1ccc2c3c(c(Br)[nH]c13)C[C@@H]1C2=C[C@@H](C(=O)N(C([2H])([2H])C([2H])([2H])[2H])C([2H])([2H])C([2H])([2H])[2H])CN1C ; [2H]c1ccc2c3c(c(Br)[nH]c13)C[C@@H]1C2=C[C@@H](C(=O)N(CC([2H])([2H])[2H])CC([2H])([2H])[2H])CN1C([2H])([2H])[2H] ; [2H]c1ccc2c3c(c(Br)[nH]c13)C[C@@H]1C2=C[C@@H](C(=O)N(C([2H])([2H])C([2H])([2H])[2H])C([2H])([2H])C([2H])([2H])[2H])CN1C([2H])([2H])[2H]]

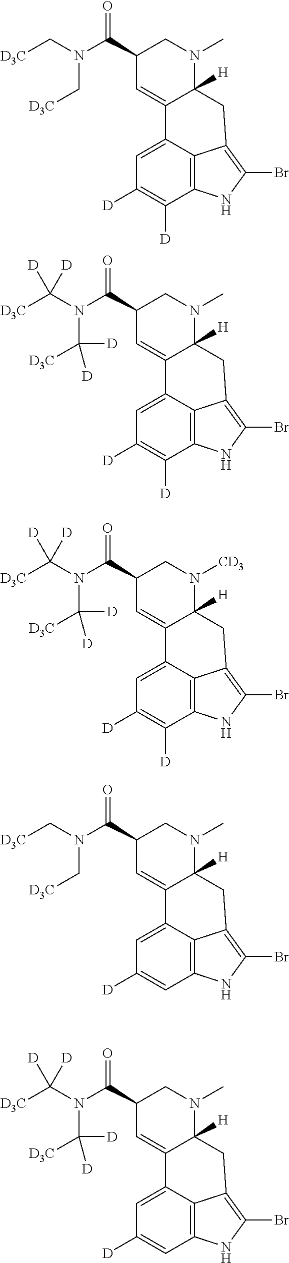

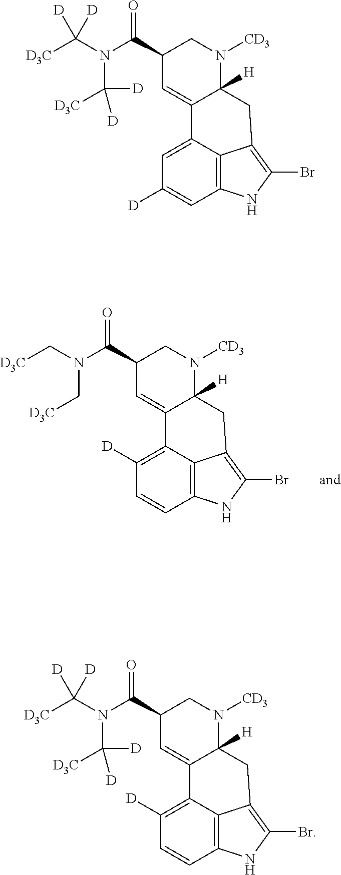 [SMILES: [2H]c1cc2c3c(c(Br)[nH]c3c1)C[C@@H]1C2=C[C@@H](C(=O)N(C([2H])([2H])C([2H])([2H])[2H])C([2H])([2H])C([2H])([2H])[2H])CN1C([2H])([2H])[2H] ; [2H]c1ccc2[nH]c(Br)c3c2c1C1=C[C@@H](C(=O)N(CC([2H])([2H])[2H])CC([2H])([2H])[2H])CN(C([2H])([2H])[2H])[C@@H]1C3 ; "and" ; [2H]c1ccc2[nH]c(Br)c3c2c1C1=C[C@@H](C(=O)N(C([2H])([2H])C([2H])([2H])[2H])C([2H])([2H])C([2H])([2H])[2H])CN(C([2H])([2H])[2H])[C@@H]1C3]

In some embodiments of LSD, ALD-52 and 1P-LSD analogs disclosed herein, such as LSD, ALD-52 and 1P-LSD analogs of Formula (II), disclosed compounds are selected from the group consisting of:

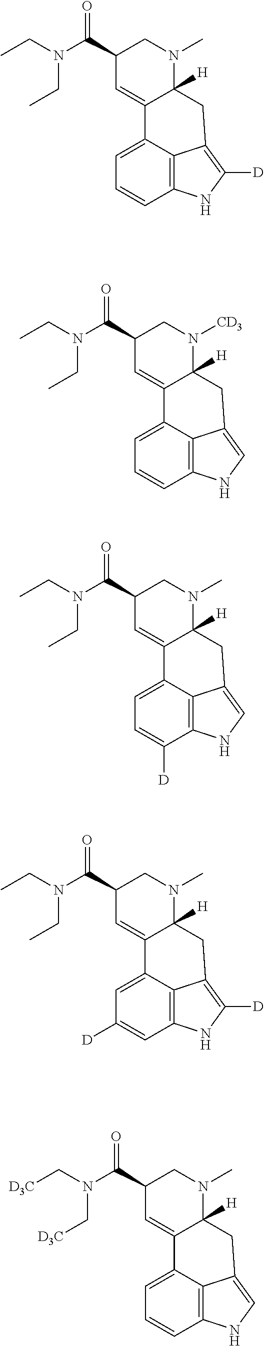 [SMILES: [2H]c1[nH]c2cccc3c2c1C[C@@H]1C3=C[C@@H](C(=O)N(CC)CC)CN1C ; [2H]C([2H])([2H])N1C[C@H](C(=O)N(CC)CC)C=C2c3cccc4[nH]cc(c34)C[C@H]21 ; [2H]c1ccc2c3c(c[nH]c13)C[C@@H]1C2=C[C@@H](C(=O)N(CC)CC)CN1C ; [2H]c1cc2c3c(c([2H])[nH]c3c1)C[C@@H]1C2=C[C@@H](C(=O)N(CC)CC)CN1C ; [2H]C([2H])([2H])CN(CC([2H])([2H])[2H])C(=O)[C@@H]1C=C2c3cccc4[nH]cc(c34)C[C@H]2N(C)C1]

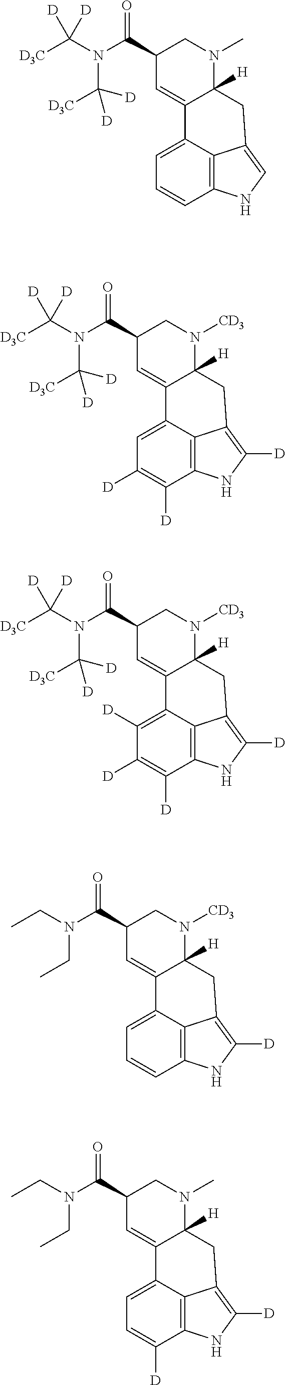

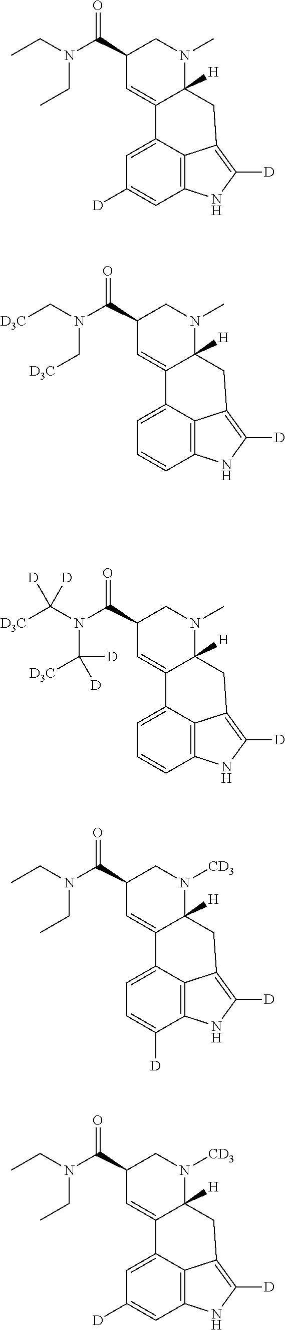 [SMILES: [2H]c1cc2c3c(c([2H])[nH]c3c1)C[C@@H]1C2=C[C@@H](C(=O)N(CC)CC)CN1C ; [2H]c1[nH]c2cccc3c2c1C[C@@H]1C3=C[C@@H](C(=O)N(CC([2H])([2H])[2H])CC([2H])([2H])[2H])CN1C ; [2H]c1[nH]c2cccc3c2c1C[C@@H]1C3=C[C@@H](C(=O)N(C([2H])([2H])C([2H])([2H])[2H])C([2H])([2H])C([2H])([2H])[2H])CN1C ; [2H]c1[nH]c2c([2H])ccc3c2c1C[C@@H]1C3=C[C@@H](C(=O)N(CC)CC)CN1C([2H])([2H])[2H] ; [2H]c1cc2c3c(c([2H])[nH]c3c1)C[C@@H]1C2=C[C@@H](C(=O)N(CC)CC)CN1C([2H])([2H])[2H]]

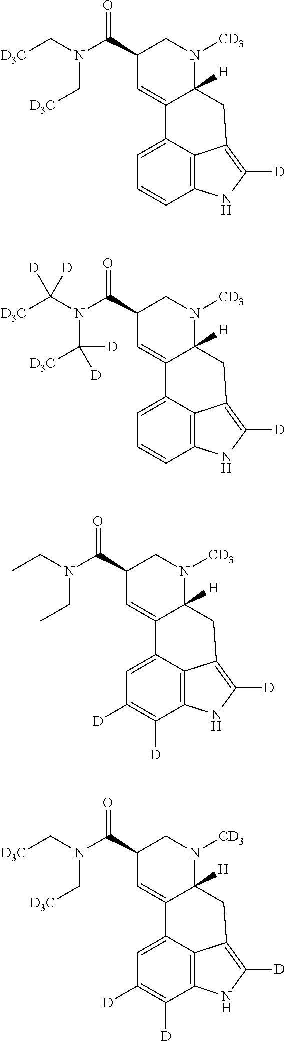 [SMILES: [2H]c1[nH]c2cccc3c2c1C[C@@H]1C3=C[C@@H](C(=O)N(CC([2H])([2H])[2H])CC([2H])([2H])[2H])CN1C([2H])([2H])[2H] ; [2H]c1[nH]c2cccc3c2c1C[C@@H]1C3=C[C@@H](C(=O)N(C([2H])([2H])C([2H])([2H])[2H])C([2H])([2H])C([2H])([2H])[2H])CN1C([2H])([2H])[2H] ; [2H]c1cc2c3c(c([2H])[nH]c3c1[2H])C[C@@H]1C2=C[C@@H](C(=O)N(CC)CC)CN1C([2H])([2H])[2H] ; [2H]c1cc2c3c(c([2H])[nH]c3c1[2H])C[C@@H]1C2=C[C@@H](C(=O)N(CC([2H])([2H])[2H])CC([2H])([2H])[2H])CN1C([2H])([2H])[2H]]

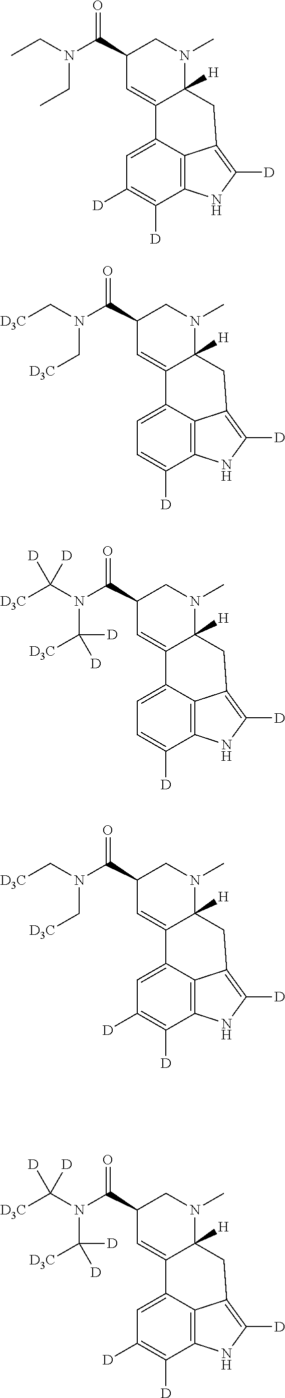

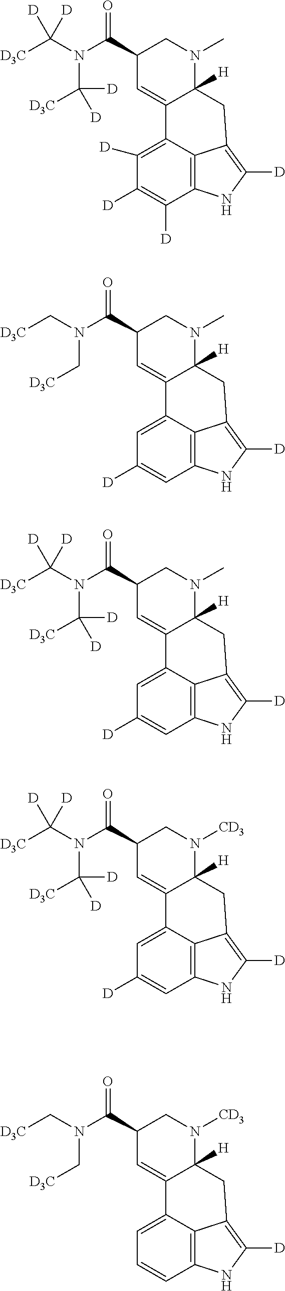

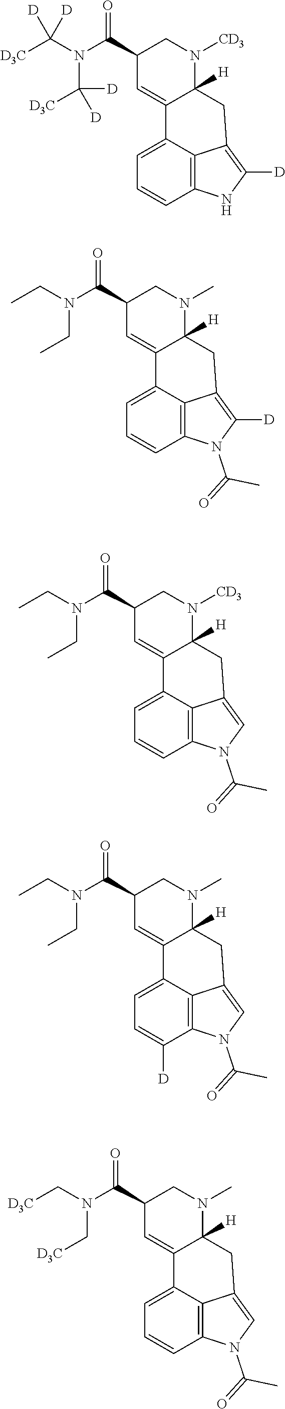

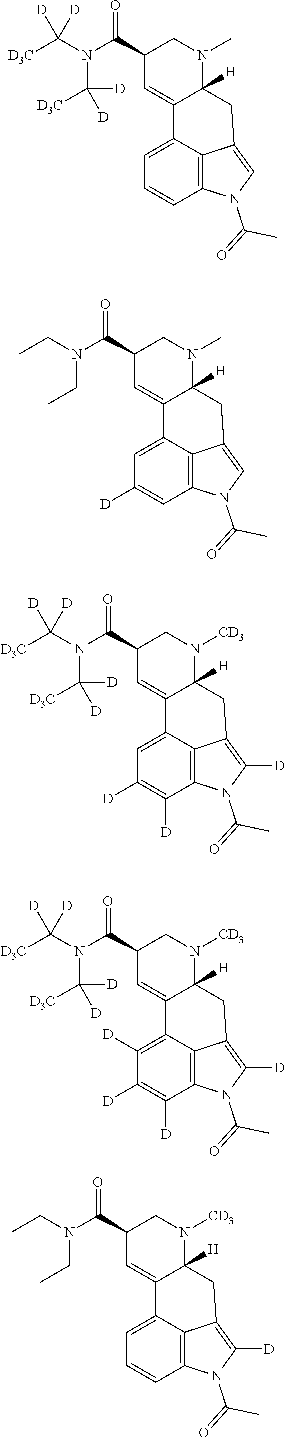

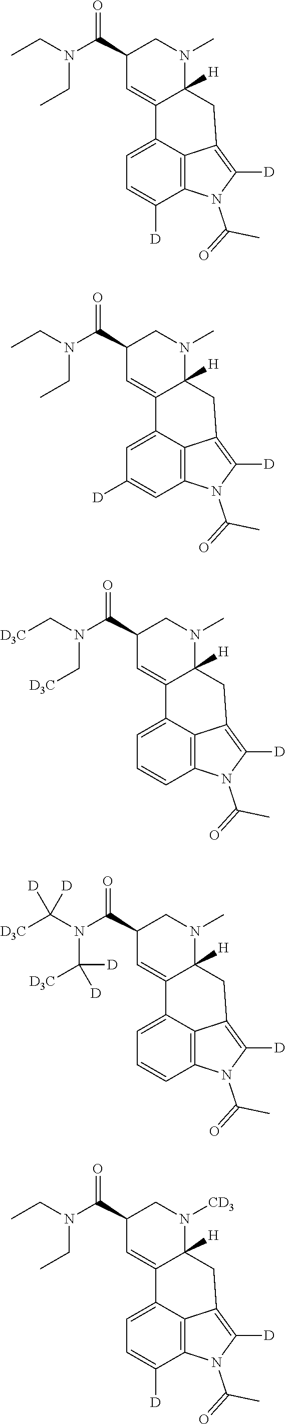 [SMILES: [2H]c1ccc2c3c(c([2H])n(C(C)=O)c13)C[C@@H]1C2=C[C@@H](C(=O)N(CC)CC)CN1C ; [2H]c1cc2c3c(c([2H])n(C(C)=O)c3c1)C[C@@H]1C2=C[C@@H](C(=O)N(CC)CC)CN1C ; [2H]c1c2c3c(cccc3n1C(C)=O)C1=C[C@@H](C(=O)N(CC([2H])([2H])[2H])CC([2H])([2H])[2H])CN(C)[C@@H]1C2 ; [2H]c1c2c3c(cccc3n1C(C)=O)C1=C[C@@H](C(=O)N(C([2H])([2H])C([2H])([2H])[2H])C([2H])([2H])C([2H])([2H])[2H])CN(C)[C@@H]1C2 ; [2H]c1ccc2c3c(c([2H])n(C(C)=O)c13)C[C@@H]1C2=C[C@@H](C(=O)N(CC)CC)CN1C([2H])([2H])[2H]]

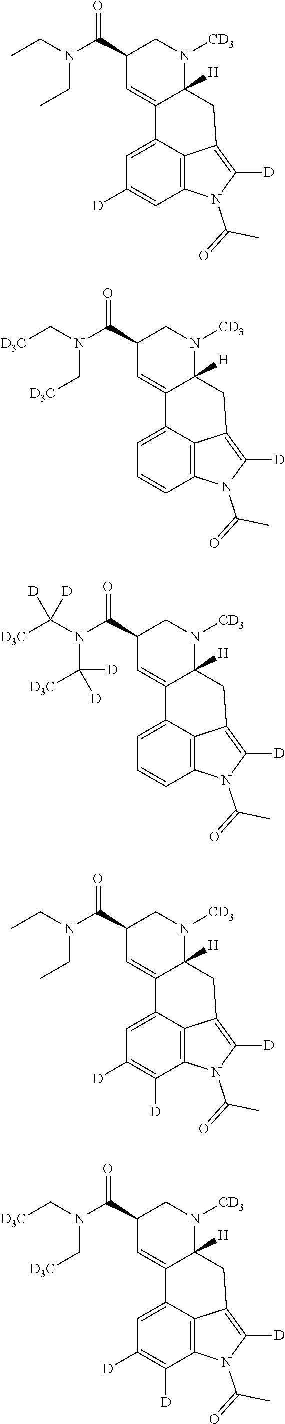

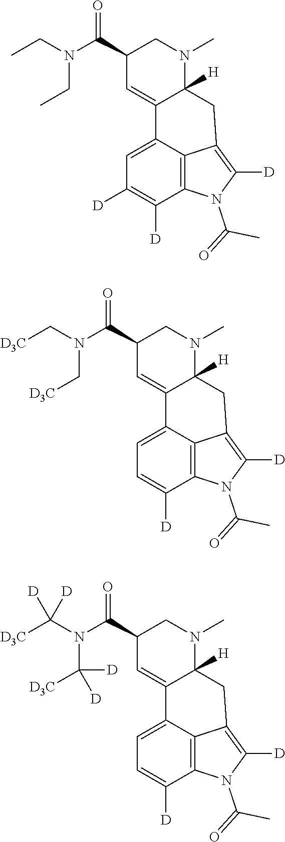 [SMILES: [2H]c1cc2c3c(c([2H])n(C(C)=O)c3c1[2H])C[C@@H]1C2=C[C@@H](C(=O)N(CC)CC)CN1C ; [2H]c1ccc2c3c(c([2H])n(C(C)=O)c13)C[C@@H]1C2=C[C@@H](C(=O)N(CC([2H])([2H])[2H])CC([2H])([2H])[2H])CN1C ; [2H]c1ccc2c3c(c([2H])n(C(C)=O)c13)C[C@@H]1C2=C[C@@H](C(=O)N(C([2H])([2H])C([2H])([2H])[2H])C([2H])([2H])C([2H])([2H])[2H])CN1C]

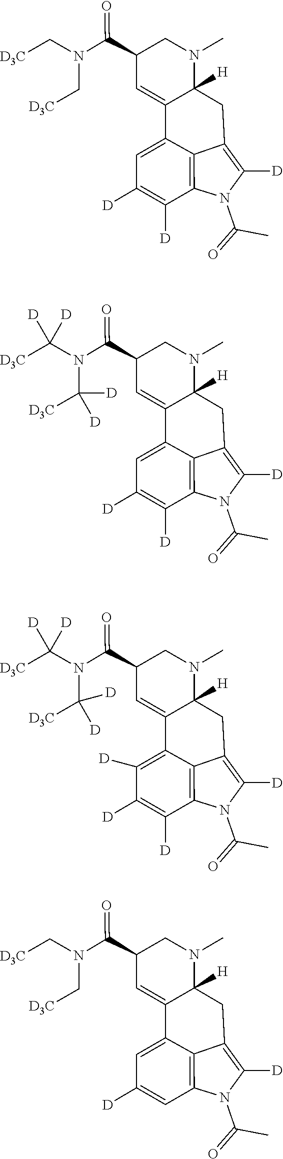 [SMILES: [2H]c1cc2c3c(c([2H])n(C(C)=O)c3c1[2H])C[C@@H]1C2=C[C@@H](C(=O)N(CC([2H])([2H])[2H])CC([2H])([2H])[2H])CN1C ; [2H]c1cc2c3c(c([2H])n(C(C)=O)c3c1[2H])C[C@@H]1C2=C[C@@H](C(=O)N(C([2H])([2H])C([2H])([2H])[2H])C([2H])([2H])C([2H])([2H])[2H])CN1C ; [2H]c1c([2H])c([2H])c2c3c(c([2H])n2C(C)=O)C[C@@H]2C(=C[C@@H](C(=O)N(C([2H])([2H])C([2H])([2H])[2H])C([2H])([2H])C([2H])([2H])[2H])CN2C)c13 ; [2H]c1cc2c3c(c([2H])n(C(C)=O)c3c1)C[C@@H]1C2=C[C@@H](C(=O)N(CC([2H])([2H])[2H])CC([2H])([2H])[2H])CN1C]

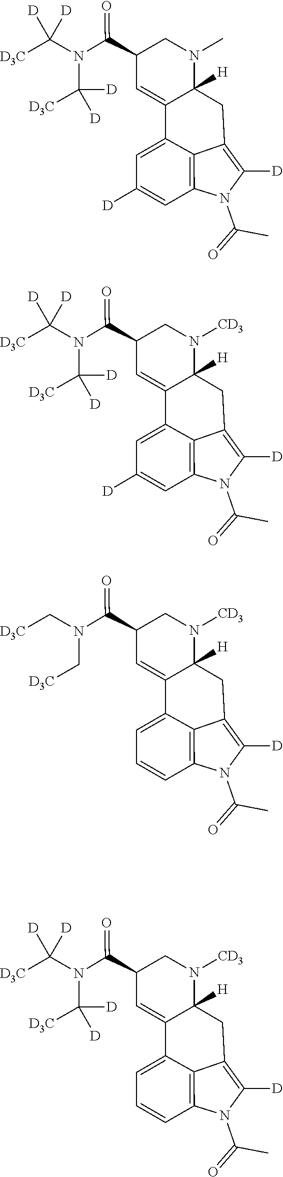

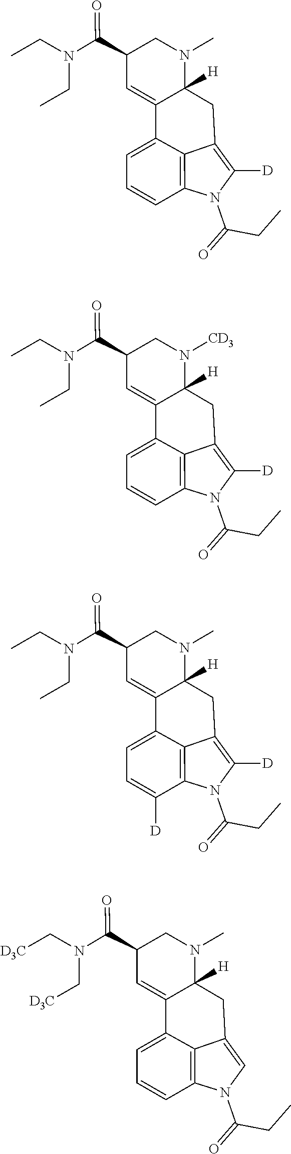

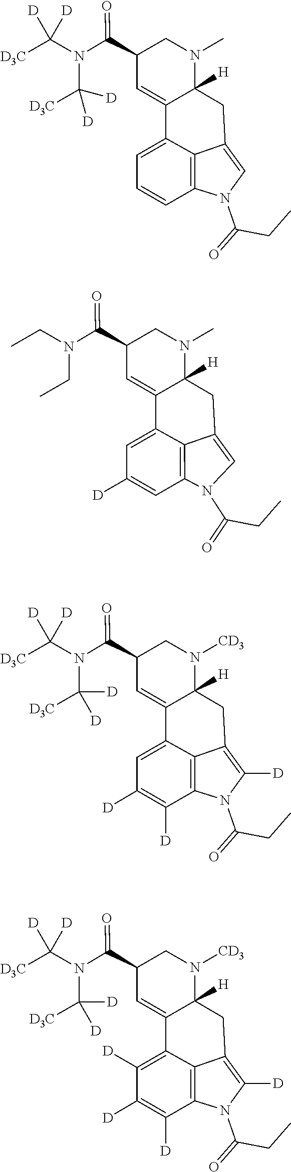

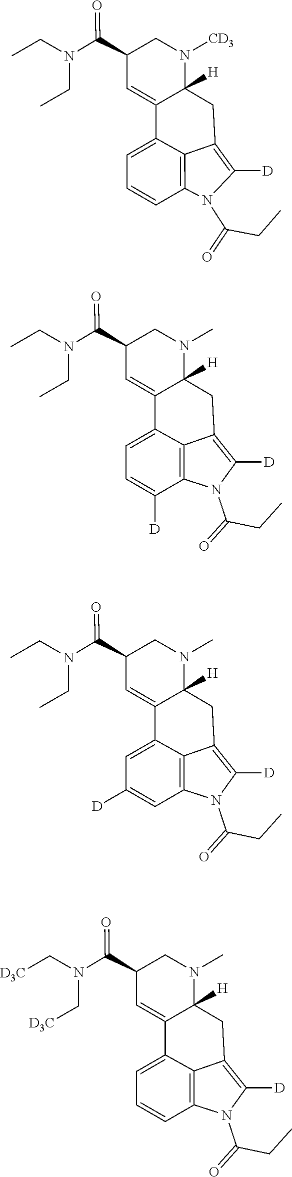

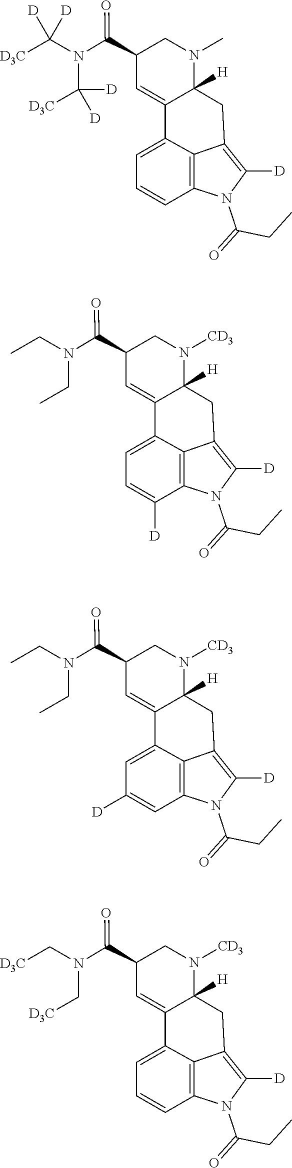

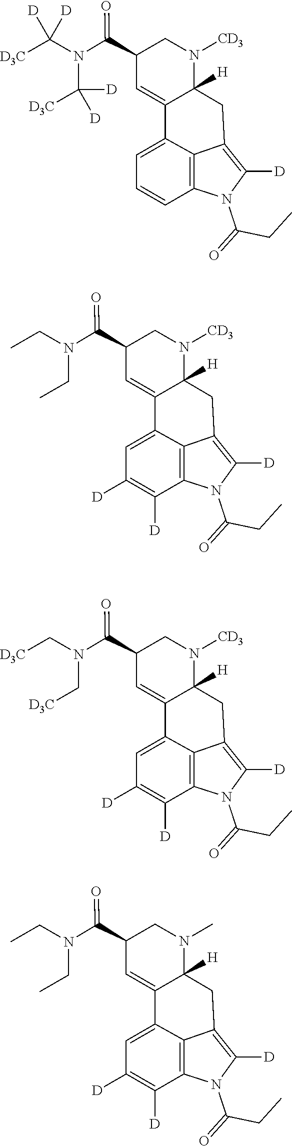

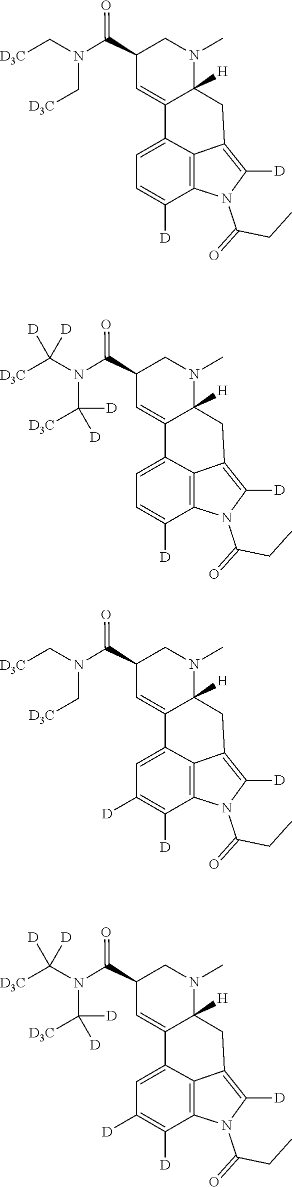 [SMILES: [2H]c1ccc2c3c(c([2H])n(C(=O)CC)c13)C[C@@H]1C2=C[C@@H](C(=O)N(CC([2H])([2H])[2H])CC([2H])([2H])[2H])CN1C ; [2H]c1ccc2c3c(c([2H])n(C(=O)CC)c13)C[C@@H]1C2=C[C@@H](C(=O)N(C([2H])([2H])C([2H])([2H])[2H])C([2H])([2H])C([2H])([2H])[2H])CN1C ; [2H]c1cc2c3c(c([2H])n(C(=O)CC)c3c1[2H])C[C@@H]1C2=C[C@@H](C(=O)N(CC([2H])([2H])[2H])CC([2H])([2H])[2H])CN1C ; [2H]c1cc2c3c(c([2H])n(C(=O)CC)c3c1[2H])C[C@@H]1C2=C[C@@H](C(=O)N(C([2H])([2H])C([2H])([2H])[2H])C([2H])([2H])C([2H])([2H])[2H])CN1C]

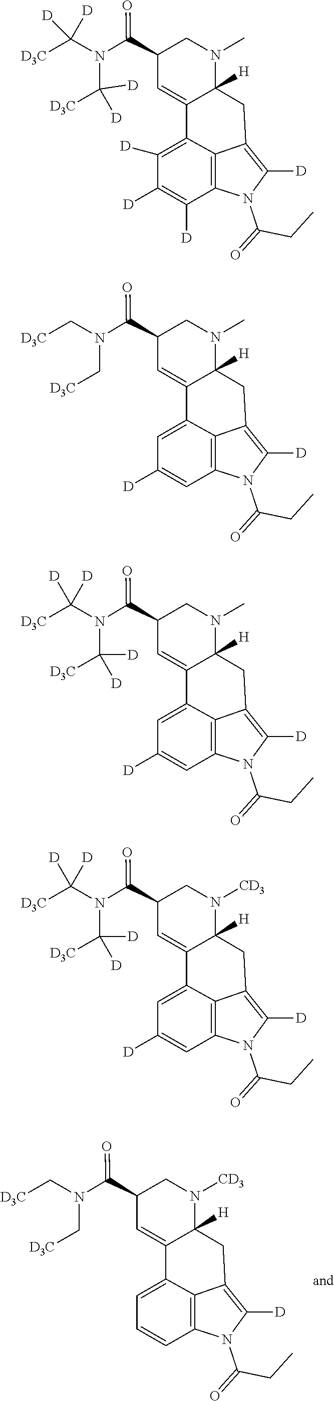

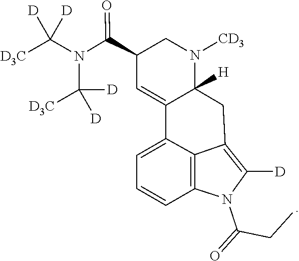 [SMILES: [2H]c1c2c3c(cccc3n1C(=O)CC)C1=C[C@@H](C(=O)N(C([2H])([2H])C([2H])([2H])[2H])C([2H])([2H])C([2H])([2H])[2H])CN(C([2H])([2H])[2H])[C@@H]1C2]

Representative deuterated versions of 2-Br-LSD, LSD, ALD-52 and 1P-LSD are included in Table 1.

TABLE 1

| Cpd | |
|---|---|
| Number | Structure |
| 1 | 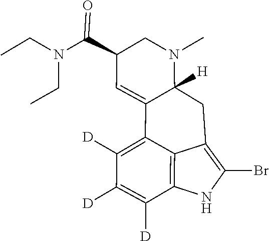 [SMILES: [2H]c1c([2H])c2c3c(c(Br)[nH]c3c1[2H])C[C@@H]1C2=C[C@@H](C(=O)N(CC)CC)CN1C] |
| 2 | 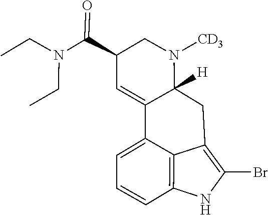 [SMILES: [2H]C([2H])([2H])N1C[C@H](C(=O)N(CC)CC)C=C2c3cccc4[nH]c(Br)c(c34)C[C@H]21] |
| 3 | 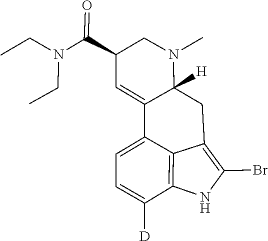 [SMILES: [2H]c1ccc2c3c(c(Br)[nH]c13)C[C@@H]1C2=C[C@@H](C(=O)N(CC)CC)CN1C] |
| 4 | 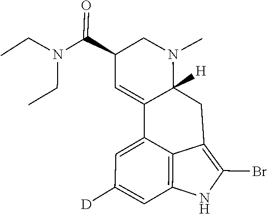 [SMILES: [2H]c1cc2c3c(c(Br)[nH]c3c1)C[C@@H]1C2=C[C@@H](C(=O)N(CC)CC)CN1C] |
| 5 | 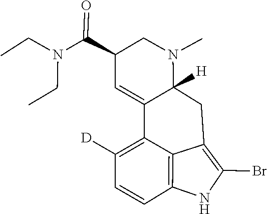 [SMILES: [2H]c1ccc2[nH]c(Br)c3c2c1C1=C[C@@H](C(=O)N(CC)CC)CN(C)[C@@H]1C3] |
| 6 | 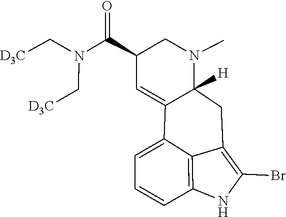 [SMILES: [2H]C([2H])([2H])CN(CC([2H])([2H])[2H])C(=O)[C@@H]1C=C2c3cccc4[nH]c(Br)c(c34)C[C@H]2N(C)C1] |
| 7 | 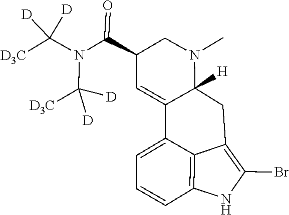 [SMILES: [2H]C([2H])([2H])C([2H])([2H])N(C(=O)[C@@H]1C=C2c3cccc4[nH]c(Br)c(c34)C[C@H]2N(C)C1)C([2H])([2H])C([2H])([2H])[2H]] |
| 8 | 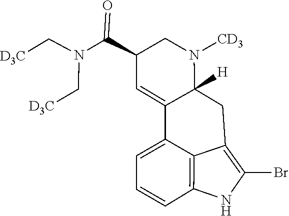 [SMILES: [2H]C([2H])([2H])CN(CC([2H])([2H])[2H])C(=O)[C@@H]1C=C2c3cccc4[nH]c(Br)c(c34)C[C@H]2N(C([2H])([2H])[2H])C1] |
| 9 | 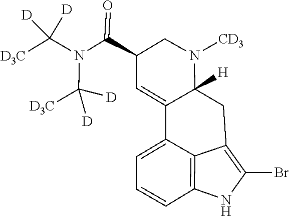 [SMILES: [2H]C([2H])([2H])N1C[C@H](C(=O)N(C([2H])([2H])C([2H])([2H])[2H])C([2H])([2H])C([2H])([2H])[2H])C=C2c3cccc4[nH]c(Br)c(c34)C[C@H]21] |
| 10 | 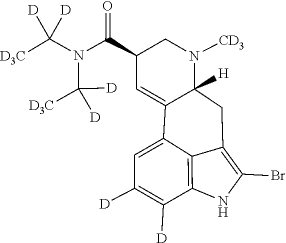 [SMILES: [2H]c1cc2c3c(c(Br)[nH]c3c1[2H])C[C@@H]1C2=C[C@@H](C(=O)N(C([2H])([2H])C([2H])([2H])[2H])C([2H])([2H])C([2H])([2H])[2H])CN1C([2H])([2H])[2H]] |
| 11 | 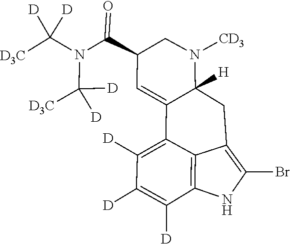 [SMILES: [2H]c1c([2H])c2c3c(c(Br)[nH]c3c1[2H])C[C@@H]1C2=C[C@@H](C(=O)N(C([2H])([2H])C([2H])([2H])[2H])C([2H])([2H])C([2H])([2H])[2H])CN1C([2H])([2H])[2H]] |
| 12 | 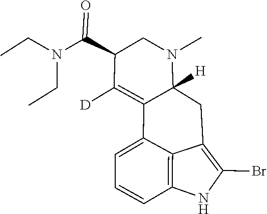 [SMILES: [2H]C1=C2c3cccc4[nH]c(Br)c(c34)C[C@H]2N(C)C[C@@H]1C(=O)N(CC)CC] |
| 13 | 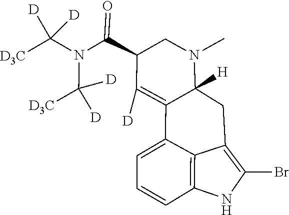 [SMILES: [2H]C1=C2c3cccc4[nH]c(Br)c(c34)C[C@H]2N(C)C[C@@H]1C(=O)N(C([2H])([2H])C([2H])([2H])[2H])C([2H])([2H])C([2H])([2H])[2H]] |
| 14 | 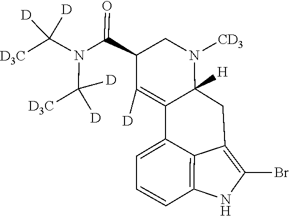 [SMILES: [2H]C1=C2c3cccc4[nH]c(Br)c(c34)C[C@H]2N(C([2H])([2H])[2H])C[C@@H]1C(=O)N(C([2H])([2H])C([2H])([2H])[2H])C([2H])([2H])C([2H])([2H])[2H]] |
| 15 | 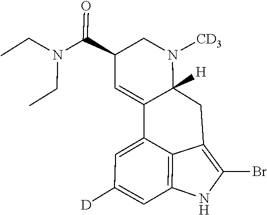 [SMILES: [2H]c1cc2c3c(c(Br)[nH]c3c1)C[C@@H]1C2=C[C@@H](C(=O)N(CC)CC)CN1C([2H])([2H])[2H]] |
| 16 | 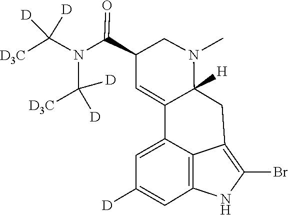 [SMILES: [2H]c1cc2c3c(c(Br)[nH]c3c1)C[C@@H]1C2=C[C@@H](C(=O)N(C([2H])([2H])C([2H])([2H])[2H])C([2H])([2H])C([2H])([2H])[2H])CN1C] |
| 17 | 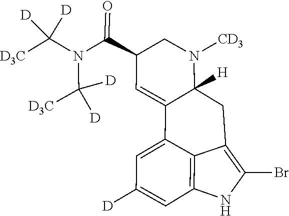 [SMILES: [2H]c1cc2c3c(c(Br)[nH]c3c1)C[C@@H]1C2=C[C@@H](C(=O)N(C([2H])([2H])C([2H])([2H])[2H])C([2H])([2H])C([2H])([2H])[2H])CN1C([2H])([2H])[2H]] |
| 18 | 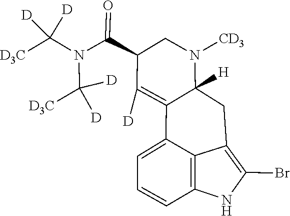 [SMILES: [2H]C1=C2c3cccc4[nH]c(Br)c(c34)C[C@H]2N(C([2H])([2H])[2H])C[C@@H]1C(=O)N(C([2H])([2H])C([2H])([2H])[2H])C([2H])([2H])C([2H])([2H])[2H]] |
| 19 | 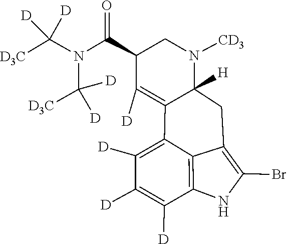 [SMILES: [2H]C1=C2c3c([2H])c([2H])c([2H])c4[nH]c(Br)c(c34)C[C@H]2N(C([2H])([2H])[2H])C[C@@H]1C(=O)N(C([2H])([2H])C([2H])([2H])[2H])C([2H])([2H])C([2H])([2H])[2H]] |
| 20 | 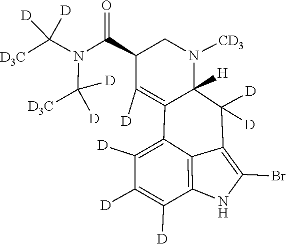 [SMILES: [2H]C1=C2c3c([2H])c([2H])c([2H])c4[nH]c(Br)c(c34)C([2H])([2H])[C@H]2N(C([2H])([2H])[2H])C[C@@H]1C(=O)N(C([2H])([2H])C([2H])([2H])[2H])C([2H])([2H])C([2H])([2H])[2H]] |
| 21 | 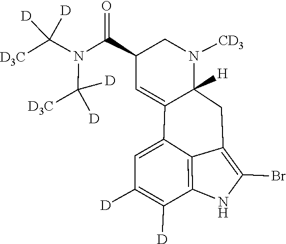 [SMILES: [2H]c1cc2c3c(c(Br)[nH]c3c1[2H])C[C@@H]1C2=C[C@@H](C(=O)N(C([2H])([2H])C([2H])([2H])[2H])C([2H])([2H])C([2H])([2H])[2H])CN1C([2H])([2H])[2H]] |
| 22 | 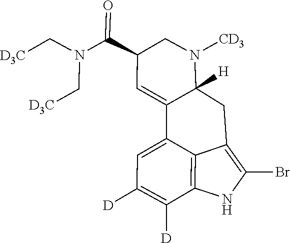 [SMILES: [2H]c1cc2c3c(c(Br)[nH]c3c1[2H])C[C@@H]1C2=C[C@@H](C(=O)N(CC([2H])([2H])[2H])CC([2H])([2H])[2H])CN1C([2H])([2H])[2H]] |
| 23 | 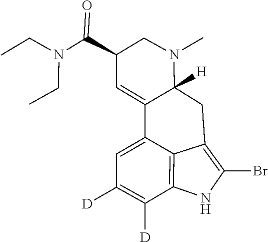 [SMILES: [2H]c1cc2c3c(c(Br)[nH]c3c1[2H])C[C@@H]1C2=C[C@@H](C(=O)N(CC)CC)CN1C] |
| 24 | 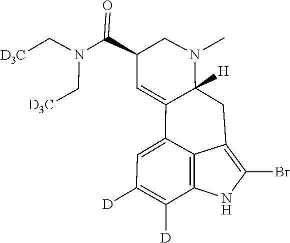 [SMILES: [2H]c1cc2c3c(c(Br)[nH]c3c1[2H])C[C@@H]1C2=C[C@@H](C(=O)N(CC([2H])([2H])[2H])CC([2H])([2H])[2H])CN1C] |
| 25 | 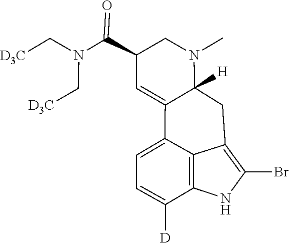 [SMILES: [2H]c1ccc2c3c(c(Br)[nH]c13)C[C@@H]1C2=C[C@@H](C(=O)N(CC([2H])([2H])[2H])CC([2H])([2H])[2H])CN1C] |
| 26 | 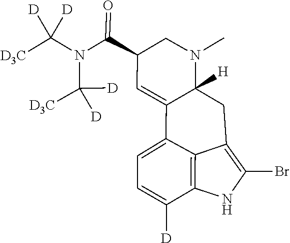 [SMILES: [2H]c1ccc2c3c(c(Br)[nH]c13)C[C@@H]1C2=C[C@@H](C(=O)N(C([2H])([2H])C([2H])([2H])[2H])C([2H])([2H])C([2H])([2H])[2H])CN1C] |
| 27 | 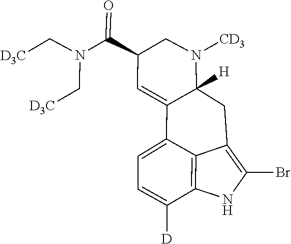 [SMILES: [2H]c1ccc2c3c(c(Br)[nH]c13)C[C@@H]1C2=C[C@@H](C(=O)N(CC([2H])([2H])[2H])CC([2H])([2H])[2H])CN1C([2H])([2H])[2H]] |
| 28 | 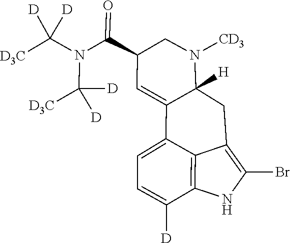 [SMILES: [2H]c1ccc2c3c(c(Br)[nH]c13)C[C@@H]1C2=C[C@@H](C(=O)N(C([2H])([2H])C([2H])([2H])[2H])C([2H])([2H])C([2H])([2H])[2H])CN1C([2H])([2H])[2H]] |
| 29 | 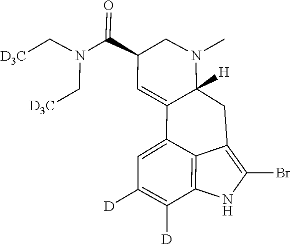 [SMILES: [2H]c1cc2c3c(c(Br)[nH]c3c1[2H])C[C@@H]1C2=C[C@@H](C(=O)N(CC([2H])([2H])[2H])CC([2H])([2H])[2H])CN1C] |
| 30 | 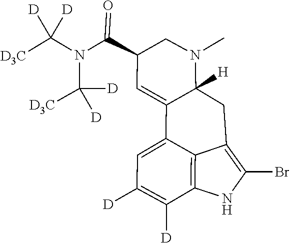 [SMILES: [2H]c1cc2c3c(c(Br)[nH]c3c1[2H])C[C@@H]1C2=C[C@@H](C(=O)N(C([2H])([2H])C([2H])([2H])[2H])C([2H])([2H])C([2H])([2H])[2H])CN1C] |
| 31 | 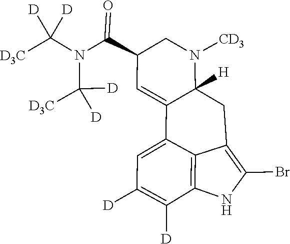 [SMILES: [2H]c1cc2c3c(c(Br)[nH]c3c1[2H])C[C@@H]1C2=C[C@@H](C(=O)N(C([2H])([2H])C([2H])([2H])[2H])C([2H])([2H])C([2H])([2H])[2H])CN1C([2H])([2H])[2H]] |
| 32 | 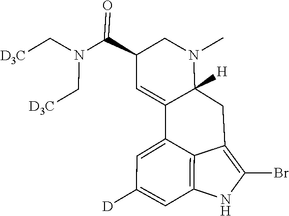 [SMILES: [2H]c1cc2c3c(c(Br)[nH]c3c1)C[C@@H]1C2=C[C@@H](C(=O)N(CC([2H])([2H])[2H])CC([2H])([2H])[2H])CN1C] |
| 33 | 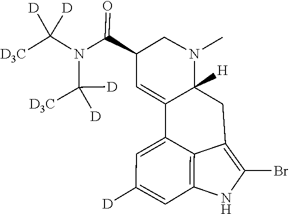 [SMILES: [2H]c1cc2c3c(c(Br)[nH]c3c1)C[C@@H]1C2=C[C@@H](C(=O)N(C([2H])([2H])C([2H])([2H])[2H])C([2H])([2H])C([2H])([2H])[2H])CN1C] |
| 34 | 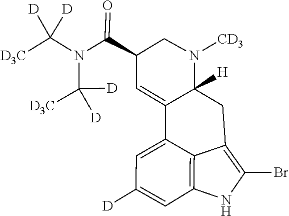 [SMILES: [2H]c1cc2c3c(c(Br)[nH]c3c1)C[C@@H]1C2=C[C@@H](C(=O)N(C([2H])([2H])C([2H])([2H])[2H])C([2H])([2H])C([2H])([2H])[2H])CN1C([2H])([2H])[2H]] |
| 35 | 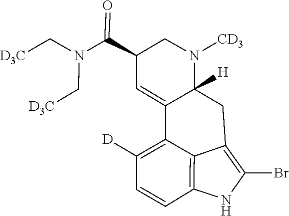 [SMILES: [2H]c1ccc2[nH]c(Br)c3c2c1C1=C[C@@H](C(=O)N(CC([2H])([2H])[2H])CC([2H])([2H])[2H])CN(C([2H])([2H])[2H])[C@@H]1C3] |
| 36 | 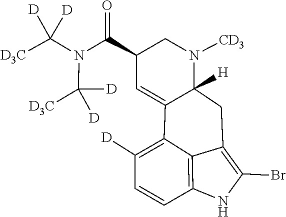 [SMILES: [2H]c1ccc2[nH]c(Br)c3c2c1C1=C[C@@H](C(=O)N(C([2H])([2H])C([2H])([2H])[2H])C([2H])([2H])C([2H])([2H])[2H])CN(C([2H])([2H])[2H])[C@@H]1C3] |
| 37 | 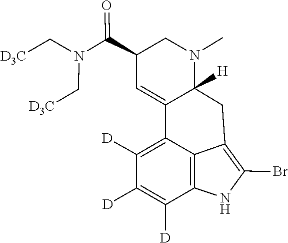 [SMILES: [2H]c1c([2H])c2c3c(c(Br)[nH]c3c1[2H])C[C@@H]1C2=C[C@@H](C(=O)N(CC([2H])([2H])[2H])CC([2H])([2H])[2H])CN1C] |
| 38 | 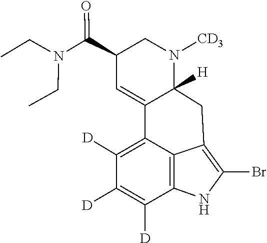 [SMILES: [2H]c1c([2H])c2c3c(c(Br)[nH]c3c1[2H])C[C@@H]1C2=C[C@@H](C(=O)N(CC)CC)CN1C([2H])([2H])[2H]] |
| 39 | 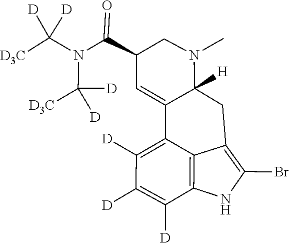 [SMILES: [2H]c1c([2H])c2c3c(c(Br)[nH]c3c1[2H])C[C@@H]1C2=C[C@@H](C(=O)N(C([2H])([2H])C([2H])([2H])[2H])C([2H])([2H])C([2H])([2H])[2H])CN1C] |
| 40 | 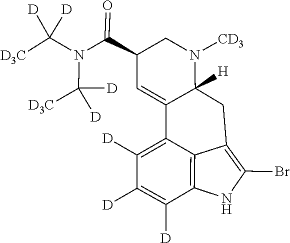 [SMILES: [2H]c1c([2H])c2c3c(c(Br)[nH]c3c1[2H])C[C@@H]1C2=C[C@@H](C(=O)N(C([2H])([2H])C([2H])([2H])[2H])C([2H])([2H])C([2H])([2H])[2H])CN1C([2H])([2H])[2H]] |
| 41 | 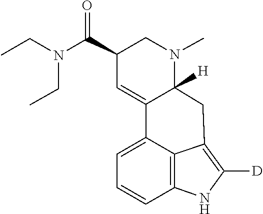 [SMILES: [2H]c1[nH]c2cccc3c2c1C[C@@H]1C3=C[C@@H](C(=O)N(CC)CC)CN1C] |
| 42 | 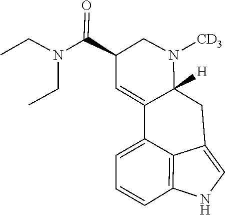 [SMILES: [2H]C([2H])([2H])N1C[C@H](C(=O)N(CC)CC)C=C2c3cccc4[nH]cc(c34)C[C@H]21] |
| 43 | 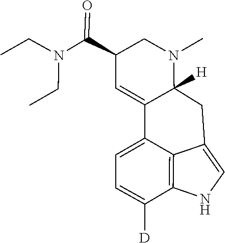 [SMILES: [2H]c1ccc2c3c(c[nH]c13)C[C@@H]1C2=C[C@@H](C(=O)N(CC)CC)CN1C] |
| 44 | 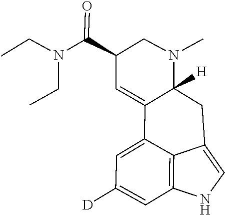 [SMILES: [2H]c1cc2c3c(c[nH]c3c1)C[C@@H]1C2=C[C@@H](C(=O)N(CC)CC)CN1C] |
| 45 | 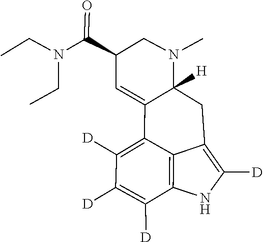 [SMILES: [2H]c1[nH]c2c([2H])c([2H])c([2H])c3c2c1C[C@@H]1C3=C[C@@H](C(=O)N(CC)CC)CN1C] |
| 46 | 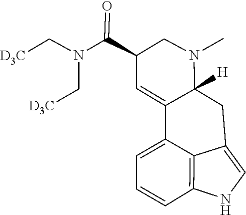 [SMILES: [2H]C([2H])([2H])CN(CC([2H])([2H])[2H])C(=O)[C@@H]1C=C2c3cccc4[nH]cc(c34)C[C@H]2N(C)C1] |
| 47 | 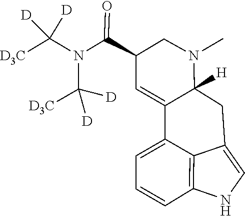 [SMILES: [2H]C([2H])([2H])C([2H])([2H])N(C(=O)[C@@H]1C=C2c3cccc4[nH]cc(c34)C[C@H]2N(C)C1)C([2H])([2H])C([2H])([2H])[2H]] |
| 48 | 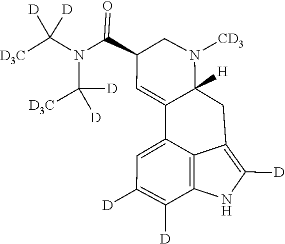 [SMILES: [2H]c1cc2c3c(c([2H])[nH]c3c1[2H])C[C@@H]1C2=C[C@@H](C(=O)N(C([2H])([2H])C([2H])([2H])[2H])C([2H])([2H])C([2H])([2H])[2H])CN1C([2H])([2H])[2H]] |
| 49 | 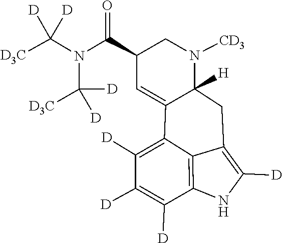 [SMILES: [2H]c1[nH]c2c([2H])c([2H])c([2H])c3c2c1C[C@@H]1C3=C[C@@H](C(=O)N(C([2H])([2H])C([2H])([2H])[2H])C([2H])([2H])C([2H])([2H])[2H])CN1C([2H])([2H])[2H]] |
| 50 | 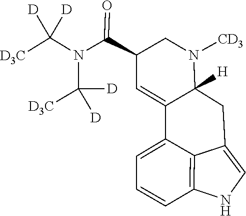 [SMILES: [2H]C([2H])([2H])N1C[C@H](C(=O)N(C([2H])([2H])C([2H])([2H])[2H])C([2H])([2H])C([2H])([2H])[2H])C=C2c3cccc4[nH]cc(c34)C[C@H]21] |
| 51 | 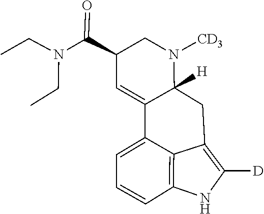 [SMILES: [2H]c1[nH]c2cccc3c2c1C[C@@H]1C3=C[C@@H](C(=O)N(CC)CC)CN1C([2H])([2H])[2H]] |
| 52 | 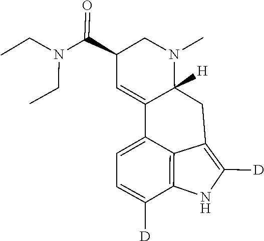 [SMILES: [2H]c1[nH]c2c([2H])ccc3c2c1C[C@@H]1C3=C[C@@H](C(=O)N(CC)CC)CN1C] |
| 53 | 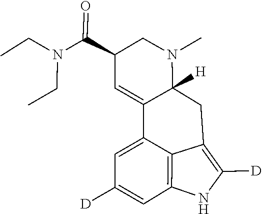 [SMILES: [2H]c1cc2c3c(c([2H])[nH]c3c1)C[C@@H]1C2=C[C@@H](C(=O)N(CC)CC)CN1C] |
| 54 | 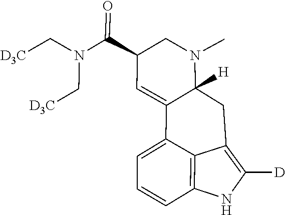 [SMILES: [2H]c1[nH]c2cccc3c2c1C[C@@H]1C3=C[C@@H](C(=O)N(CC([2H])([2H])[2H])CC([2H])([2H])[2H])CN1C] |
| 55 | 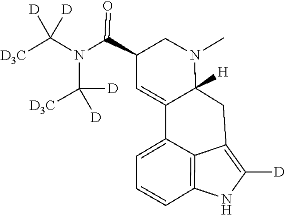 [SMILES: [2H]c1[nH]c2cccc3c2c1C[C@@H]1C3=C[C@@H](C(=O)N(C([2H])([2H])C([2H])([2H])[2H])C([2H])([2H])C([2H])([2H])[2H])CN1C] |
| 56 | 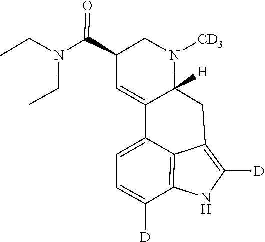 [SMILES: [2H]c1[nH]c2c([2H])ccc3c2c1C[C@@H]1C3=C[C@@H](C(=O)N(CC)CC)CN1C([2H])([2H])[2H]] |
| 57 | 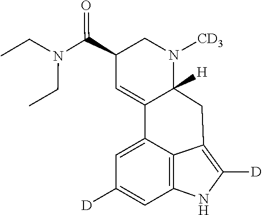 [SMILES: [2H]c1cc2c3c(c([2H])[nH]c3c1)C[C@@H]1C2=C[C@@H](C(=O)N(CC)CC)CN1C([2H])([2H])[2H]] |
| 58 | 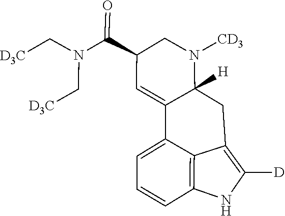 [SMILES: [2H]c1[nH]c2cccc3c2c1C[C@@H]1C3=C[C@@H](C(=O)N(CC([2H])([2H])[2H])CC([2H])([2H])[2H])CN1C([2H])([2H])[2H]] |
| 59 | 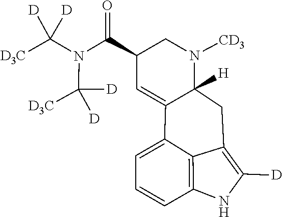 [SMILES: [2H]c1[nH]c2cccc3c2c1C[C@@H]1C3=C[C@@H](C(=O)N(C([2H])([2H])C([2H])([2H])[2H])C([2H])([2H])C([2H])([2H])[2H])CN1C([2H])([2H])[2H]] |
| 60 | 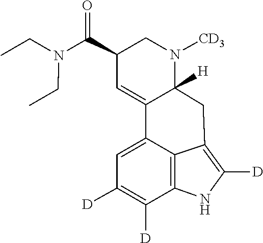 [SMILES: [2H]c1cc2c3c(c([2H])[nH]c3c1[2H])C[C@@H]1C2=C[C@@H](C(=O)N(CC)CC)CN1C([2H])([2H])[2H]] |
| 61 | 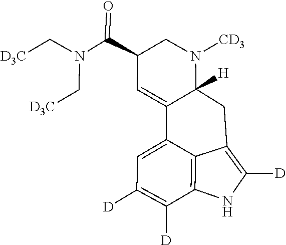 [SMILES: [2H]c1cc2c3c(c([2H])[nH]c3c1[2H])C[C@@H]1C2=C[C@@H](C(=O)N(CC([2H])([2H])[2H])CC([2H])([2H])[2H])CN1C([2H])([2H])[2H]] |
| 62 | 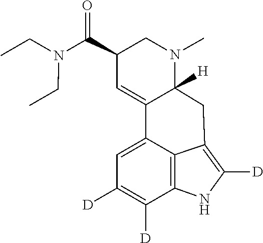 [SMILES: [2H]c1cc2c3c(c([2H])[nH]c3c1[2H])C[C@@H]1C2=C[C@@H](C(=O)N(CC)CC)CN1C] |
| 63 | 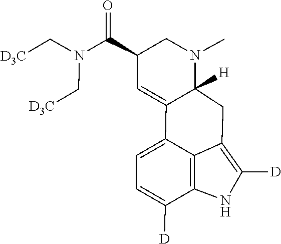 [SMILES: [2H]c1[nH]c2c([2H])ccc3c2c1C[C@@H]1C3=C[C@@H](C(=O)N(CC([2H])([2H])[2H])CC([2H])([2H])[2H])CN1C] |
| 64 | 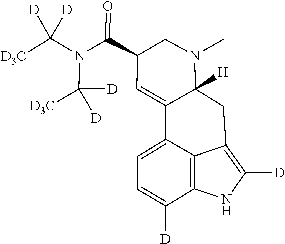 [SMILES: [2H]c1[nH]c2c([2H])ccc3c2c1C[C@@H]1C3=C[C@@H](C(=O)N(C([2H])([2H])C([2H])([2H])[2H])C([2H])([2H])C([2H])([2H])[2H])CN1C] |
| 65 | 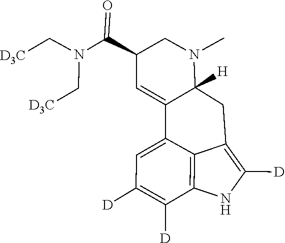 [SMILES: [2H]c1cc2c3c(c([2H])[nH]c3c1[2H])C[C@@H]1C2=C[C@@H](C(=O)N(CC([2H])([2H])[2H])CC([2H])([2H])[2H])CN1C] |
| 66 | 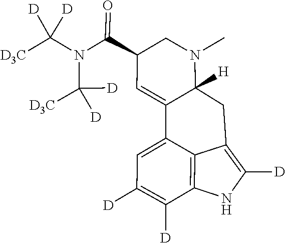 [SMILES: [2H]c1cc2c3c(c([2H])[nH]c3c1[2H])C[C@@H]1C2=C[C@@H](C(=O)N(C([2H])([2H])C([2H])([2H])[2H])C([2H])([2H])C([2H])([2H])[2H])CN1C] |
| 67 | 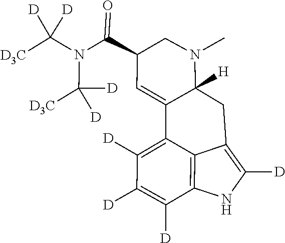 [SMILES: [2H]c1[nH]c2c([2H])c([2H])c([2H])c3c2c1C[C@@H]1C3=C[C@@H](C(=O)N(C([2H])([2H])C([2H])([2H])[2H])C([2H])([2H])C([2H])([2H])[2H])CN1C] |
| 68 | 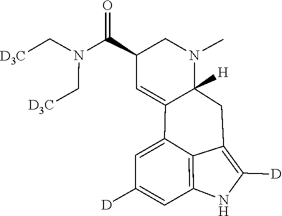 [SMILES: [2H]c1cc2c3c(c([2H])[nH]c3c1)C[C@@H]1C2=C[C@@H](C(=O)N(CC([2H])([2H])[2H])CC([2H])([2H])[2H])CN1C] |
| 69 | 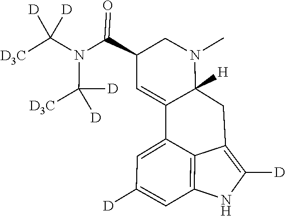 [SMILES: [2H]c1cc2c3c(c([2H])[nH]c3c1)C[C@@H]1C2=C[C@@H](C(=O)N(C([2H])([2H])C([2H])([2H])[2H])C([2H])([2H])C([2H])([2H])[2H])CN1C] |
| 70 | 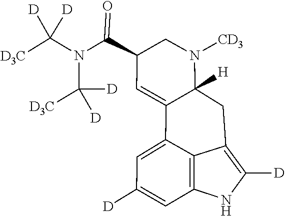 [SMILES: [2H]c1cc2c3c(c([2H])[nH]c3c1)C[C@@H]1C2=C[C@@H](C(=O)N(C([2H])([2H])C([2H])([2H])[2H])C([2H])([2H])C([2H])([2H])[2H])CN1C([2H])([2H])[2H]] |
| 71 | 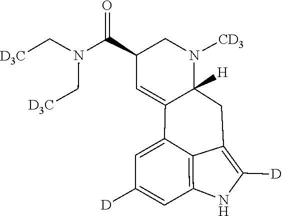 [SMILES: [2H]c1cc2c3c(c([2H])[nH]c3c1)C[C@@H]1C2=C[C@@H](C(=O)N(CC([2H])([2H])[2H])CC([2H])([2H])[2H])CN1C([2H])([2H])[2H]] |
| 72 | 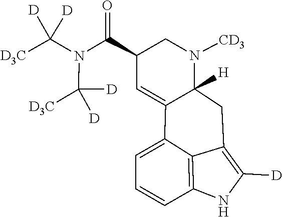 [SMILES: [2H]c1[nH]c2cccc3c2c1C[C@@H]1C3=C[C@@H](C(=O)N(C([2H])([2H])C([2H])([2H])[2H])C([2H])([2H])C([2H])([2H])[2H])CN1C([2H])([2H])[2H]] |
| 73 | 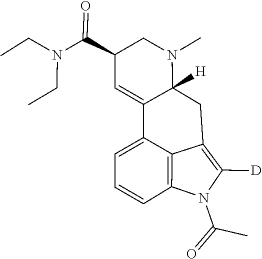 [SMILES: [2H]c1c2c3c(cccc3n1C(C)=O)C1=C[C@@H](C(=O)N(CC)CC)CN(C)[C@@H]1C2] |
| 74 | 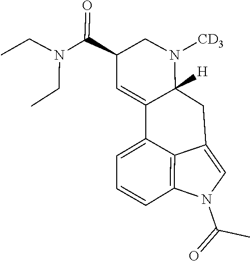 [SMILES: [2H]C([2H])([2H])N1C[C@H](C(=O)N(CC)CC)C=C2c3cccc4c3c(cn4C(C)=O)C[C@H]21] |
| 75 | 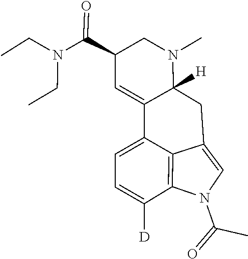 [SMILES: [2H]c1ccc2c3c(cn(C(C)=O)c13)C[C@@H]1C2=C[C@@H](C(=O)N(CC)CC)CN1C] |
| 76 | 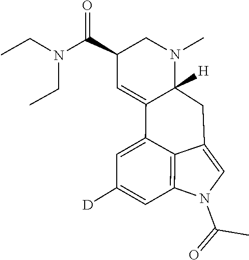 [SMILES: [2H]c1cc2c3c(cn(C(C)=O)c3c1)C[C@@H]1C2=C[C@@H](C(=O)N(CC)CC)CN1C] |
| 77 | 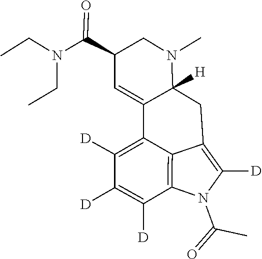 [SMILES: [2H]c1c([2H])c([2H])c2c3c(c([2H])n2C(C)=O)C[C@@H]2C(=C[C@@H](C(=O)N(CC)CC)CN2C)c13] |
| 78 | 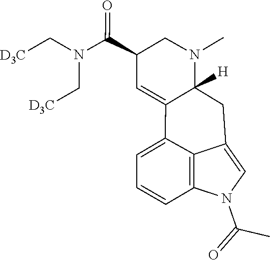 [SMILES: [2H]C([2H])([2H])CN(CC([2H])([2H])[2H])C(=O)[C@@H]1C=C2c3cccc4c3c(cn4C(C)=O)C[C@H]2N(C)C1] |
| 79 | 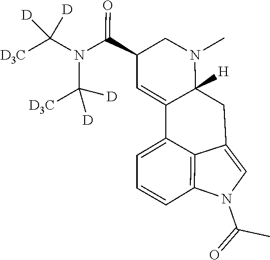 [SMILES: [2H]C([2H])([2H])C([2H])([2H])N(C(=O)[C@@H]1C=C2c3cccc4c3c(cn4C(C)=O)C[C@H]2N(C)C1)C([2H])([2H])C([2H])([2H])[2H]] |
| 80 | 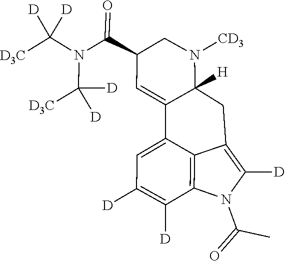 [SMILES: [2H]c1cc2c3c(c([2H])n(C(C)=O)c3c1[2H])C[C@@H]1C2=C[C@@H](C(=O)N(C([2H])([2H])C([2H])([2H])[2H])C([2H])([2H])C([2H])([2H])[2H])CN1C([2H])([2H])[2H]] |
| 81 | 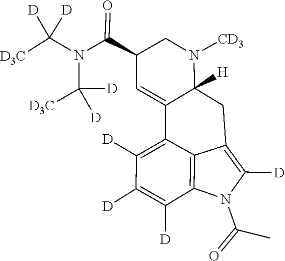 [SMILES: [2H]c1c([2H])c([2H])c2c3c(c([2H])n2C(C)=O)C[C@@H]2C(=C[C@@H](C(=O)N(C([2H])([2H])C([2H])([2H])[2H])C([2H])([2H])C([2H])([2H])[2H])CN2C([2H])([2H])[2H])c13] |
| 82 | 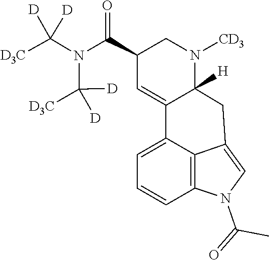 [SMILES: [2H]C([2H])([2H])N1C[C@H](C(=O)N(C([2H])([2H])C([2H])([2H])[2H])C([2H])([2H])C([2H])([2H])[2H])C=C2c3cccc4c3c(cn4C(C)=O)C[C@H]21] |
| 83 | 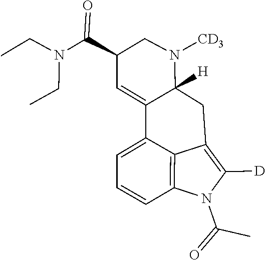 [SMILES: [2H]c1c2c3c(cccc3n1C(C)=O)C1=C[C@@H](C(=O)N(CC)CC)CN(C([2H])([2H])[2H])[C@@H]1C2] |
| 84 | 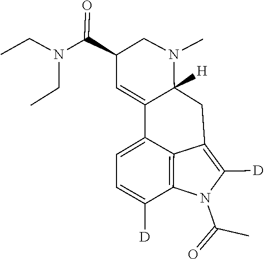 [SMILES: [2H]c1ccc2c3c(c([2H])n(C(C)=O)c13)C[C@@H]1C2=C[C@@H](C(=O)N(CC)CC)CN1C] |
| 85 | 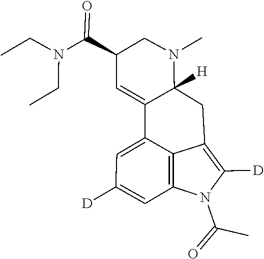 [SMILES: [2H]c1cc2c3c(c([2H])n(C(C)=O)c3c1)C[C@@H]1C2=C[C@@H](C(=O)N(CC)CC)CN1C] |
| 86 | 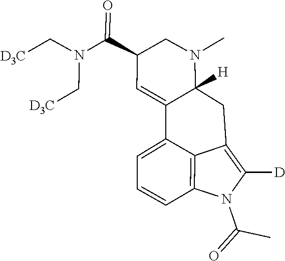 [SMILES: [2H]c1c2c3c(cccc3n1C(C)=O)C1=C[C@@H](C(=O)N(CC([2H])([2H])[2H])CC([2H])([2H])[2H])CN(C)[C@@H]1C2] |
| 87 | 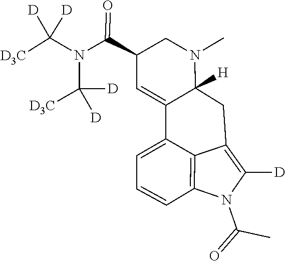 [SMILES: [2H]c1c2c3c(cccc3n1C(C)=O)C1=C[C@@H](C(=O)N(C([2H])([2H])C([2H])([2H])[2H])C([2H])([2H])C([2H])([2H])[2H])CN(C)[C@@H]1C2] |
| 88 | 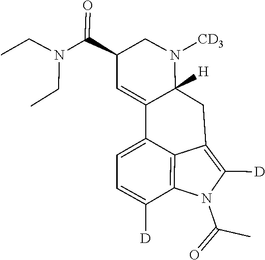 [SMILES: [2H]c1ccc2c3c(c([2H])n(C(C)=O)c13)C[C@@H]1C2=C[C@@H](C(=O)N(CC)CC)CN1C([2H])([2H])[2H]] |
| 89 | 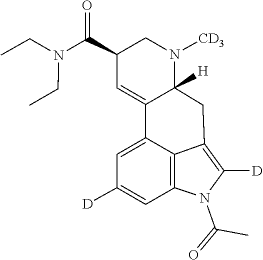 [SMILES: [2H]c1cc2c3c(c([2H])n(C(C)=O)c3c1)C[C@@H]1C2=C[C@@H](C(=O)N(CC)CC)CN1C([2H])([2H])[2H]] |
| 90 | 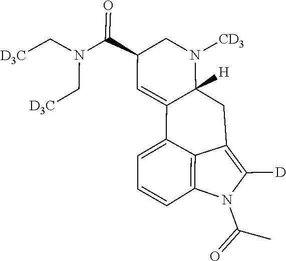 [SMILES: [2H]c1c2c3c(cccc3n1C(C)=O)C1=C[C@@H](C(=O)N(CC([2H])([2H])[2H])CC([2H])([2H])[2H])CN(C([2H])([2H])[2H])[C@@H]1C2] |
| 91 | 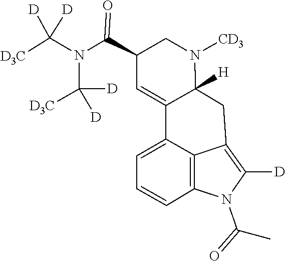 [SMILES: [2H]c1c2c3c(cccc3n1C(C)=O)C1=C[C@@H](C(=O)N(C([2H])([2H])C([2H])([2H])[2H])C([2H])([2H])C([2H])([2H])[2H])CN(C([2H])([2H])[2H])[C@@H]1C2] |
| 92 | 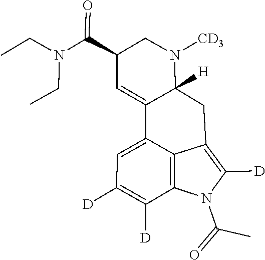 [SMILES: [2H]c1cc2c3c(c([2H])n(C(C)=O)c3c1[2H])C[C@@H]1C2=C[C@@H](C(=O)N(CC)CC)CN1C([2H])([2H])[2H]] |
| 93 | 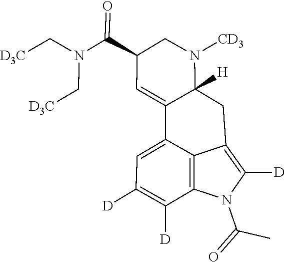 [SMILES: [2H]c1cc2c3c(c([2H])n(C(C)=O)c3c1[2H])C[C@@H]1C2=C[C@@H](C(=O)N(CC([2H])([2H])[2H])CC([2H])([2H])[2H])CN1C([2H])([2H])[2H]] |
| 94 | 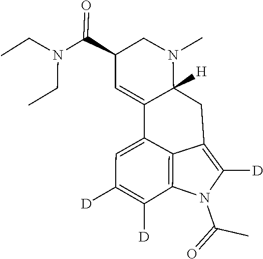 [SMILES: [2H]c1cc2c3c(c([2H])n(C(C)=O)c3c1[2H])C[C@@H]1C2=C[C@@H](C(=O)N(CC)CC)CN1C] |
| 95 | 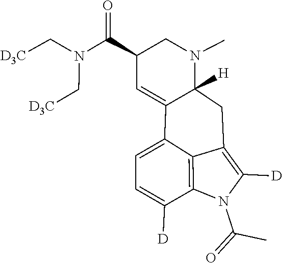 [SMILES: [2H]c1ccc2c3c(c([2H])n(C(C)=O)c13)C[C@@H]1C2=C[C@@H](C(=O)N(CC([2H])([2H])[2H])CC([2H])([2H])[2H])CN1C] |
| 96 | 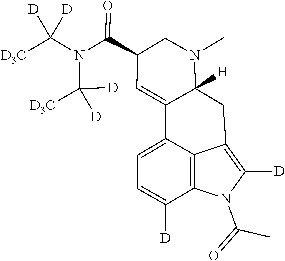 [SMILES: [2H]c1ccc2c3c(c([2H])n(C(C)=O)c13)C[C@@H]1C2=C[C@@H](C(=O)N(C([2H])([2H])C([2H])([2H])[2H])C([2H])([2H])C([2H])([2H])[2H])CN1C] |
| 97 | 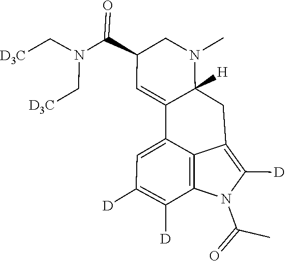 [SMILES: [2H]c1cc2c3c(c([2H])n(C(C)=O)c3c1[2H])C[C@@H]1C2=C[C@@H](C(=O)N(CC([2H])([2H])[2H])CC([2H])([2H])[2H])CN1C] |
| 98 | 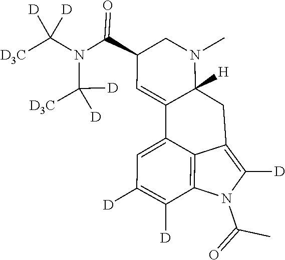 [SMILES: [2H]c1cc2c3c(c([2H])n(C(C)=O)c3c1[2H])C[C@@H]1C2=C[C@@H](C(=O)N(C([2H])([2H])C([2H])([2H])[2H])C([2H])([2H])C([2H])([2H])[2H])CN1C] |
| 99 | 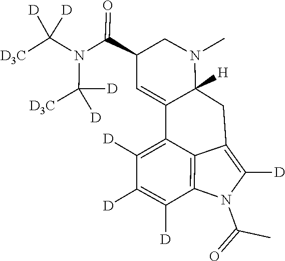 [SMILES: [2H]c1c([2H])c([2H])c2c3c(c([2H])n2C(C)=O)C[C@@H]2C(=C[C@@H](C(=O)N(C([2H])([2H])C([2H])([2H])[2H])C([2H])([2H])C([2H])([2H])[2H])CN2C)c13] |
| 100 | 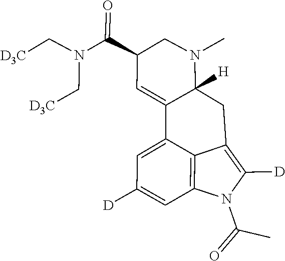 [SMILES: [2H]c1cc2c3c(c([2H])n(C(C)=O)c3c1)C[C@@H]1C2=C[C@@H](C(=O)N(CC([2H])([2H])[2H])CC([2H])([2H])[2H])CN1C] |
| 101 | 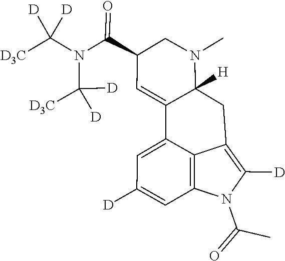 [SMILES: [2H]c1cc2c3c(c([2H])n(C(C)=O)c3c1)C[C@@H]1C2=C[C@@H](C(=O)N(C([2H])([2H])C([2H])([2H])[2H])C([2H])([2H])C([2H])([2H])[2H])CN1C] |
| 102 | 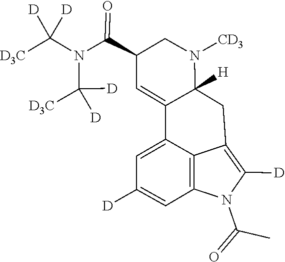 [SMILES: [2H]c1cc2c3c(c([2H])n(C(C)=O)c3c1)C[C@@H]1C2=C[C@@H](C(=O)N(C([2H])([2H])C([2H])([2H])[2H])C([2H])([2H])C([2H])([2H])[2H])CN1C([2H])([2H])[2H]] |
| 103 | 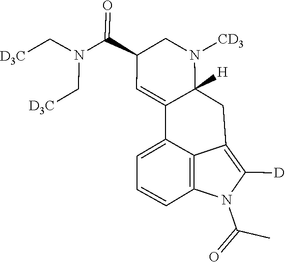 [SMILES: [2H]c1c2c3c(cccc3n1C(C)=O)C1=C[C@@H](C(=O)N(CC([2H])([2H])[2H])CC([2H])([2H])[2H])CN(C([2H])([2H])[2H])[C@@H]1C2] |
| 104 | 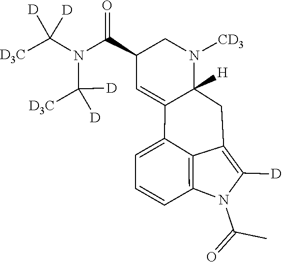 [SMILES: [2H]c1c2c3c(cccc3n1C(C)=O)C1=C[C@@H](C(=O)N(C([2H])([2H])C([2H])([2H])[2H])C([2H])([2H])C([2H])([2H])[2H])CN(C([2H])([2H])[2H])[C@@H]1C2] |
| 105 | 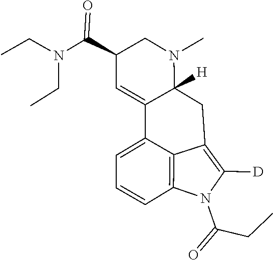 [SMILES: [2H]c1c2c3c(cccc3n1C(=O)CC)C1=C[C@@H](C(=O)N(CC)CC)CN(C)[C@@H]1C2] |
| 106 | 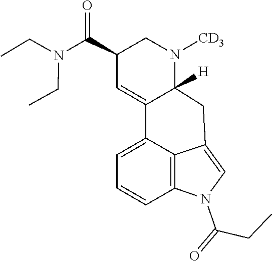 [SMILES: [2H]C([2H])([2H])N1C[C@H](C(=O)N(CC)CC)C=C2c3cccc4c3c(cn4C(=O)CC)C[C@H]21] |
| 107 | 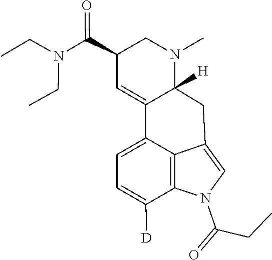 [SMILES: [2H]c1ccc2c3c(cn(C(=O)CC)c13)C[C@@H]1C2=C[C@@H](C(=O)N(CC)CC)CN1C] |
| 108 | 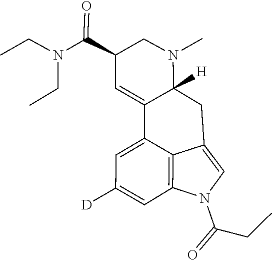 [SMILES: [2H]c1cc2c3c(cn(C(=O)CC)c3c1)C[C@@H]1C2=C[C@@H](C(=O)N(CC)CC)CN1C] |
| 109 | 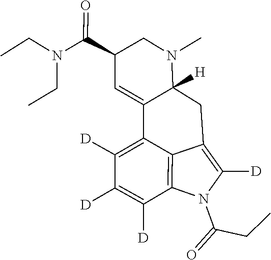 [SMILES: [2H]c1c([2H])c([2H])c2c3c(c([2H])n2C(=O)CC)C[C@@H]2C(=C[C@@H](C(=O)N(CC)CC)CN2C)c13] |
| 110 | 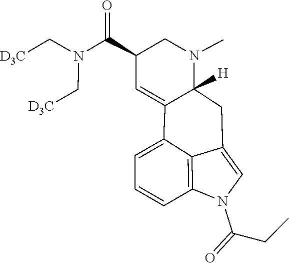 [SMILES: [2H]C([2H])([2H])CN(CC([2H])([2H])[2H])C(=O)[C@@H]1C=C2c3cccc4c3c(cn4C(=O)CC)C[C@H]2N(C)C1] |
| 111 | 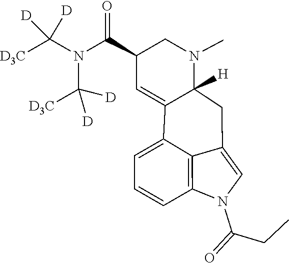 [SMILES: [2H]C([2H])([2H])C([2H])([2H])N(C(=O)[C@@H]1C=C2c3cccc4c3c(cn4C(=O)CC)C[C@H]2N(C)C1)C([2H])([2H])C([2H])([2H])[2H]] |
| 112 | 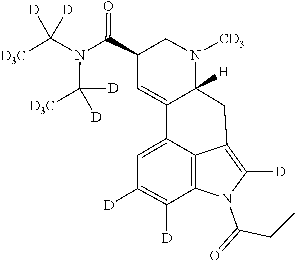 [SMILES: [2H]c1cc2c3c(c([2H])n(C(=O)CC)c3c1[2H])C[C@@H]1C2=C[C@@H](C(=O)N(C([2H])([2H])C([2H])([2H])[2H])C([2H])([2H])C([2H])([2H])[2H])CN1C([2H])([2H])[2H]] |
| 113 | 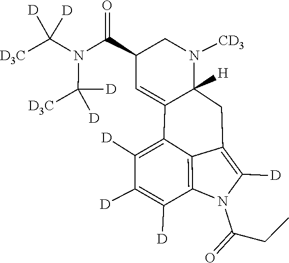 [SMILES: [2H]c1c([2H])c([2H])c2c3c(c([2H])n2C(=O)CC)C[C@@H]2C(=C[C@@H](C(=O)N(C([2H])([2H])C([2H])([2H])[2H])C([2H])([2H])C([2H])([2H])[2H])CN2C([2H])([2H])[2H])c13] |
| 114 | 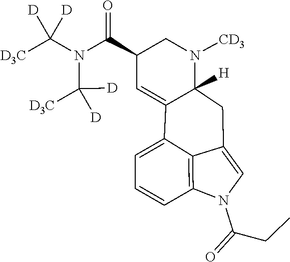 [SMILES: [2H]C([2H])([2H])N1C[C@H](C(=O)N(C([2H])([2H])C([2H])([2H])[2H])C([2H])([2H])C([2H])([2H])[2H])C=C2c3cccc4c3c(cn4C(=O)CC)C[C@H]21] |
| 115 | 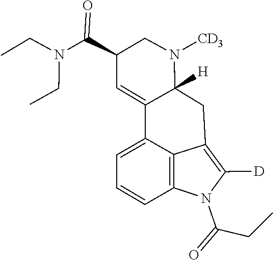 [SMILES: [2H]c1c2c3c(cccc3n1C(=O)CC)C1=C[C@@H](C(=O)N(CC)CC)CN(C([2H])([2H])[2H])[C@@H]1C2] |
| 116 | 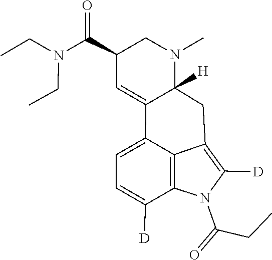 [SMILES: [2H]c1ccc2c3c(c([2H])n(C(=O)CC)c13)C[C@@H]1C2=C[C@@H](C(=O)N(CC)CC)CN1C] |
| 117 | 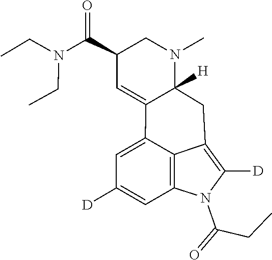 [SMILES: [2H]c1cc2c3c(c([2H])n(C(=O)CC)c3c1)C[C@@H]1C2=C[C@@H](C(=O)N(CC)CC)CN1C] |
| 118 | 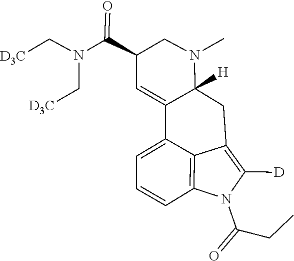 [SMILES: [2H]c1c2c3c(cccc3n1C(=O)CC)C1=C[C@@H](C(=O)N(CC([2H])([2H])[2H])CC([2H])([2H])[2H])CN(C)[C@@H]1C2] |
| 119 | 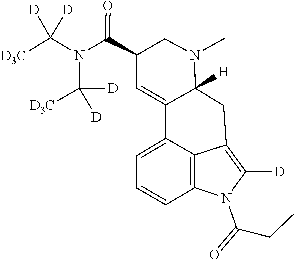 [SMILES: [2H]c1c2c3c(cccc3n1C(=O)CC)C1=C[C@@H](C(=O)N(C([2H])([2H])C([2H])([2H])[2H])C([2H])([2H])C([2H])([2H])[2H])CN(C)[C@@H]1C2] |
| 120 | 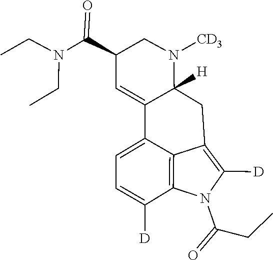 [SMILES: [2H]c1ccc2c3c(c([2H])n(C(=O)CC)c13)C[C@@H]1C2=C[C@@H](C(=O)N(CC)CC)CN1C([2H])([2H])[2H]] |
| 121 | 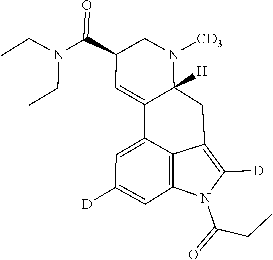 [SMILES: [2H]c1cc2c3c(c([2H])n(C(=O)CC)c3c1)C[C@@H]1C2=C[C@@H](C(=O)N(CC)CC)CN1C([2H])([2H])[2H]] |
| 122 | 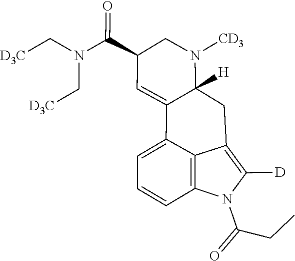 [SMILES: [2H]c1c2c3c(cccc3n1C(=O)CC)C1=C[C@@H](C(=O)N(CC([2H])([2H])[2H])CC([2H])([2H])[2H])CN(C([2H])([2H])[2H])[C@@H]1C2] |
| 123 | 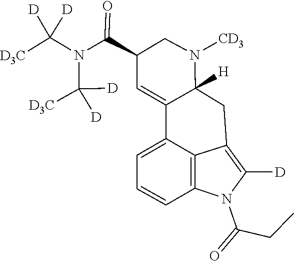 [SMILES: [2H]c1c2c3c(cccc3n1C(=O)CC)C1=C[C@@H](C(=O)N(C([2H])([2H])C([2H])([2H])[2H])C([2H])([2H])C([2H])([2H])[2H])CN(C([2H])([2H])[2H])[C@@H]1C2] |
| 124 | 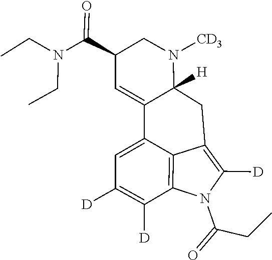 [SMILES: [2H]c1cc2c3c(c([2H])n(C(=O)CC)c3c1[2H])C[C@@H]1C2=C[C@@H](C(=O)N(CC)CC)CN1C([2H])([2H])[2H]] |
| 125 | 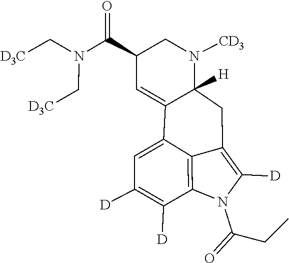 [SMILES: [2H]c1cc2c3c(c([2H])n(C(=O)CC)c3c1[2H])C[C@@H]1C2=C[C@@H](C(=O)N(CC([2H])([2H])[2H])CC([2H])([2H])[2H])CN1C([2H])([2H])[2H]] |
| 126 | 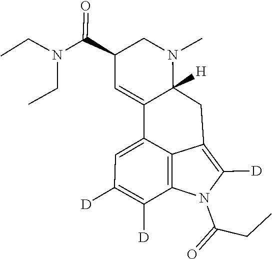 [SMILES: [2H]c1cc2c3c(c([2H])n(C(=O)CC)c3c1[2H])C[C@@H]1C2=C[C@@H](C(=O)N(CC)CC)CN1C] |
| 127 | 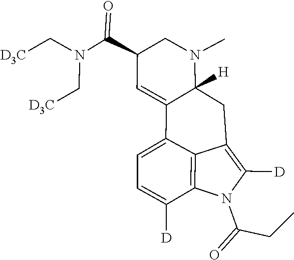 [SMILES: [2H]c1ccc2c3c(c([2H])n(C(=O)CC)c13)C[C@@H]1C2=C[C@@H](C(=O)N(CC([2H])([2H])[2H])CC([2H])([2H])[2H])CN1C] |
| 128 | 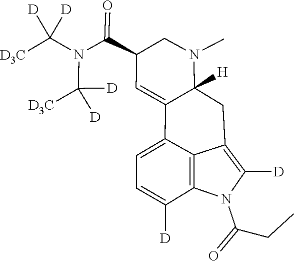 [SMILES: [2H]c1ccc2c3c(c([2H])n(C(=O)CC)c13)C[C@@H]1C2=C[C@@H](C(=O)N(C([2H])([2H])C([2H])([2H])[2H])C([2H])([2H])C([2H])([2H])[2H])CN1C] |
| 129 | 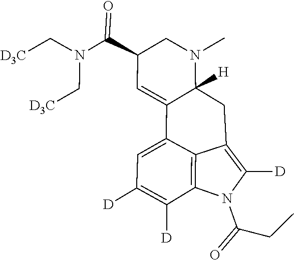 [SMILES: [2H]c1cc2c3c(c([2H])n(C(=O)CC)c3c1[2H])C[C@@H]1C2=C[C@@H](C(=O)N(CC([2H])([2H])[2H])CC([2H])([2H])[2H])CN1C] |
| 130 | 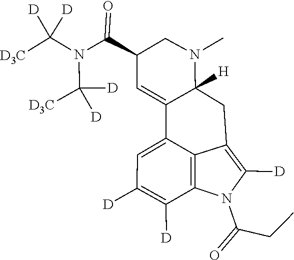 [SMILES: [2H]c1cc2c3c(c([2H])n(C(=O)CC)c3c1[2H])C[C@@H]1C2=C[C@@H](C(=O)N(C([2H])([2H])C([2H])([2H])[2H])C([2H])([2H])C([2H])([2H])[2H])CN1C] |
| 131 | 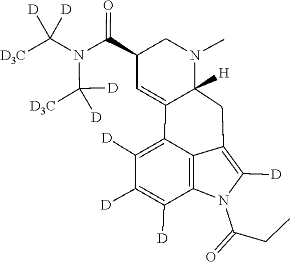 [SMILES: [2H]c1c([2H])c([2H])c2c3c(c([2H])n2C(=O)CC)C[C@@H]2C(=C[C@@H](C(=O)N(C([2H])([2H])C([2H])([2H])[2H])C([2H])([2H])C([2H])([2H])[2H])CN2C)c13] |
| 132 | 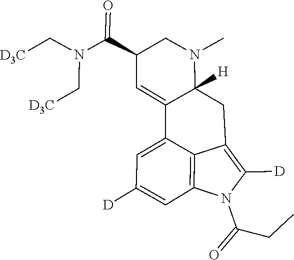 [SMILES: [2H]c1cc2c3c(c([2H])n(C(=O)CC)c3c1)C[C@@H]1C2=C[C@@H](C(=O)N(CC([2H])([2H])[2H])CC([2H])([2H])[2H])CN1C] |
| 133 | 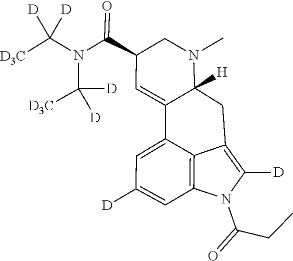 [SMILES: [2H]c1cc2c3c(c([2H])n(C(=O)CC)c3c1)C[C@@H]1C2=C[C@@H](C(=O)N(C([2H])([2H])C([2H])([2H])[2H])C([2H])([2H])C([2H])([2H])[2H])CN1C] |
| 134 | 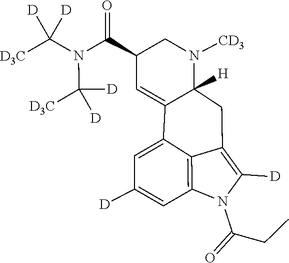 [SMILES: [2H]c1cc2c3c(c([2H])n(C(=O)CC)c3c1)C[C@@H]1C2=C[C@@H](C(=O)N(C([2H])([2H])C([2H])([2H])[2H])C([2H])([2H])C([2H])([2H])[2H])CN1C([2H])([2H])[2H]] |
| 135 | 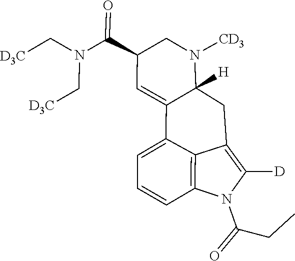 [SMILES: [2H]c1c2c3c(cccc3n1C(=O)CC)C1=C[C@@H](C(=O)N(CC([2H])([2H])[2H])CC([2H])([2H])[2H])CN(C([2H])([2H])[2H])[C@@H]1C2] |
| 136 | 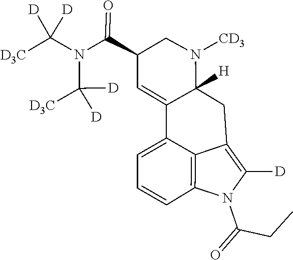 [SMILES: [2H]c1c2c3c(cccc3n1C(=O)CC)C1=C[C@@H](C(=O)N(C([2H])([2H])C([2H])([2H])[2H])C([2H])([2H])C([2H])([2H])[2H])CN(C([2H])([2H])[2H])[C@@H]1C2] |

The compounds of the present invention can also be in salt forms, such as acid or base salts of the compounds of the present invention. Illustrative examples of pharmaceutically acceptable acid salts are mineral acid (hydrochloric acid, hydrobromic acid, phosphoric acid, and the like) salts, organic acid (fumaric acid, acetic acid, propionic acid, glutamic acid, citric acid, tartaric acid and the like) salts. It is understood that the pharmaceutically acceptable salts are non-toxic. Additional information on suitable pharmaceutically acceptable salts can be found in Remington's Pharmaceutical Sciences, 17th ed., Mack Publishing Company, Easton, Pa., 1985, which is incorporated herein by reference.

The present invention includes all tautomers and stereoisomers of compounds of the formulas above, including compounds of Formulas (A), (I), (II), (IIa), (IIb), (IIc), and Table 1, either in admixture or in pure or substantially pure form. The compounds of the present invention can have asymmetric centers at the carbon atoms, and therefore the compounds of the present invention can exist in diastereomeric or enantiomeric forms or mixtures thereof. All conformational isomers (e.g., cis and trans isomers) and all optical isomers (e.g., enantiomers and diastereomers), racemic, diastereomeric and other mixtures of such isomers.

In addition, all physical forms of the compounds of the Formulas above are intended herein, including the compounds of Formulas (A), (I), (II), (IIa), (IIb), (IIc), and Table 1, in the form of solvates, such as hydrates. Moreover, non-crystalline and crystalline forms of the compounds of Formulas (A), (I), (II), (IIa), (IIb), (IIc), and Table 1, including amorphous forms, isomorphs and polymorphs are within the scope of the present invention.

Exemplary compounds according to the present invention are chiral. Such compounds can be prepared as is known to those of skill in the art can be prepared as single enantiomers, or enantiomerically enriched mixtures, or racemic mixtures as contemplated herein; such compounds having more than one stereocenter can also be prepared as diastereomeric, enantiomeric or racemic mixtures as contemplated herein. Furthermore, diastereomer and enantiomer products can be separated by chromatography, fractional crystallization or other methods known to those of skill in the art.

### Pharmaceutical Compositions and Formulations

In some embodiments, the present invention provides a pharmaceutical composition comprising a compound of the present invention, such as a composition comprising a compound of Formulas (A), (I), (II), (IIa), (IIb), (IIc), and Table 1, illustrated above, and a pharmaceutically acceptable excipient. Such compositions are suitable for administration to a subject, such as a human subject.

The presently disclosed pharmaceutical compositions can be prepared in a wide variety of oral, parenteral and topical dosage forms. Oral preparations include tablets, pills, powder, capsules, liquids, lozenges, cachets, gels, syrups, slurries, suspensions, etc., suitable for ingestion by the patient. The compositions of the present invention can also be administered by injection, that is, intravenously, intramuscularly, intracutaneously, subcutaneously, intraduodenally, or intraperitoneally. Also, the compositions described herein can be administered by inhalation, for example, intranasally. Additionally, the compositions of the present invention can be administered transdermally. The compositions of this invention can also be administered by intraocular, intravaginal, and intrarectal routes including suppositories, insufflation, powders and aerosol formulations (for examples of steroid inhalants, see Rohatagi, J. Clin. Pharmacol. 35:1187-1193, 1995; Tjwa, Ann. Allergy Asthma Immunol. 75:107-111, 1995). Accordingly, the present invention also provides pharmaceutical compositions including a pharmaceutically acceptable carrier or excipient and the compounds of the present invention.

For preparing pharmaceutical compositions from the compounds disclosed herein, pharmaceutically acceptable carriers can be either solid or liquid. Solid form preparations include powders, tablets, pills, capsules, cachets, suppositories, and dispersible granules. A solid carrier can be one or more substances, which may also act as diluents, flavoring agents, binders, preservatives, tablet disintegrating agents, or an encapsulating material. Details on techniques for formulation and administration are well described in the scientific and patent literature, see, e.g., the latest edition of Remington's Pharmaceutical Sciences, Mack Publishing Co, Easton PA (“Remington's”).

In powders, the carrier is a finely divided solid, which is in a mixture with the finely divided active component. In tablets, the active component is mixed with the carrier having the necessary binding properties in suitable proportions and compacted in the shape and size desired. The powders and tablets preferably contain from 5% to 70% or 10% to 70% of the compounds of the present invention.

Suitable solid excipients include, but are not limited to, magnesium carbonate; magnesium stearate; talc; pectin; dextrin; starch; tragacanth; a low melting wax; cocoa butter; carbohydrates; sugars including, but not limited to, lactose, sucrose, mannitol, or sorbitol, starch from corn, wheat, rice, potato, or other plants; cellulose such as methyl cellulose, hydroxypropylmethylcellulose, or sodium carboxymethylcellulose; and gums including arabic and tragacanth; as well as proteins including, but not limited to, gelatin and collagen.

If desired, disintegrating or solubilizing agents may be added, such as the cross-linked polyvinyl pyrrolidone, agar, alginic acid, or a salt thereof, such as sodium alginate.

For preparing suppositories, a low melting wax, such as a mixture of fatty acid glycerides or cocoa butter, is first melted and the compounds of the present invention are dispersed homogeneously therein, as by stirring. The molten homogeneous mixture is then poured into convenient sized molds, allowed to cool, and thereby to solidify.

Liquid form preparations include solutions, suspensions, and emulsions, for example, water or water/propylene glycol solutions. For parenteral injection, liquid preparations can be formulated in solution in aqueous polyethylene glycol solution.

Aqueous solutions suitable for oral use can be prepared by dissolving the compounds of the present invention in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolarity.

Also included are solid form preparations, which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

Oil suspensions can be formulated by suspending the compound of the present invention in a vegetable oil, such as arachis oil, olive oil, sesame oil or coconut oil, or in a mineral oil such as liquid paraffin; or a mixture of these. The oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto, J. Pharmacol. Exp. Ther. 281:93-102, 1997. The pharmaceutical formulations of the invention can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent.

The compositions of the present invention can also be delivered as microspheres for slow release in the body. For example, microspheres can be formulated for administration via intradermal injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). Both transdermal and intradermal routes afford constant delivery for weeks or months.

In some embodiments, the pharmaceutical compositions of the present invention can be formulated for parenteral administration, such as intravenous (IV) administration or administration into a body cavity or lumen of an organ. The formulations for administration will commonly comprise a solution of the compositions of the present invention dissolved in a pharmaceutically acceptable carrier. Among the acceptable vehicles and solvents that can be employed are water and Ringer's solution, an isotonic sodium chloride. In addition, sterile fixed oils can conventionally be employed as a solvent or suspending medium. For this purpose, any bland fixed oil can be employed including synthetic mono- or diglycerides. In addition, fatty acids such as oleic acid can likewise be used in the preparation of injectables. These solutions are sterile and generally free of undesirable matter. These formulations may be sterilized by conventional, well known sterilization techniques. The formulations may contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like. The concentration of the compositions of the present invention in these formulations can vary widely, and will be selected primarily based on fluid volumes, viscosities, body weight, and the like, in accordance with the particular mode of administration selected and the patient's needs. For IV administration, the formulation can be a sterile injectable preparation, such as a sterile injectable aqueous or oleaginous suspension. This suspension can be formulated according to the known art using those suitable dispersing or wetting agents and suspending agents. The sterile injectable preparation can also be a sterile injectable solution or suspension in a nontoxic parenterally-acceptable diluent or solvent, such as a solution of 1,3-butanediol.

In some embodiments, the formulations of the compositions of the present invention can be delivered by the use of liposomes which fuse with the cellular membrane or are endocytosed, for example, by employing ligands attached to the liposome, or attached directly to the oligonucleotide, that bind to surface membrane protein receptors of the cell resulting in endocytosis. By using liposomes, particularly where the liposome surface carries ligands specific for target cells, or are otherwise preferentially directed to a specific organ, one can focus the delivery of the compositions of the present invention into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989).

### Administration

The compositions of the present invention can be administered by any suitable means, including oral, parenteral and topical methods. Transdermal administration methods, by a topical route, can be formulated as applicator sticks, solutions, suspensions, emulsions, gels, creams, ointments, pastes, jellies, paints, powders, and aerosols.

The pharmaceutical preparation is preferably in unit dosage form. In such form the preparation is subdivided into unit doses containing appropriate quantities of the compounds of the present invention. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The compound of the present invention can be present in any suitable amount, and can depend on various factors including, but not limited to, weight and age of the subject, state of the disease, and the like as is known to those of ordinary skill in the art. Suitable dosage ranges for the compounds disclosed herein include from about 0.1 mg to about 10,000 mg, or about 1 mg to about 1000 mg, or about 10 mg to about 750 mg, or about 25 mg to about 500 mg, or about 50 mg to about 250 mg. Suitable dosages for the compound of the present invention include about 1 mg, 5, 10, 20, 30, 40, 50, 60, 70, 80, 90, 100, 200, 300, 400, 500, 600, 700, 800, 900 or 1000 mg. Suitable dose ranges for LSD, 1P-LSD, and ALD-52 include from about 1 microgram to about 1 mg, or about 1 microgram to about 400 micrograms, or about 1 microgram to about 800 micrograms, or about 50 micrograms to about 200 micrograms, or about 100 micrograms, or about 150 micrograms, or about 250 micrograms. Suitable dosage ranges for 2-bromo-LSD include from about 10 micrograms to about 1.0 mg, or about 1.8 mg, or about 30 micrograms per kg, or about 0.1 mg to about 50 mg, or about 1 mg to about 20 mg, or about 20 mg to about 30 mg.

The compounds disclosed herein can be administered at any suitable frequency, interval and duration. For example, the compounds can be administered once an hour, or two, three or more times an hour, once a day, or two, three, or more times per day, or once every 2, 3, 4, 5, 6, or 7 days, so as to provide the preferred dosage level. When the compound of the present invention is administered more than once a day, representative intervals include 5, 10, 15, 20, 30, 45 and 60 minutes, as well as 1, 2, 4, 6, 8, 10, 12, 16, 20, and 24 hours. The compound of the present invention can be administered once, twice, or three or more times, for an hour, for 1 to 6 hours, for 1 to 12 hours, for 1 to 24 hours, for 6 to 12 hours, for 12 to 24 hours, for a single day, for 1 to 7 days, for a single week, for 1 to 4 weeks, for a month, for 1 to 12 months, for a year or more, or even indefinitely.

The composition can also contain other compatible therapeutic agents. The compounds described herein can be used in combination with one another, with other active agents known to be useful in modulating a glucocorticoid receptor, or with adjunctive agents that may not be effective alone, but may contribute to the efficacy of the active agent.

The compounds of the present invention can be co-administered with a second active agent. Co-administration includes administering the compound of the present invention and active agent within 0.5, 1, 2, 4, 6, 8, 10, 12, 16, 20, or 24 hours of each other. Co-administration also includes administering the compound of the present invention and active agent simultaneously, approximately simultaneously (e.g., within about 1, 5, 10, 15, 20, or 30 minutes of each other), or sequentially in any order. Moreover, the compound of the present invention and the active agent can each be administered once a day, or two, three, or more times per day so as to provide the preferred dosage level per day.

In some embodiments, co-administration can be accomplished by co-formulation, such as by preparing a single pharmaceutical composition including both the compound of the present invention and a second active agent. In other embodiments, the compound of the present invention and the second active agent can be formulated separately.

The disclosed compounds and the second active agent can be present in the compositions of the present invention in any suitable weight ratio, such as from about 1:100 to about 100:1 (w/w), or about 1:50 to about 50:1, or about 1:25 to about 25:1, or about 1:10 to about 10:1, or about 1:5 to about 5:1 (w/w). The compound of the present invention and the second active agent can be present in any suitable weight ratio, such as about 1:100 (w/w), 1:50, 1:25, 1:10, 1:5, 1:4, 1:3, 1:2, 1:1, 2:1, 3:1, 4:1, 5:1, 10:1, 25:1, 50:1 or 100:1 (w/w). Other dosages and dosage ratios of the compound of the present invention and the active agent are suitable in the compositions and methods disclosed herein.

### Methods of Treatment

The compounds of the present invention, such as a compound of Formulas (A), (I), (II), (IIa), (IIb), (IIc), and Table 1, can be used for increasing neuronal plasticity. The compounds of the present invention can also be used to treat any brain disease. The compounds of the present invention can also be used for increasing at least one of translation, transcription or secretion of neurotrophic factors.

In some embodiments, a compound of the present invention, such as a compound of Formulas (A), (I), (II), (IIa), (IIb), (IIc), and Table 1, is used to treat neurological diseases. In some embodiments, the compounds have, for example, anti-addictive properties, antidepressant properties, anxiolytic properties, or a combination thereof. In some embodiments, the neurological disease is a neuropsychiatric disease. In some embodiments, the neuropsychiatric disease is a mood or anxiety disorder. In some embodiments, the neurological disease is a migraine, headaches (e.g., cluster headache), post-traumatic stress disorder (PTSD), anxiety, depression, neurodegenerative disorder, Alzheimer's disease, Parkinson's disease, psychological disorder, treatment resistant depression, suicidal ideation, major depressive disorder, bipolar disorder, schizophrenia, stroke, traumatic brain injury, and addiction (e.g., substance use disorder). In some embodiments, the neurological disease is a migraine or cluster headache. In some embodiments, the neurological disease is a neurodegenerative disorder, Alzheimer's disease, or Parkinson's disease. In some embodiments, the neurological disease is a psychological disorder, treatment resistant depression, suicidal ideation, major depressive disorder, bipolar disorder, schizophrenia, post-traumatic stress disorder (PTSD), addiction (e.g., substance use disorder), depression, or anxiety. In some embodiments, the neuropsychiatric disease is a psychological disorder, treatment resistant depression, suicidal ideation, major depressive disorder, bipolar disorder, schizophrenia, post-traumatic stress disorder (PTSD), addiction (e.g., substance use disorder), depression, or anxiety. In some embodiments, the neuropsychiatric disease or neurological disease is post-traumatic stress disorder (PTSD), addiction (e.g., substance use disorder), schizophrenia, depression, or anxiety. In some embodiments, the neuropsychiatric disease or neurological disease is addiction (e.g., substance use disorder). In some embodiments, the neuropsychiatric disease or neurological disease is depression. In some embodiments, the neuropsychiatric disease or neurological disease is anxiety. In some embodiments, the neuropsychiatric disease or neurological disease is post-traumatic stress disorder (PTSD). In some embodiments, the neurological disease is stroke or traumatic brain injury. In some embodiments, the neuropsychiatric disease or neurological disease is schizophrenia.

In some embodiments, a compound of the present invention is used for increasing neuronal plasticity. In some embodiments, the compounds described herein are used for treating a brain disorder. In some embodiments, the compounds described herein are used for increasing at least one of translation, transcription, or secretion of neurotrophic factors.

In some embodiments, the present invention provides a method of treating a disease, including administering to a subject in need thereof, a therapeutically effective amount of a compound of the present invention, such as a compound of Formulas (A), (I), (II), (IIa), (IIb), (IIc), and Table 1. In some embodiments, the disease is a musculoskeletal pain disorder including fibromyalgia, muscle pain, joint stiffness, osteoarthritis, rheumatoid arthritis, muscle cramps. In some embodiments, the present invention provides a method of treating a disease of women's reproductive health including premenstrual dysphoric disorder (PMDD), premenstrual syndrome (PMS), post-partum depression, and menopause.

In some embodiments, the compounds of the present invention, such as a compound of Formulas (A), (I), (II), (IIa), (IIb), (IIc), and Table 1, have activity as 5-HT_{2A} modulators. In some embodiments, the compounds of the present invention elicit a biological response by activating the 5-HT_{2A} receptor (e.g., allosteric modulation or modulation of a biological target that activates the 5-HT_{2A} receptor). 5-HT_{2A} agonism has been correlated with the promotion of neural plasticity (Ly et al., 2018). 5-HT_{2A} antagonists abrogate the neuritogenesis and spinogenesis effects of hallucinogenic compounds with 5-HT_{2A} agonist activity, for example, DMT, LSD, and DOI. In some embodiments, the compounds of the present invention are 5-HT_{2A} modulators and promote neural plasticity (e.g., cortical structural plasticity). In some embodiments, the compounds of the present invention are selective 5-HT_{2A} modulators and promote neural plasticity (e.g., cortical structural plasticity). In some embodiments, promotion of neural plasticity includes, for example, increased dendritic spine growth, increased synthesis of synaptic proteins, strengthened synaptic responses, increased dendritic arbor complexity, increased dendritic branch content, increased spinogenesis, increased neuritogenesis, or any combination thereof. In some embodiments, increased neural plasticity includes, for example, increased cortical structural plasticity in the anterior parts of the brain.

In some embodiments, the 5-HT_{2A} modulators (e.g., 5-HT_{2A} agonists) are non-hallucinogenic. In some embodiments, non-hallucinogenic 5-HT_{2A} modulators (e.g., 5-HT_{2A} agonists) are used to treat neurological diseases, which modulators do not elicit dissociative side-effects. In some embodiments, the hallucinogenic potential of the compounds described herein is assessed in vitro. In some embodiments, the hallucinogenic potential assessed in vitro of the compounds described herein is compared to the hallucinogenic potential assessed in vitro of hallucinogenic homologs. In some embodiments, the compounds described herein elicit less hallucinogenic potential in vitro than the hallucinogenic homologs.

In some embodiments, serotonin receptor modulators, such as modulators of serotonin receptor 2A (5-HT_{2A} modulators, e.g., 5-HT_{2A} agonists), are used to treat a brain disorder. The presently disclosed compounds of Formulas (A), (I), (II), (IIa), (IIb), (IIc), and Table 1 can function as 5-HT_{2A} agonists alone, or in combination with a second therapeutic agent that also is a 5-HT_{2A} modulator. In such cases the second therapeutic agent can be an agonist or an antagonist. In some instances, it may be helpful administer a 5-HT_{2A} antagonist in combination with a compound of the present invention to mitigate undesirable effects of 5-HT_{2A} agonism, such as potential hallucinogenic effects. Serotonin receptor modulators useful as second therapeutic agents for combination therapy as described herein are known to those of skill in the art and include, without limitation, ketanserin, volinanserin (MDL-100907), eplivanserin (SR-46349), pimavanserin (ACP-103), glemanserin (MDL-11939), ritanserin, flibanserin, nelotanserin, blonanserin, mianserin, mirtazapine, roluperiodone (CYR-101, MIN-101), quetiapine, olanzapine, altanserin, acepromazine, nefazodone, risperidone, pruvanserin, AC-90179, AC-279, adatanserin, fananserin, HY10275, benanserin, butanserin, manserin, iferanserin, lidanserin, pelanserin, seganserin, tropanserin, lorcaserin, ICI-169369, methiothepin, methysergide, trazodone, cinitapride, cyproheptadine, brexpiprazole, cariprazine, agomelatine, setoperone, 1-(1-Naphthyl)piperazine, LY-367265, pirenperone, metergoline, deramciclane, amperozide, cinanserin, LY-86057, GSK-215083, cyamemazine, mesulergine, BF-1, LY-215840, sergolexole, spiramide, LY-53857, amesergide, LY-108742, pipamperone, LY-314228, 5-I-R91150, 5-MeO-NBpBrT, 9-Aminomethyl-9,10-dihydroanthracene, niaprazine, SB-215505, SB-204741, SB-206553, SB-242084, LY-272015, SB-243213, SB-200646, RS-102221, zotepine, clozapine, chlorpromazine, sertindole, iloperidone, paliperidone, asenapine, amisulpride, aripiprazole, lurasidone, ziprasidone, lumateperone, perospirone, mosapramine, AMDA (9-Aminomethyl-9,10-dihydroanthracene), xanomeline, buspirone, methiothepin, an extended-release form of olanzapine (e.g., ZYPREXA RELPREVV), an extended-release form of quetiapine, an extended-release form of risperidone (e.g., Risperdal Consta), an extended-release form of paliperidone (e.g., Invega Sustenna and Invega Trinza), an extended-release form of fluphenazine decanoate including Prolixin Decanoate, an extended-release form of aripiprazole lauroxil including Aristada, an extended-release form of aripiprazole including Abilify Maintena, 3-(2-(4-(4-Fluorobenzoyl)piperazin-1-yl)ethyl)-5-methyl-5-phenylimidazolidine-2,4-dione, 3-(2-(4-Benzhydrylpiperazin-1-yl)ethyl)-5-methyl-5-phe-nylimidazolidine-2,4-dione, 3-(3-(4-(2-Fluorophenyl)piperazin-1-yl)propyl)-5-me-thyl-5-phenylimidazolidine-2,4-dione, 3-(3-(4-(3-Fluorophenyl)piperazin-1-yl)propyl)-5-me-thyl-5-phenylimidazolidine-2,4-dione, 3-(3-(4-(4-Fluorophenyl)piperazin-1-yl)propyl)-5-me-thyl-5-phenylimidazolidine-2,4-dione, 3-(3-(4-(4-Fluorobenzoyl)piperazin-1-yl)propyl)-5-methyl-5-phenylimidazolidine-2,4-dione, 3-(2-(4-(4-Fluorobenzoyl)piperazin-1-yl)ethyl)-8-phenyl-1,3-diazaspiro[4.5]decane-2,4-dione, 3-(2-(4-Benzhydrylpiperazin-1-yl)ethyl)-8-phenyl-1,3-diazaspiro[4.5]decane-2,4-dione, 3-(3-(4-(2-Fluorophenyl)piperazin-1-yl)propyl)-8-phe-nyl-1,3-diazaspiro[4.5]decane-2,4-dione, 3-(3-(4-(3-Fluorophenyl)piperazin-1-yl)propyl)-8-phe-nyl-1,3-diazaspiro[4.5]decane-2,4-dione, 3-(3-(4-(4-Fluorophenyl)piperazin-1-yl)propyl)-8-phe-nyl-1,3-diazaspiro[4.5]decane-2,4-dione, and 3-(3-(4-(4-Fluorobenzoyl)piperazin-1-yl)propyl)-8-phenyl-1,3-diazaspiro[4.5]decane-2,4-dione, or a pharmaceutically acceptable salt, solvate, metabolite, deuterated analog, derivative, prodrug, or combinations thereof. In some embodiments, the serotonin receptor modulator used as a second therapeutic is pimavanserin or a pharmaceutically acceptable salt, solvate, metabolite, derivative, or prodrug thereof. In some embodiments, the serotonin receptor modulator is administered prior to a compound disclosed herein, such as about three or about one hours prior to administration of a compound according to Formulas (A), (I), (II), (IIa), (IIb), (IIc), and Table 1. In some embodiments, the serotonin receptor modulator is administered at most about one hour prior to the presently disclosed compound. Thus, in some embodiments of combination therapy with the presently disclosed compounds, the second therapeutic agent is a serotonin receptor modulator. In some embodiments the second therapeutic agent serotonin receptor modulator is provided at a dose of from about 10 mg to about 350 mg. In some embodiments, the serotonin receptor modulator is provided at a dose of from about 20 mg to about 200 mg. In some embodiments, the serotonin receptor modulator is provided at a dose of from about 10 mg to about 100 mg. In certain such embodiments, the compound of the present invention is provided at a dose of from about 10 mg to about 100 mg, or from about 20 to about 200 mg, or from about 15 to about 300 mg, and the serotonin receptor modulator is provided at a dose of about 10 mg to about 100 mg.

In some embodiments, non-hallucinogenic 5-HT_{2A} modulators (e.g., 5-HT_{2A} agonists) are used to treat neurological diseases. In some embodiments, the neurological diseases comprise decreased neural plasticity, decreased cortical structural plasticity, decreased 5-HT_{2A} receptor content, decreased dendritic arbor complexity, loss of dendritic spines, decreased dendritic branch content, decreased spinogenesis, decreased neuritogenesis, retraction of neurites, or any combination thereof.

In some embodiments, non-hallucinogenic 5-HT_{2A} modulators (e.g., 5-HT_{2A} agonists) are used for increasing neuronal plasticity. In some embodiments, non-hallucinogenic 5-HT_{2A} modulators (e.g., 5-HT_{2A} agonists) are used for treating a brain disorder. In some embodiments, non-hallucinogenic 5-HT_{2A} modulators (e.g., 5-FIT_{2A} agonists) are used for increasing at least one of translation, transcription, or secretion of neurotrophic factors.

In some embodiments the presently disclosed compounds of Formulas (A), (I), (II), (IIa), (IIb), (IIc), and Table 1 are given to patients in a low dose that is lower than would produce noticeable psychedelic effects but high enough to provide a therapeutic benefit. This dose range is predicted to be between 200 ug (micrograms) and 2 mg.

### Methods for Increasing Neuronal Plasticity

Neuronal plasticity refers to the ability of the brain to change structure and/or function throughout a subject's life. New neurons can be produced and integrated into the central nervous system throughout the subject's life. Increasing neuronal plasticity includes, but is not limited to, promoting neuronal growth, promoting neuritogenesis, promoting synaptogenesis, promoting dendritogenesis, increasing dendritic arbor complexity, increasing dendritic spine density, and increasing excitatory synapsis in the brain. In some embodiments, increasing neuronal plasticity comprises promoting neuronal growth, promoting neuritogenesis, promoting synaptogenesis, promoting dendritogenesis, increasing dendritic arbor complexity, and increasing dendritic spine density.

In some embodiments, increasing neuronal plasticity by treating a subject with a compound of Formulas (A), (I), (II), (IIa), (IIb), (IIc), and Table 1 can treat neurodegenerative disorder, Alzheimer's, Parkinson's disease, psychological disorder, depression, addiction, anxiety, post-traumatic stress disorder, treatment resistant depression, suicidal ideation, major depressive disorder, bipolar disorder, schizophrenia, stroke, traumatic brain injury, or substance use disorder.

In some embodiments, the present invention provides methods for increasing neuronal plasticity, comprising contacting a neuronal cell with a compound of the present invention, such as a compound of Formulas (A), (I), (II), (IIa), (IIb), (IIc), and Table 1. In some embodiments, increasing neuronal plasticity improves a brain disorder described herein.

In some embodiments, a compound of the present invention is used to increase neuronal plasticity. In some embodiments, the compounds used to increase neuronal plasticity have, for example, anti-addictive properties, antidepressant properties, anxiolytic properties, or a combination thereof. In some embodiments, decreased neuronal plasticity is associated with a neuropsychiatric disease. In some embodiments, the neuropsychiatric disease is a mood or anxiety disorder. In some embodiments, the neuropsychiatric disease includes, for example, migraine, cluster headache, post-traumatic stress disorder (PTSD), schizophrenia, anxiety, depression, and addiction (e.g., substance abuse disorder). In some embodiments, brain disorders include, for example, migraines, addiction (e.g., substance use disorder), depression, and anxiety.

In some embodiments, the experiment or assay to determine increased neuronal plasticity of any compound of the present invention is a phenotypic assay, a dendritogenesis assay, a spinogenesis assay, a synaptogenesis assay, a Sholl analysis, a concentration-response experiment, a 5-HT_{2A} agonist assay, a 5-HT_{2A} antagonist assay, a 5-HT_{2A} binding assay, or a 5-HT_{2A} blocking experiment (e.g., ketanserin blocking experiments). In some embodiments, the experiment or assay to determine the hallucinogenic potential of any compound of the present invention is a mouse head-twitch response (HTR) assay.

In some embodiments, the present invention provides a method for increasing neuronal plasticity, comprising contacting a neuronal cell with a compound of Formulas (A), (I), (II), (IIa), (IIb), (IIc), and Table 1.

### Methods of Treating a Brain Disorder

In some embodiments, the present invention provides a method of treating a disease, including administering to a subject in need thereof, a therapeutically effective amount of a compound of the present invention, such as a compound of Formulas (A), (I), (II), (IIa), (IIb), (IIc), and Table 1. In some embodiments, the disease is a musculoskeletal pain disorder including fibromyalgia, muscle pain, joint stiffness, osteoarthritis, rheumatoid arthritis, muscle cramps. In some embodiments, the present invention provides a method of treating a disease of women's reproductive health including premenstrual dysphoric disorder (PMDD), premenstrual syndrome (PMS), post-partum depression, and menopause. In some embodiments, the present invention provides a method of treating a brain disorder, including administering to a subject in need thereof, a therapeutically effective amount of a compound of the present invention. In some embodiments, the present invention provides a method of treating a brain disorder with combination therapy, including administering to a subject in need thereof, a therapeutically effective amount of a compound of the present invention and at least one additional therapeutic agent.

In some embodiments, 5-HT_{2A} modulators (e.g., 5-HT_{2A} agonists) are used to treat a brain disorder. In some embodiments, the brain disorders comprise decreased neural plasticity, decreased cortical structural plasticity, decreased 5-HT_{2A} receptor content, decreased dendritic arbor complexity, loss of dendritic spines, decreased dendritic branch content, decreased spinogenesis, decreased neuritogenesis, retraction of neurites, or any combination thereof.

In some embodiments, a compound of the present invention, such as a compound of Formulas (A), (I), (II), (IIa), (IIb), (IIc), and Table 1, is used to treat brain disorders. In some embodiments, the compounds have, for example, anti-addictive properties, antidepressant properties, anxiolytic properties, or a combination thereof. In some embodiments, the brain disorder is a neuropsychiatric disease. In some embodiments, the neuropsychiatric disease is a mood or anxiety disorder. In some embodiments, brain disorders include, for example, migraine, cluster headache, post-traumatic stress disorder (PTSD), anxiety, depression, panic disorder, suicidality, schizophrenia, and addiction (e.g., substance abuse disorder). In some embodiments, brain disorders include, for example, migraines, addiction (e.g., substance use disorder), depression, and anxiety.

In some embodiments, the present invention provides a method of treating a brain disorder, comprising administering to a subject in need thereof a therapeutically effective amount of a compound disclosed herein, such as a compound of Formulas (A), (I), (II), (IIa), (IIb), (IIc), and Table 1.

In some embodiments, the brain disorder is a neurodegenerative disorder, Alzheimer's, Parkinson's disease, psychological disorder, depression, addiction, anxiety, post-traumatic stress disorder, treatment resistant depression, suicidal ideation, major depressive disorder, bipolar disorder, schizophrenia, stroke, traumatic brain injury, or substance use disorder.

In some embodiments, the brain disorder is a neurodegenerative disorder, Alzheimer's, or Parkinson's disease. In some embodiments, the brain disorder is a psychological disorder, depression, addiction, anxiety, or a post-traumatic stress disorder. In some embodiments, the brain disorder is depression. In some embodiments, the brain disorder is addiction. In some embodiments, the brain disorder is treatment resistant depression, suicidal ideation, major depressive disorder, bipolar disorder, schizophrenia, stroke, traumatic brain injury or substance use disorder. In some embodiments, the brain disorder is treatment resistant depression, suicidal ideation, major depressive disorder, bipolar disorder, schizophrenia, or substance use disorder. In some embodiments, the brain disorder is stroke or traumatic brain injury. In some embodiments, the brain disorder is treatment resistant depression, suicidal ideation, major depressive disorder, bipolar disorder, or substance use disorder. In some embodiments, the brain disorder is schizophrenia. In some embodiments, the brain disorder is alcohol use disorder.

In some embodiments, the method further comprises administering one or more additional therapeutic agent that is lithium, olanzapine (Zyprexa), quetiapine (Seroquel), risperidone (Risperdal), ariprazole (Abilify), ziprasidone (Geodon), clozapine (Clozaril), divalproex sodium (Depakote), lamotrigine (Lamictal), valproic acid (Depakene), carbamazepine (Equetro), topiramate (Topamax), levomilnacipran (Fetzima), duloxetine (Cymbalta, Yentreve), venlafaxine (Effexor), citalopram (Celexa), fluvoxamine (Luvox), escitalopram (Lexapro), fluoxetine (Prozac), paroxetine (Paxil), sertraline (Zoloft), clomipramine (Anafranil), amitriptyline (Elavil), desipramine (Norpramin), imipramine (Tofranil), nortriptyline (Pamelor), phenelzine (Nardil), tranylcypromine (Parnate), diazepam (Valium), alprazolam (Xanax), or clonazepam (Klonopin).

In some embodiments of the method for treating a brain disorder disclosed herein with a compound according to Formulas (A), (I), (II), (IIa), (IIb), (IIc), and Table 1, a second therapeutic agent that is an empathogenic agent is administered. Examples of suitable empathogenic agents for use in combination with a compound according to Formulas (A), (I), (II), (IIa), (IIb), (IIc), and Table 1 are selected from the phenethylamines, such as 3,4-methylenedioxymethamphetamine (MDMA) and analogs thereof. Other suitable empathogenic agents for use in combination with the presently disclosed compounds include, without limitation,
N-Allyl-3,4-methylenedioxy-amphetamine (MDAL)
N-Butyl-3,4-methylenedioxyamphetamine (MDBU)
N-Benzyl-3,4-methylenedioxyamphetamine (MDBZ)
N-Cyclopropylmethyl-3,4-methylenedioxyamphetamine (MDCPM)
N,N-Dimethyl-3,4-methylenedioxyamphetamine (MDDM)
N-Ethyl-3,4-methylenedioxyamphetamine (MDE; MDEA)
N-(2-Hydroxyethyl)-3,4-methylenedioxy amphetamine (MDHOET)
N-Isopropyl-3,4-methylenedioxyamphetamine (MDIP)
N-Methyl-3,4-ethylenedioxyamphetamine (MDMC)
N-Methoxy-3,4-methylenedioxyamphetamine (MDMEO)
N-(2-Methoxyethyl)-3,4-methylenedioxyamphetamine (MDMEOET)
alpha,alpha,N-Trimethyl-3,4-methylenedioxyphenethylamine (MDMP;
3,4-Methylenedioxy-N-methylphentermine)
N-Hydroxy-3,4-methylenedioxyamphetamine (MDOH)
3,4-Methylenedioxyphenethylamine (MDPEA)
alpha,alpha-Dimethyl-3,4-methylenedioxyphenethylamine (MDPH; 3,4-methylenedioxyphentermine)
N-Propargyl-3,4-methylenedioxyamphetamine (MDPL)
Methylenedioxy-2-aminoindane (MDAI)
N-methyl-1,3-benzodioxolylbutanamine (MBDB)
3,4-methylenedioxy-N-methyl-a-ethylphenylethylamine
3,4-Methylenedioxyamphetamine MDA
Methylone (also known as “3,4-methylenedioxy-N-methylcathinone)
Ethylone, also known as 3,4-methylenedioxy-N-ethylcathinone
GHB or Gamma Hydroxybutyrate or sodium oxybate
N-Propyl-3,4-methylenedioxyamphetamine (MDPR), and the like.

In some embodiments, the compounds of the present invention are used in combination with the standard of care therapy for a neurological disease described herein. Non-limiting examples of the standard of care therapies, may include, for example, lithium, olanzapine, quetiapine, risperidone, ariprazole, ziprasidone, clozapine, divalproex sodium, lamotrigine, valproic acid, carbamazepine, topiramate, levomilnacipran, duloxetine, venlafaxine, citalopram, fluvoxamine, escitalopram, fluoxetine, paroxetine, sertraline, clomipramine, amitriptyline, desipramine, imipramine, nortriptyline, phenelzine, tranylcypromine, diazepam, alprazolam, clonazepam, or any combination thereof. Nonlimiting examples of standard of care therapy for depression are sertraline, fluoxetine, escitalopram, venlafaxine, or aripiprazole. Non-limiting examples of standard of care therapy for depression are citralopram, escitalopram, fluoxetine, paroxetine, diazepam, or sertraline. Additional examples of standard of care therapeutics are known to those of ordinary skill in the art.

### Methods of Increasing at Least One of Translation, Transcription, or Secretion of Neurotrophic Factors

Neurotrophic factors refers to a family of soluble peptides or proteins which support the survival, growth, and differentiation of developing and mature neurons. Increasing at least one of translation, transcription, or secretion of neurotrophic factors can be useful for, but not limited to, increasing neuronal plasticity, promoting neuronal growth, promoting neuritogenesis, promoting synaptogenesis, promoting dendritogenesis, increasing dendritic arbor complexity, increasing dendritic spine density, and increasing excitatory synapsis in the brain. In some embodiments, increasing at least one of translation, transcription, or secretion of neurotrophic factors can increasing neuronal plasticity. In some embodiments, increasing at least one of translation, transcription, or secretion of neurotrophic factors can promoting neuronal growth, promoting neuritogenesis, promoting synaptogenesis, promoting dendritogenesis, increasing dendritic arbor complexity, and/or increasing dendritic spine density.

In some embodiments, 5-HT_{2A} modulators (e.g., 5-HT_{2A} agonists) are used to increase at least one of translation, transcription, or secretion of neurotrophic factors. In some embodiments, a compound of the present invention, such as a compound of Formula I, is used to increase at least one of translation, transcription, or secretion of neurotrophic factors. In some embodiments, increasing at least one of translation, transcription or secretion of neurotrophic factors treats a migraine, headaches (e.g., cluster headache), post-traumatic stress disorder (PTSD), anxiety, depression, neurodegenerative disorder, Alzheimer's disease, Parkinson's disease, psychological disorder, treatment resistant depression, suicidal ideation, major depressive disorder, bipolar disorder, schizophrenia, stroke, traumatic brain injury, and addiction (e.g., substance use disorder).

In some embodiments, the experiment or assay used to determine increase translation of neurotrophic factors includes ELISA, western blot, immunofluorescence assays, proteomic experiments, and mass spectrometry. In some embodiments, the experiment or assay used to determine increase transcription of neurotrophic factors includes gene expression assays, PCR, and microarrays. In some embodiments, the experiment or assay used to determine increase secretion of neurotrophic factors includes ELISA, western blot, immunofluorescence assays, proteomic experiments, and mass spectrometry.

In some embodiments, the present invention provides a method for increasing at least one of translation, transcription or secretion of neurotrophic factors, comprising contacting a neuronal cell with a compound disclosed herein, such as a compound of Formulas (A), (I), (II), (IIa), (IIb), (IIc), and Table 1.

### EXAMPLES

Exemplary compounds disclosed herein are prepared from the isotopically enriched building blocks analogous to those used to synthesize the unenriched compounds.

### Example 1: Synthesis of (6aR,9R)-5-Bromo-N,N-diethyl-7-methyl-4,6,6a,7,8,9-hexahydroindolo[4,3-fg]quinoline-9-carboxamide-1,2,3-d₃ formate (compound 1)

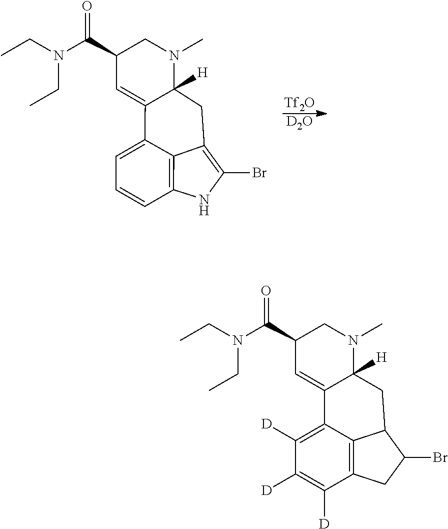 [SMILES: "Tf2O" ; CCN(CC)C(=O)[C@@H]1C=C2c3cccc4[nH]c(Br)c(c34)C[C@H]2N(C)C1 ; "D2O" ; [2H]c1c([2H])c2c3c(c1[2H])C1=C[C@@H](C(=O)N(CC)CC)CN(C)[C@@H]1CC3C(Br)C2]

To trifluoromethanesulfonic anhydride (1.26 g, 0.75 mL, 4.5 mmol, 18 equiv.) was added D₂O (90 mg, 80 μL, 4.5 mmol, 18 equiv.) dropwise at 0° C. in a sealed microwave vessel under an atmosphere of nitrogen. The solution was heated to 80° C. and stirred for 1 h, after which the layers combined. The mixture was cooled to rt and 2-Br-LSD (100 mg, 0.25 mmol, 1 equiv.) was added portion wise, then stirred at rt for 1 h. The mixture was cooled to 0° C., diluted with DCM (20 mL), quenched with satd. aqueous NaHCO₃ (25 mL) and the layers separated. The aqueous phase was extracted with DCM (3×25 mL), the combined organic layers were washed satd. brine (35 mL), dried (MgSO₄), filtered and concentrated to give a solid (120 mg). This material was purified by column chromatography on silica gel, eluting with a gradient of MeOH in DCM then reverse-phase chromatography on C18 silica eluting with a gradient of acetonitrile in H₂O (0.1% formic acid) to obtain (6aR,9R)-5-bromo-N,N-diethyl-7-methyl-4,6,6a,7,8,9-hexahydroindolo[4,3-fg]quinoline-9-carboxamide-1,2,3-d₃ (7.1 mg, 55%) as a solid. m/z=405.15 and 407.15 [M+H]⁺; ¹H NMR (300 MHz, CD₃OD) δ 6.32 (s, 1H, C═CH), 4.02 (m, 1H, CHCO), 3.57 (m, 2H, CHH and CHN), 3.47 (m, 4H, 2×CH₂), 3.21 (m, 1H, CHH), 2.95 (m, 1H, CHH), 2.74 (s, 3H, NCH₃), 2.60 (m, 1H, CHH), 1.32 (t, 3H, J=7.1 Hz, CH₃), 1.32 (t, 3H, J=7.1 Hz, CH₃).

### Example 2: Synthesis of (6aR,9R)-5-bromo-N,N-diethyl-7-(methyl-d₃)-4,6,6a,7,8,9-hexahydroindolo[4,3-fg]quinoline-9-carboxamide (2-Br-LSD-d₃) (compound 2)

### Step 1: Preparation of (6aR,9R)-5-bromo-N,N-diethyl-4,6,6a,7,8,9-hexahydroindolo[4,3-fg]quinoline-9-carboxamide

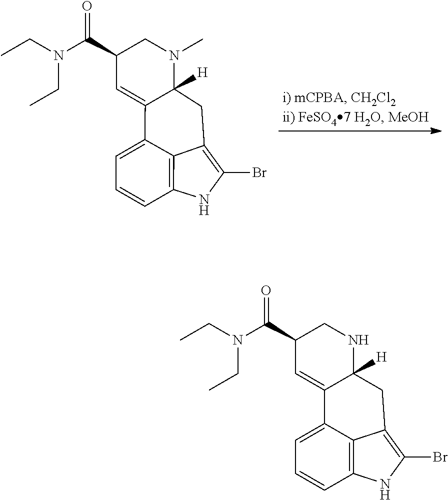 [SMILES: CCN(CC)C(=O)[C@@H]1C=C2c3cccc4[nH]c(Br)c(c34)C[C@H]2N(C)C1 ; CCN(CC)C(=O)[C@@H]1C=C2c3cccc4[nH]c(Br)c(c34)C[C@H]2NC1]

To a solution of 2-Br-LSD (200 mg, 0.50 mmol, 1 equiv.) in DCM (5 mL) at 0° C. was added 3-chloroperbenzoic acid (103 mg, 0.60 mmol, 1.2 equiv.) portion wise. The mixture was stirred at 0° C. for 40 min, then a suspension of iron(II) sulfate heptahydrate (69 mg, 0.25 mmol, 0.5 equiv.) in MeOH (0.3 mL) was added at 0° C. and the mixture was stirred for 1.5 h. The mixture was quenched with saturated aqueous Na₂S₂O₃ (15 mL) and extracted with DCM (3×5 mL). The combined organic layers were washed with saturated aqueous NaHCO₃ (20 mL), dried (Na₂SO₄), filtered and concentrated. The residue was purified by column chromatography on silica gel, eluting with a gradient of MeOH in DCM to afford (6aR,9R)-5-bromo-N,N-diethyl-4,6,6a,7,8,9-hexahydroindolo[4,3-fg]quinoline-9-carboxamide (64 mg) as a semi-solid. This material was used without further purification. m/z=388.10 and 390.10 [M+H]⁺; ¹H NMR (300 MHz, CDCl₃) δ 7.94 (m, 1H, NH), 7.37 (d, 1H, J=7.5 Hz, NH), 7.11 (m, 3H, 3×ArH), 6.33 (s, 1H, C═CH), 3.92 (bs, 1H, CHCO), 3.45 (m, 6H, 2×CH₂Me, CHN and CHH), 3.28 (dd, 1H, J=14.9, 5.9 Hz, CHH), 2.83 (dd, 1H, J=14.8, 11.9 Hz, CHH), 1.21 (m, 6H, 2×CH₃).

### Step 2: Preparation of (6aR,9R)-5-bromo-N,N-diethyl-7-(methyl-d₃)-4,6,6a,7,8,9-hexahydroindolo[4,3-fg]quinoline-9-carboxamide (2-Br-LSD-d₃)

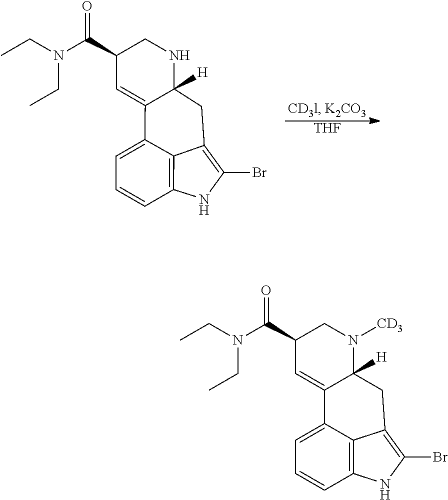 [SMILES: CCN(CC)C(=O)[C@@H]1C=C2c3cccc4[nH]c(Br)c(c34)C[C@H]2NC1 ; "CD3l, K2CO3" ; [2H]C([2H])([2H])N1C[C@H](C(=O)N(CC)CC)C=C2c3cccc4[nH]c(Br)c(c34)C[C@H]21 ; "THF"]

To a solution of (6aR,9R)-5-bromo-N,N-diethyl-4,6,6a,7,8,9-hexahydroindolo[4,3-fg]quinoline-9-carboxamide (64 mg, 0.17 mmol, 1 equiv.) in anhydrous THE (2 mL) was added K₂CO₃ (23 mg, 0.17 mmol, 1 equiv.) and iodomethane-d₃ (26 mg, 11 L, 0.18 mmol, 1.1 equiv.). The mixture was heated to 60° C. and stirred for 3 h, then diluted with H₂O (20 mL) and extracted with DCM (3×5 mL). The combined organic layers were washed with satd. brine (20 mL), dried (Na₂SO₄), filtered and concentrated. The residue was purified by column chromatography on silica gel, eluting with a gradient of MeOH in DCM, to give (6aR,9R)-5-bromo-N,N-diethyl-7-(methyl-d₃)-4,6,6a,7,8,9-hexahydroindolo[4,3-fg]quinoline-9-carboxamide (12 mg, 18%) as a solid. m/z=405.15 and 407.15 [M+H]⁺; ¹H NMR (300 MHz, CDCl₃) δ 7.90 (s, 1H, NH), 7.21 (m, 1H, ArH), 7.13 (m, 2H, ArH), 6.35 (s, 1H, C═CH), 3.88 (m, 1H, CHCO), 3.42 (m, 5H, 2×CH₂Me and CHN), 3.20 (m, 1H, CHH), 3.05 (m, 1H, CHH), 2.88 (t, 1H, J=10.9 Hz, CHH), 2.53 (dd, 1H, J=14.8, 11.3 Hz, CHH), 1.25 (t, 3H, J=7.1 Hz, CH₃), 1.17 (t, 3H, J=7.1 Hz, CH₃).

### Example 3: Synthesis of (6aR,9R)-5-bromo-N,N-bis(ethyl-d₅)-7-methyl-4,6,6a,7,8,9-hexahydroindolo[4,3-fg]quinoline-9-carboxamide (Compound 7)

### Step 1: Preparation of (6aR,9R)—N,N-bis(ethyl-d₅)-7-methyl-4,6,6a,7,8,9-hexahydroindolo[4,3-fg]quinoline-9-carboxamide

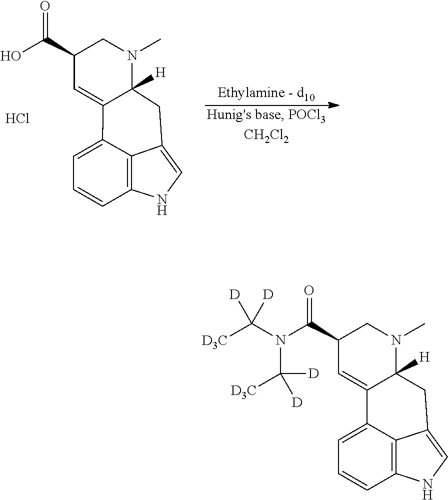 [SMILES: Cl ; CN1C[C@H](C(=O)O)C=C2c3cccc4[nH]cc(c34)C[C@H]21 ; "Ethylamine - d10" ; "Hunig's base, POCl3CH2Cl2" ; [2H]C([2H])([2H])C([2H])([2H])N(C(=O)[C@@H]1C=C2c3cccc4[nH]cc(c34)C[C@H]2N(C)C1)C([2H])([2H])C([2H])([2H])[2H]]

To a suspension of lysergic acid hydrochloride (150 mg, 0.49 mmol, 1 equiv.) in DCM (5 mL) was added Hünig's base (320 mg, 430 μL, 2.47 mmol, 5 equiv.) and the mixture was stirred at rt for 10 min. d₁₀-Diethylamine (44 mg, 55 μL, 0.53 mmol, 1.1 equiv.) and phosphorus oxychloride (150 mg, 92 μL, 0.53 mmol, 1.1 equiv.) were added dropwise and the mixture was stirred at rt for 1 h. The solution was diluted with DCM (20 mL) and washed with satd. aqueous NaHCO₃ solution (2×25 mL), dried (MgSO₄), filtered and concentrated. This material was purified by column chromatography on silica gel, eluting with a gradient of MeOH in DCM to afford (6aR,9R)—N,N-bis(ethyl-d₅)-7-methyl-4,6,6a,7,8,9-hexahydroindolo[4,3-fg]quinoline-9-carboxamide (108 mg, 66%) as a semi-solid. m/z=334.30 [M+H]*; ¹H NMR (300 MHz, CD₃OD) δ 7.16 (m, 3H, 3×ArH), 6.98 (d, 1H, J=1.5 Hz, ArH), 6.32 (s, 1H, C═CH), 3.99 (m, 1H, CHCO), 3.62 (dd, 1H, J=14.4, 5.7 Hz, CHN), 3.22 (m, 1H, CHH), 3.11 (m, 1H, CHH), 2.79 (t, 1H, J=10.9 Hz, CHH), 2.72 (m, 1H, CHH), 2.63 (s, 3H, NCH₃).

### Step 2: Preparation of (6aR,9R)-5-bromo-N,N-bis(ethyl-d₅)-7-methyl-4,6,6a,7,8,9-hexahydroindolo[4,3-fg]quinoline-9-carboxamide

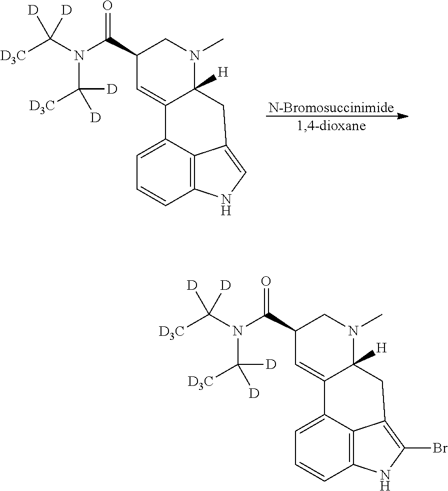 [SMILES: [2H]C([2H])([2H])C([2H])([2H])N(C(=O)[C@@H]1C=C2c3cccc4[nH]cc(c34)C[C@H]2N(C)C1)C([2H])([2H])C([2H])([2H])[2H] ; "1,4-dioxane" ; "N-Bromosuccinimide" ; [2H]C([2H])([2H])C([2H])([2H])N(C(=O)[C@@H]1C=C2c3cccc4[nH]c(Br)c(c34)C[C@H]2N(C)C1)C([2H])([2H])C([2H])([2H])[2H]]

A solution of d₁₀-LSD (250 mg, 0.75 mmol, 1 equiv.) in anhydrous 1,4-dioxane (5.5 mL) was heated to 65° C. and a solution of N-bromosuccinimide (160 mg, 0.90 mmol, 1.2 equiv.) in anhydrous 1,4-dioxane (1.9 mL) was added dropwise. The mixture was stirred at 65° C. for 2 h and then concentrated under vacuum. The residue was redissolved in DCM (35 mL), washed with satd. aqueous NaHCO₃ (2×30 mL) and the layers were separated. The combined aqueous washes were extracted with DCM (3×30 mL) and the combined organic layers were dried (NaSO₄), filtered and concentrated. This material was purified by column chromatography on silica gel, eluting with a gradient of MeOH in DCM to afford (6aR,9R)-5-bromo-N,N-bis(ethyl-d₅)-7-methyl-4,6,6a,7,8,9-hexahydroindolo[4,3-fg]quinoline-9-carboxamide (218 mg, 70%) as a solid. m/z=412.15 and 414.15 [M+H]*; ¹H NMR (300 MHz, CD₃OD) δ 7.14 (m, 3H, 3×ArH), 6.34 (s, 1H, C═CH), 3.99 (m, 1H, CHCO), 3.44 (dd, 1H, J=14.5, 5.6 Hz, CHN), 3.22 (m, 1H, CHH), 3.11 (ddd, 1H, J=11.2, 5.0, 1.2 Hz, CHH), 2.78 (t, 1H, J=11.2 Hz, CHH), 2.63 (s, 3H, NCH₃), 2.53 (d, 1H, J=14.6 Hz, CHH); ¹³C NMR (75.5 MHz, CD₃OD) δ 172.4, 135.4, 134.6, 126.1, 126.1, 122.7, 119.1, 112.2, 109.0, 108.9, 103.4, 62.7, 55.5, 42.5, 39.2, 31.7, 29.2, 26.1.

### Example 4: Synthesis of (6aR,9R)-5-bromo-N,N-bis(ethyl-d₅)-7-(methyl-d₃)-4,6,6a,7,8,9-hexahydroindolo[4,3-fg]quinoline-9-carboxamide trifluoroacetate-d (Compound 9)

### Step 1: Preparation of tert-butyl (6aR,9R)-9-(Bis(ethyl-d₅)carbamoyl)-5-bromo-7-methyl-6a,7,8,9-tetrahydroindolo[4,3-fg]quinoline-4(6H)-carboxylate

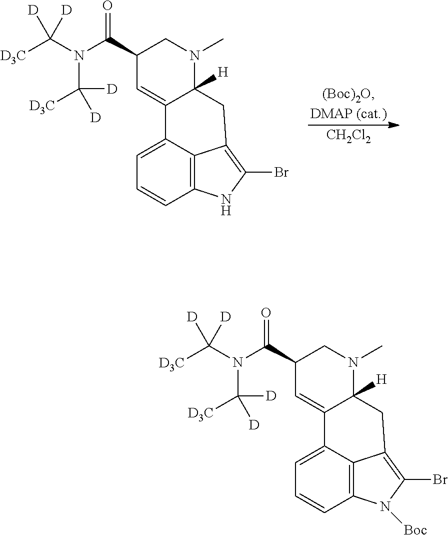 [SMILES: [2H]C([2H])([2H])C([2H])([2H])N(C(=O)[C@@H]1C=C2c3cccc4[nH]c(Br)c(c34)C[C@H]2N(C)C1)C([2H])([2H])C([2H])([2H])[2H] ; "CH2Cl2" ; "(Boc)2O,DMAP (cat.)" ; [2H]C([2H])([2H])C([2H])([2H])N(C(=O)[C@@H]1C=C2c3cccc4c3c(c(Br)n4C(=O)OC(C)(C)C)C[C@H]2N(C)C1)C([2H])([2H])C([2H])([2H])[2H]]

A mixture of d₁₀-2-Br-LSD (192 mg, 0.47 mmol, 1 equiv.), di-tert-butyl carbonate (111 mg, 0.51 mmol, 1.1 equiv.) and DMAP (6 mg, 0.05 mmol, 0.1 equiv.) in DCM (5 mL) was stirred at rt for 3 h. The mixture was diluted with DCM (20 mL), washed with H₂O (25 mL) and the layers separated. The aqueous phase was extracted with DCM (3×25 mL) and the combined organic layers were washed with satd. brine, dried (MgSO₄), filtered and concentrated to give a semi-solid. This material was purified by column chromatography on silica gel, eluting with a gradient of MeOH in DCM to afford tert-butyl (6aR,9R)-9-(bis(ethyl-d₅)carbamoyl)-5-bromo-7-methyl-6a,7,8,9-tetrahydroindolo[4,3-fg]quinoline-4(6H)-carboxylate (225 mg, 93%) as a foam. m/z=512.25 and 514.25 [M+H]⁺; ¹H NMR (300 MHz, CD₃OD) δ 7.79 (d, 1H, J=8.09 Hz, ArH), 7.37 (d, 1H, J=7.52 Hz, ArH), 7.27 (m, 1H, ArH) 6.38 (s, 1H, C═CH), 3.94 (m, 1H, CHCO), 3.42 (dd, 1H, J=15.4, 5.7 Hz, CHN), 3.19 (m, 1H, CHH), 3.09 (ddd, 1H, J=11.4, 5.2, 1.4 Hz, CHH), 2.72 (m, 1H, CHH), 2.60 (s, 3H, NCH₃), 2.42 (dd, 1H, J=15.3, 11.6 Hz, CHH), 1.69 (s, 9H, 3×CH₃).

### Step 2: Preparation of tert-butyl (6aR,9R)-9-(bis(ethyl-d₅)carbamoyl)-5-bromo-6a,7,8,9-tetrahydroindolo[4,3-fg]quinoline-4(6H)-carboxylate

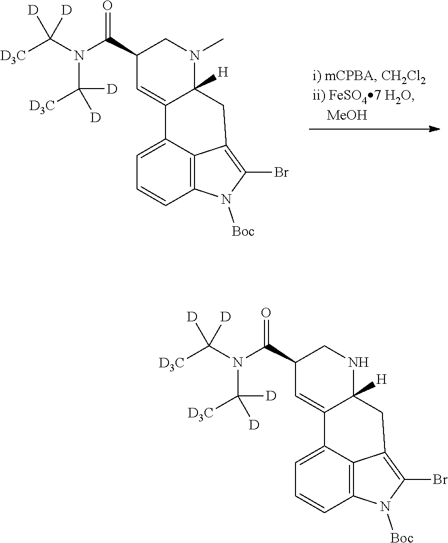 [SMILES: [2H]C([2H])([2H])C([2H])([2H])N(C(=O)[C@@H]1C=C2c3cccc4c3c(c(Br)n4C(=O)OC(C)(C)C)C[C@H]2N(C)C1)C([2H])([2H])C([2H])([2H])[2H] ; [2H]C([2H])([2H])C([2H])([2H])N(C(=O)[C@@H]1C=C2c3cccc4c3c(c(Br)n4C(=O)OC(C)(C)C)C[C@H]2NC1)C([2H])([2H])C([2H])([2H])[2H]]

To a solution of tert-butyl (6aR,9R)-9-(bis(ethyl-d₅)carbamoyl)-5-bromo-7-methyl-6a,7,8,9-tetrahydroindolo[4,3-fg]quinoline-4(6H)-carboxylate (131 mg, 0.26 mmol, 1 equiv.) in DCM (5 mL) at 0° C. was added 3-chloroperbenzoic acid (53 mg, 0.31 mmol, 1.2 equiv.) portion wise. The mixture was stirred at 0° C. for 1 h, then a suspension of iron(II) sulfate heptahydrate (36 mg, 0.13 mmol, 0.5 equiv.) in MeOH (1 mL) was added at 0° C. and the mixture was warmed to rt and stirred for 4 h. The mixture was diluted with saturated aqueous sodium thiosulfate (20 mL), extracted with DCM (3×5 mL) and the combined organic layers were washed with saturated sodium bicarbonate (20 mL), dried (Na₂SO₄), filtered and concentrated. The residue was purified by column chromatography on silica gel, eluting with a gradient of MeOH in DCM, to afford tert-butyl (6aR,9R)-9-(bis(ethyl-d₅)carbamoyl)-5-bromo-6a,7,8,9-tetrahydroindolo[4,3-fg]quinoline-4(6H)-carboxylate (25 mg, 20%) as a semi-solid. m/z=500.20 and 502.20 [M+H]*; ¹H NMR (300 MHz, CDCl₃) δ 7.80 (m, 1H, ArH), 7.30 (m, 2H, obscured by solvent resonance, ArH), 6.37 (s, 1H, C═CH), 3.96 (s, 1H, CHCO), 3.75 (s, 1H, CHN), 3.36 (d, 2H, J=6.8 Hz, CH₂), 3.20 (dd, 1H, J=15.4, 5.8 Hz, CHH), 2.69 (m, 1H, CHH), 1.69 (s, 9H, 3×CH₃).

### Step 3: Preparation of tert-butyl (6aR,9R)-9-(bis(ethyl-d₅)carbamoyl)-5-bromo-7-(methyl-d₃)-6a,7,8,9-tetrahydroindolo[4,3-fg]quinoline-4(6H)-carboxylate

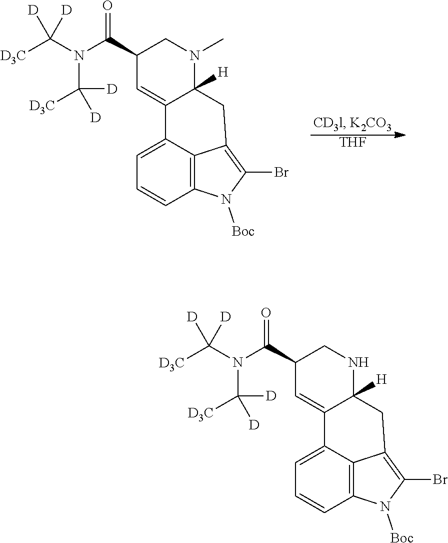 [SMILES: [2H]C([2H])([2H])C([2H])([2H])N(C(=O)[C@@H]1C=C2c3cccc4c3c(c(Br)n4C(=O)OC(C)(C)C)C[C@H]2N(C)C1)C([2H])([2H])C([2H])([2H])[2H] ; "CD3l, K2CO3" ; [2H]C([2H])([2H])C([2H])([2H])N(C(=O)[C@@H]1C=C2c3cccc4c3c(c(Br)n4C(=O)OC(C)(C)C)C[C@H]2NC1)C([2H])([2H])C([2H])([2H])[2H] ; "THF"]

To a solution of tert-butyl (6aR,9R)-9-(bis(ethyl-d₅)carbamoyl)-5-bromo-6a,7,8,9-tetrahydroindolo[4,3-fg]quinoline-4(6H)-carboxylate (21 mg, 0.042 mmol, 1 equiv.) in anhydrous THE (1 mL) was added K₂CO₃ (6 mg, 0.042 mmol, 1 equiv.) and iodomethane-d₃ (7 mg, 3 μL, 0.046 mmol, 1.1 equiv.). The mixture was heated to 60° C. and stirred for 3 h, then diluted with H₂O (5 mL) and extracted with DCM (3×5 mL). The combined organic layers were washed with satd. Brine (5 mL), dried (Na₂SO₄), filtered and concentrated to give a semi-solid residue (20 mg, 92%). m/z=515.25 and 517.25 [M+H]⁺; ¹H NMR (300 MHz, CDCl₃) δ 7.77 (d, 1H, J=8.0 Hz, ArH), 7.34 (d, 1H, J=17.4 Hz, ArH), 7.23 (m, 1H, obscured by solvent resonance, ArH), 6.36 (s, 1H, C═CH), 3.88 (m, 1H, CHCO), 3.37 (dd, 1H, J=15.3, 5.6 Hz, CHN), 3.18 (m, 1H, CHH), 3.05 (m, 1H, CHH), 2.89 (m, 1H, CHH), 2.48 (m, 1H, CHH), 1.69 (s, 9H, 3×CH₃).

### Step 4: Preparation of (6aR,9R)-5-bromo-N,N-bis(ethyl-d₅)-7-(methyl-d₃)-4,6,6a,7,8,9-hexahydroindolo[4,3-fg]quinoline-9-carboxamide trifluoroacetate-d

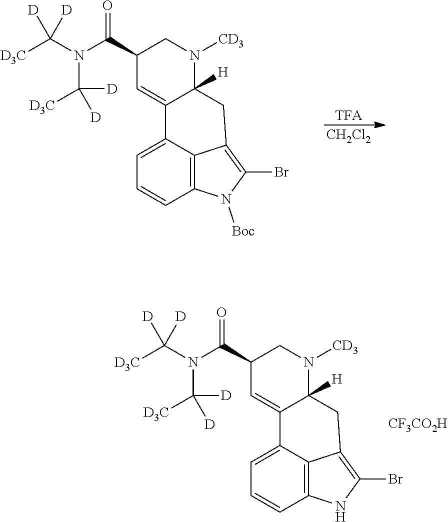 [SMILES: [2H]C([2H])([2H])N1C[C@H](C(=O)N(C([2H])([2H])C([2H])([2H])[2H])C([2H])([2H])C([2H])([2H])[2H])C=C2c3cccc4c3c(c(Br)n4C(=O)OC(C)(C)C)C[C@H]21 ; "CH2Cl2" ; [2H]C([2H])([2H])N1C[C@H](C(=O)N(C([2H])([2H])C([2H])([2H])[2H])C([2H])([2H])C([2H])([2H])[2H])C=C2c3cccc4[nH]c(Br)c(c34)C[C@H]21 ; "CF3CO2H" ; "TFA"]

A solution of tert-butyl (6aR,9R)-9-(bis(ethyl-d₅)carbamoyl)-5-bromo-7-(methyl-d₃)-6a,7,8,9-tetrahydroindolo[4,3-fg]quinoline-4(6H)-carboxylate (20 mg, 0.039 mmol, 1 equiv.) in in a 1:1 mixture of TFA-d:DCM (1.0 mL) was stirred at rt for 1 h. The mixture was concentrated and the residue azeotroped with chloroform (3 mL) to give 14 mg of a semi-solid. The residue was purified by column chromatography on silica gel, eluting with a gradient of MeOH in DCM, to give (6aR,9R)-5-bromo-N,N-bis(ethyl-d₅)-7-(methyl-d₃)-4,6,6a,7,8,9-hexahydroindolo[4,3-fg]quinoline-9-carboxamide trifluoroacetate-d (4 mg, 19%) as a powder. m/z=415.20 and 417.20 [M+H]⁺; ¹H NMR (300 MHz, CD₃OD) δ 7.13 (m, 3H, ArH), 6.38 (s, 1H, C═CH), 4.02 (m, 1H, CHCO), 3.58 (m, 1H, CHN), 3.47 (m, 1H, CHH), 3.27 (m, 1H, obscured by solvent resonance, CHH), 3.05 (t, 1H, J=10.8 Hz, CHH), 2.64 (dd, 1H, J=14.4, 11.4 Hz, CHH).

### Example 5: Synthesis of (6aR,9R)—N,N-Bis(ethyl-d₅)-7-methyl-4,6,6a,7,8,9-hexahydroindolo[4,3-fg]quinoline-9-carboxamide (Compound 47)

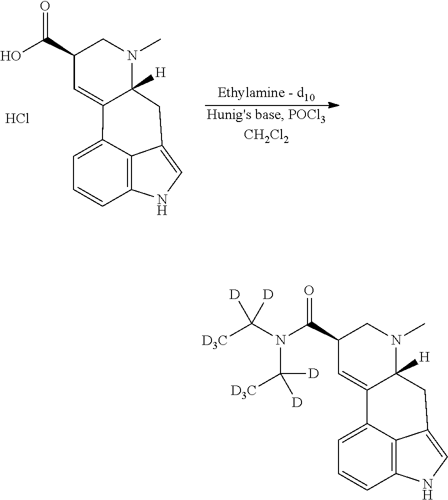 [SMILES: Cl ; CN1C[C@H](C(=O)O)C=C2c3cccc4[nH]cc(c34)C[C@H]21 ; "Ethylamine - d10" ; "Hunig's base, POCl3CH2Cl2" ; [2H]C([2H])([2H])C([2H])([2H])N(C(=O)[C@@H]1C=C2c3cccc4[nH]cc(c34)C[C@H]2N(C)C1)C([2H])([2H])C([2H])([2H])[2H]]

To a suspension of lysergic acid hydrochloride (150 mg, 0.49 mmol, 1 equiv.) in DCM (5 mL) was added Hunig's base (320 mg, 430 μL, 2.47 mmol, 5 equiv.) and the mixture was stirred at rt for 10 min. d₁₀-Diethylamine (44 mg, 55 μL, 0.53 mmol, 1.1 equiv.) and phosphorus oxychloride (150 mg, 92 μL, 0.53 mmol, 1.1 equiv.) were added dropwise and the mixture was stirred at rt for 1 h, then diluted with DCM (20 mL) and washed with satd. Aqueous NaHCO₃ solution (2×25 mL). The organic layer was dried (MgSO₄), filtered and concentrated to give a residue. This material was purified by column chromatography on silica gel, eluting with a gradient of MeOH in DCM to afford (6aR,9R)—N,N-bis(ethyl-d₅)-7-methyl-4,6,6a,7,8,9-hexahydroindolo[4,3-fg]quinoline-9-carboxamide (108 mg, 66%) as a semi-solid. m/z=334.30 [M+H]⁺; ¹H NMR (300 MHz, CD₃OD) δ 7.16 (m, 3H, 3×ArH), 6.98 (d, 1H, J=1.5 Hz, ArH), 6.32 (s, 1H, C═CH), 3.99 (m, 1H, CHCO), 3.62 (dd, 1H, J=14.4, 5.7 Hz, CHN), 3.22 (m, 1H, CHH), 3.11 (m, 1H, CHH), 2.79 (t, 1H, J=10.9 Hz, CHH), 2.72 (m, 1H, CHH), 2.63 (s, 3H, NCH₃).

### Example 6: Synthesis of (6aR,9R)—N,N-Bis(ethyl-d₅)-7-methyl-4,6,6a,7,8,9-hexahydroindolo[4,3-fg]quinoline-9-carboxamide-1,2,3,5-d₄ triflate-d (Compound 67)

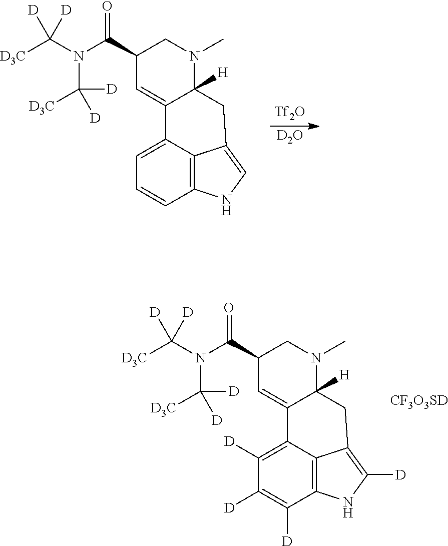

To trifluoromethanesulfonic anhydride (450 mg, 0.27 mL, 1.60 mmol, 18 equiv.) was added D₂O (30 mg, 36 μL, 1.60 mmol, 18 equiv.) dropwise at 0° C. in a sealed microwave vessel under an atmosphere of N₂. The solution was heated to 80° C. and stirred for 1 h, after which the layers combined. The mixture was cooled to rt and d₁₀-LSD (30 mg, 0.09 mmol, 1 equiv.) was added portion-wise and the mixture was stirred at rt for 1 h. The mixture was cooled to 0° C., diluted with DCM (20 mL), quenched with satd. aqueous NaHCO₃ (25 mL) and the layers separated. The aqueous phase was extracted with DCM (3×25 mL), and the combined organic layers were washed with satd. brine (35 mL), dried (MgSO₄), filtered and concentrated to obtain an oil. This material was purified by column chromatography on silica gel, eluting with a gradient of MeOH in DCM to afford (6aR,9R)—N,N-bis(ethyl-d₅)-7-methyl-4,6,6a,7,8,9-hexahydroindolo[4,3-fg]quinoline-9-carboxamide-1,2,3,5-d₄ (18 mg, 59%) as a solid. m/z=338.20 [M+H]⁺; ¹H NMR (300 MHz, CD₃OD) δ 7.90 (s, 1H, NH), 7.16 (m, 0.6H, ArH), 6.32 (s, 0.18H, ArH), 6.35 (s, 1H, C═CH), 3.88 (m, 1H, CHCO), 3.56 (dd, 1H, J=14.3, 5.3 Hz, CHN), 3.24 (m, 1H, CHH), 3.11 (ddd, 1H, J=11.2, 4.9, 1.2 Hz, CHH), 2.90 (t, 1H, J=10.9 Hz, CHH), 2.68 (dd, 1H, J=14.5, 11.4 Hz, CHH), 2.60 (s, 3H, NCH₃).

### Example 7: Synthesis of (6aR,9R)—N,N-Bis(ethyl-d₅)-7-methyl-4,6,6a,7,8,9-hexahydroindolo[4,3-fg]quinoline-9-carboxamide-5-d trifluoroacetate-d (Compound 55)

### Step 1: Preparation of tert-Butyl (6aR,9R)-9-(bis(ethyl-d₅)carbamoyl)-7-methyl-6a,7,8,9-tetrahydroindolo[4,3-fg]quinoline-4(6H)-carboxylate-5-d

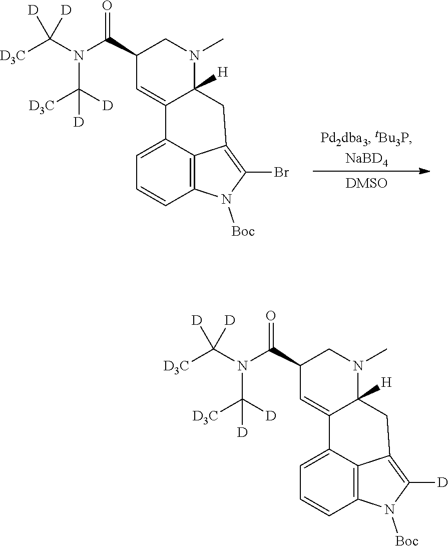 [SMILES: [2H]C([2H])([2H])C([2H])([2H])N(C(=O)[C@@H]1C=C2c3cccc4c3c(c(Br)n4C(=O)OC(C)(C)C)C[C@H]2N(C)C1)C([2H])([2H])C([2H])([2H])[2H] ; [2H]c1c2c3c(cccc3n1C(=O)OC(C)(C)C)C1=C[C@@H](C(=O)N(C([2H])([2H])C([2H])([2H])[2H])C([2H])([2H])C([2H])([2H])[2H])CN(C)[C@@H]1C2]

d₁₀-2-Br—N-Boc-LSD (70 mg, 0.14 mmol, 1 equiv.), Pd₂(dba)₃ (2.8 mg, 0.002 mmol, 0.02 equiv.), NaBD₄ (11 mg, 0.27 mmol, 2 equiv.) and ^{t}Bu₃P (1.6 mg, 0.008 mmol, 0.06 equiv.) were dissolved in d₆-DMSO (2 mL) under an atmosphere of N₂. The mixture was heated to 80° C. and stirred for 18 h, then quenched with D₂O (2 mL) and diluted with DCM (20 mL). Saturated aqueous NH₄Cl solution (20 mL) was added and the layers separated. The aqueous phase was extracted with DCM (3×15 mL), the combined organic layers were washed with satd. Brine (20 mL), dried (NaSO₄), filtered and concentrated to give a semi-solid. This material was purified by column chromatography on silica gel, eluting with a gradient of MeOH in DCM to afford tert-butyl (6aR,9R)-9-(bis(ethyl-d₅)carbamoyl)-7-methyl-6a,7,8,9-tetrahydroindolo[4,3-fg]quinoline-4(6H)-carboxylate-5-d (56 mg, 94%) as an oil. m/z=435.30 [M+H]⁺; ¹H NMR (300 MHz, CDCl₃) δ 7.29 (m, 3H, 3×ArH), 6.29 (s, 1H, C═CH), 3.78 (m, 1H, CHCO), 3.42 (dd, 1H, J=15.1, 5.3 Hz, CHN), 3.10 (m, 1H, CHH), 2.98 (m, 1H, CHH), 2.79 (m, 1H, CHH), 2.51 (m, 4H, CHH and NCH₃), 1.59 (s, 9H, 3×CH₃).

Step 2: Preparation of (6aR,9R)—N,N-Bis(ethyl-d₅)-7-methyl-4,6,6a,7,8,9-hexahydroindolo[4,3-fg]quinoline-9-carboxamide-5-d trifluoroacetate-d

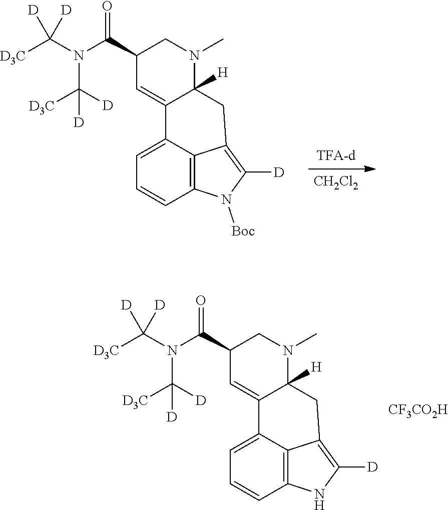

To a stirred solution of d₁₁-N-Boc-LSD (56 mg, 0.13 mmol, 1 equiv.) in DCM (0.55 mL) was added d-TFA (0.5 mL) dropwise. The mixture was stirred at rt for 1 h, then concentrated under vacuum and residual TFA was removed azeotropically with CHCl₃ (4×10 mL) to give a residue (49 mg). This material was purified by column chromatography on silica gel, eluting with a gradient of MeOH in DCM to afford (6aR,9R)—N,N-bis(ethyl-d₅)-7-methyl-4,6,6a,7,8,9-hexahydroindolo [4,3-fg]quinoline-9-carboxamide-5-d (10 mg, 17%) as a solid. m/z=335.25 [M+H]⁺; ¹H NMR (300 MHz, CDCl₃) δ 7.81 (s, 1H, NH), 7.13 (m, 3H, 3×ArH), 6.84 (s, 0.25H, ArH/D), 6.28 (s 1H, C═CH), 3.82 (m, 1H, CHCO), 3.49 (dd, 1H, J=14.6, 5.4 Hz, CHN), 3.19 (m, 1H, CHH), 2.99 (dd, 1H, J=10.9, 5.1 Hz, CHH), 2.83 (t, 1H, J=10.8 Hz, CHH), 2.61 (dd, 1H, J=14.5, 11.4 Hz, CHH), 2.53 (s, 3H, NCH₃).

### Example 8: Synthesis of (6aR,9R)—N,N-bis(ethyl-d₅)-7-(methyl-d₃)-4,6,6a,7,8,9-hexahydroindolo[4,3-fg]quinoline-9-carboxamide trifluoroacetate-d (Compound 50)

### Step 1: Preparation of tert-butyl (6aR,9R)-9-(Bis(ethyl-d₅)carbamoyl)-7-methyl-6a,7,8,9-tetrahydroindolo[4,3-fg]quinoline-4(6H)-carboxylate

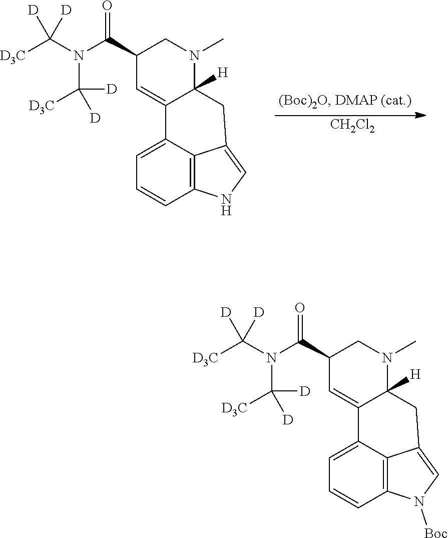 [SMILES: [2H]C([2H])([2H])C([2H])([2H])N(C(=O)[C@@H]1C=C2c3cccc4[nH]cc(c34)C[C@H]2N(C)C1)C([2H])([2H])C([2H])([2H])[2H] ; "CH2Cl2" ; [2H]C([2H])([2H])C([2H])([2H])N(C(=O)[C@@H]1C=C2c3cccc4c3c(cn4C(=O)OC(C)(C)C)C[C@H]2N(C)C1)C([2H])([2H])C([2H])([2H])[2H]]

d₁₀-LSD (110 mg, 0.33 mmol, 1 equiv.), di-tert-butyl carbonate (479 mg, 0.36 mmol, 1.1 equiv.) and DMAP (4 mg, 0.03 mmol, 0.1 equiv.) were dissolved in DCM (5 mL) and stirred at rt for 3 h. The mixture was diluted with DCM (20 mL) and H₂O (25 mL), the layers separated, and the aqueous phase was extracted with DCM (3×25 mL). The combined organic layers were washed with satd. brine, dried (MgSO₄), filtered and concentrated to give a semi-solid. This material was purified by column chromatography on silica gel, eluting with a gradient of MeOH in DCM to afford tert-butyl (6aR,9R)-9-(bis(ethyl-d₅)carbamoyl)-7-methyl-6a,7,8,9-tetrahydroindolo[4,3-fg]quinoline-4(6H)-carboxylate (135 mg, 95%) as a semi-solid. m/z=434.30 [M+H]⁺; ¹H NMR (300 MHz, CDCl₃) δ 7.72 (s, 1H, ArH), 7.25 (m, 3H, 3×ArH), 6.29 (s, 1H, ═CH), 3.79 (m, 1H, CHCO), 3.42 (dd, 1H, J=15.0, 5.4 Hz, CHN), 3.08 (m, 1H, CHH), 2.97 (ddd, 1H, J=11.4, 5.0, 1.2 Hz, CHH), 2.97 (t, 1H, J=10.9 Hz, CHH) 2.53 (m, 1H, CHH), 2.51 (s, 3H, NCH₃), 1.59 (s, 9H, 3×CH₃).

### Step 2: Preparation of tert-Butyl (6aR,9R)-9-(bis(ethyl-d₅)carbamoyl)-6a,7,8,9-tetrahydroindolo[4,3-fg]quinoline-4(6H)-carboxylate

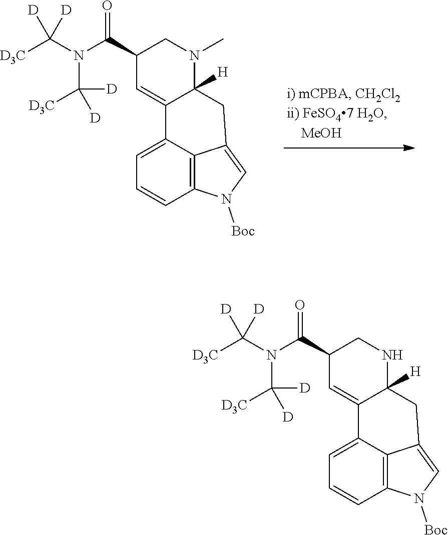 [SMILES: [2H]C([2H])([2H])C([2H])([2H])N(C(=O)[C@@H]1C=C2c3cccc4c3c(cn4C(=O)OC(C)(C)C)C[C@H]2N(C)C1)C([2H])([2H])C([2H])([2H])[2H] ; [2H]C([2H])([2H])C([2H])([2H])N(C(=O)[C@@H]1C=C2c3cccc4c3c(cn4C(=O)OC(C)(C)C)C[C@H]2NC1)C([2H])([2H])C([2H])([2H])[2H]]

To a solution of tert-butyl (6aR,9R)-9-(bis(ethyl-d₅)carbamoyl)-7-methyl-6a,7,8,9-tetrahydroindolo[4,3-fg]quinoline-4(6H)-carboxylate (131 mg, 0.31 mmol, 1 equiv.) in DCM (5 mL) at 0° C. was added portion-wise 3-chloroperbenzoic acid (64 mg, 0.37 mmol, 1.2 equiv.). The reaction mixture was stirred at 0° C. for 1 h at which point a suspension of iron(II) sulfate heptahydrate (48 mg, 0.17 mmol, 0.5 equiv.) in MeOH (1 mL) was added dropwise and the mixture was warmed to rt and stirred for 48 h. The reaction was quenched with satd. aqueous NaS₂O₃ solution (20 mL), the layers separated, and the aqueous phase was extracted with DCM (2×5 mL). The combined organic layers were washed with satd. aqueous NaHCO₃ solution (20 mL), dried (Na₂SO₄), filtered and concentrated to give a semi solid. This material was purified by column chromatography on silica gel, eluting with a gradient of MeOH in DCM to afford tert-butyl (6aR,9R)-9-(bis(ethyl-d₅)carbamoyl)-6a,7,8,9-tetrahydroindolo[4,3-fg]quinoline-4(6H)-carboxylate (43 mg, 33%) as a semi-solid. m/z=420.30 [M+H]⁺; ¹H NMR (300 MHz, CDCl₃) δ 7.95 (m, 2H, ArH, NH), 7.39 (m, 3H, 3×ArH), 6.45 (s, 1H, C═CH), 3.92 (m, 2H, CHCO and CHN), 3.41 (m, 1H, CHH), 3.19 (m, 2H, CHH and CHH), 2.75 (m, 1H, CHH), 1.69 (s, 9H, 3×CH₃).

### Step 3: Preparation of tert-butyl (6aR,9R)-9-(Bis(ethyl-d₅)carbamoyl)-7-(methyl-d₃)-6a,7,8,9-tetrahydroindolo-[43-fg]quinoline-4(6H)-carboxylate

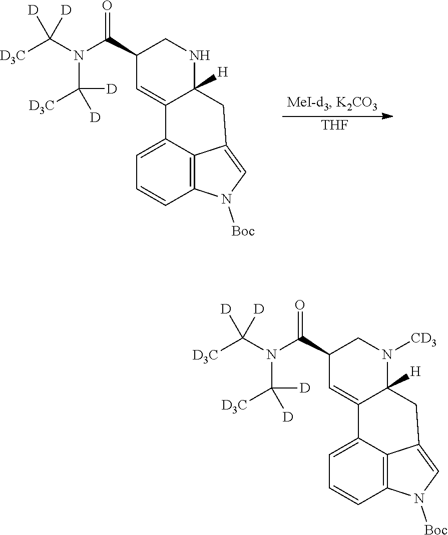 [SMILES: [2H]C([2H])([2H])C([2H])([2H])N(C(=O)[C@@H]1C=C2c3cccc4c3c(cn4C(=O)OC(C)(C)C)C[C@H]2NC1)C([2H])([2H])C([2H])([2H])[2H] ; [2H]C([2H])([2H])N1C[C@H](C(=O)N(C([2H])([2H])C([2H])([2H])[2H])C([2H])([2H])C([2H])([2H])[2H])C=C2c3cccc4c3c(cn4C(=O)OC(C)(C)C)C[C@H]21 ; "THF"]

To a solution of tert-butyl (6aR,9R)-9-(bis(ethyl-d₅)carbamoyl)-6a,7,8,9-tetrahydroindolo [4,3-fg]quinoline-4(6H)-carboxylate (42 mg, 0.1 mmol, 1 equiv.) in THE (3 mL) was added K₂CO₃ (13 mg, 0.10 mmol, 1 equiv.) and methyl iodide-d₃ (15.4 mg, 7 μL, 0.11 mmol, 1.1 equiv.). The mixture was heated to 60° C. and stirred for 16 h, then cooled to rt and diluted with DCM (15 mL) and H₂O (15 mL). The layers were separated and the aqueous layer extracted with DCM (3×15 mL). The combined organic layers were washed with satd. brine (20 mL), dried (MgSO₄), filtered and concentrated. This material was purified by column chromatography on silica gel, eluting with a gradient of MeOH in DCM to afford tert-butyl (6aR,9R)-9-(bis(ethyl-d₅)carbamoyl)-7-(methyl-d₃)-6a,7,8,9-tetrahydroindolo[4,3-fg]quinoline-4(6H)-carboxylate (16.2 mg, 37%) as a semi-solid. m/z=427.35 [M+H]⁺.

### Step 4: Preparation of (6aR,9R)—N,N-bis(ethyl-d₅)-7-(methyl-d₃)-4,6,6a,7,8,9-hexahydroindolo[4,3-fg]quinoline-9-carboxamide trifluoroacetate-d

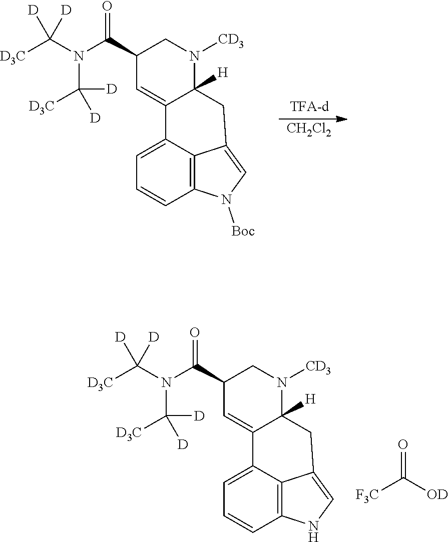

A solution of tert-butyl (6aR,9R)-9-(bis(ethyl-d₅)carbamoyl)-7-(methyl-d₃)-6a,7,8,9-tetrahydroindolo[4,3-fg]quinoline-4(6H)-carboxylate (16 mg, 0.036 mmol, 1 equiv.) in a 1:1 mixture of TFA-d:DCM (2 mL) was stirred at rt for 2 h. The mixture was concentrated and the residue was azeotroped with CHCl₃ (3×3 mL) to afford (6aR,9R)—N,N-bis(ethyl-d₅)-7-(methyl-d₃)-4,6,6a,7,8,9-hexahydroindolo[4,3-fg]quinoline-9-carboxamide trifluoroacetate-d (7 mg, 44%) as a semi-solid. m/z=337.30 [M+H]⁺; ¹H NMR (300 MHz, CDCl₃) δ 7.16 (d, 1, J=8.6 Hz, ArH), 7.10-6.83 (m, 3H, 3×ArH), 6.32 (s, 1H, C═CH), 4.33 (m, 1H, CHCO), 3.82 (m, 1H, CHN), 3.66 (m, 1H, CHH), 3.56 (m, 1H, CHH), 3.29 (m, 1H, CH, CHH), 2.99 (m, 1H, CH, CHH).

### Example 9: Synthesis of (6aR,9R)—N,N-diethyl-7-methyl-4,6,6a,7,8,9-hexahydroindolo[4,3-fg]quinoline-9-carboxamide-1,2,3,5-d₄ triflate-d (Compound 45)

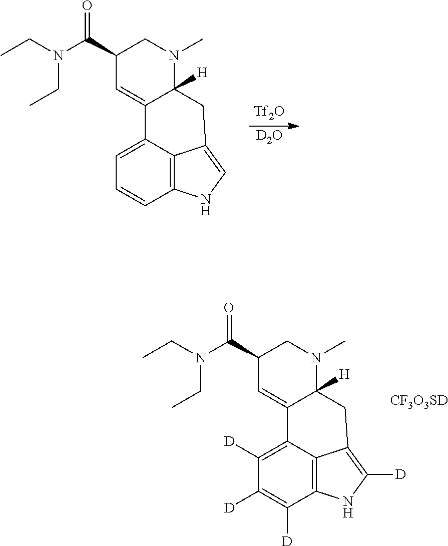

To trifluoromethanesulfonic anhydride (2.34 g, 1.40 mL, 8.3 mmol, 18 equiv.) was added D₂O (166 mg, 150 μL, 8.3 mmol, 18 equiv.) dropwise at 0° C. in a sealed microwave vessel under an atmosphere of N₂. The mixture was heated to 80° C. and stirred for 1 h, after which the layers combined. The mixture was cooled to rt and LSD (150 mg, 0.46 mmol, 1 equiv.) was added portion-wise and the mixture was stirred at rt for 1 h. The mixture was cooled to 0° C., diluted with DCM (20 mL), quenched with D₂O (2 mL) then satd. aqueous NaHCO₃ (15 mL) and the layers separated. The aqueous phase was extracted with DCM (3×25 mL), the combined organic layers were washed satd. brine (35 mL), dried (MgSO₄), filtered and concentrated to obtain a solid. This material was purified by column chromatography on silica gel, eluting with a gradient of MeOH in DCM to give (6aR,9R)—N,N-diethyl-7-methyl-4,6,6a,7,8,9-hexahydroindolo[4,3-fg]quinoline-9-carboxamide-1,2,3,5-d₄ triflate-d (75 mg, 34%) as a solid. m/z=326.20 [M+H]⁺; ¹H NMR (300 MHz, CDCl₃) δ 7.94 (s, 1H, NH), 7.20 (m, 1.9H, ArH/D), 6.91 (s, 0.3H, ArH/D), 6.35 (s, 1H, C═CH), 3.92 (m, 1H, CHCO), 3.60 (m, 1H, CHN), 3.45 (m, 4H, 2×CH₂), 3.26 (m, 1H, CHH), 3.13 (m, 1H CHH), 2.95 (t, 1H, J=10.8 Hz, CHH), 2.71 (m, 1H, CHH), 2.64 (s, 3H, CH₃), 1.28 (m, 6H, 2×CH₃).

### Example 10: Synthesis of (6aR,9R)—N,N-diethyl-7-methyl-4,6,6a,7,8,9-hexahydroindolo[4,3-fg]quinoline-9-carboxamide-4,5-d trifluoroacetate-d (Compound 41)

### Step 1: Preparation of tert-butyl (6aR,9R)-9-(diethylcarbamoyl)-7-methyl-6a,7,8,9-tetrahydroindolo[4,3-fg]quinoline-4(6H)-carboxylate-5-d

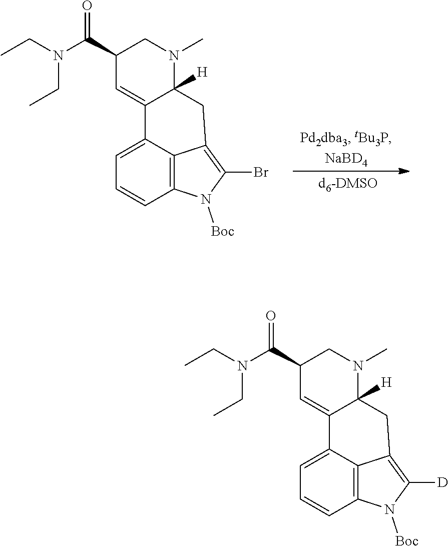 [SMILES: CCN(CC)C(=O)[C@@H]1C=C2c3cccc4c3c(c(Br)n4C(=O)OC(C)(C)C)C[C@H]2N(C)C1 ; [2H]c1c2c3c(cccc3n1C(=O)OC(C)(C)C)C1=C[C@@H](C(=O)N(CC)CC)CN(C)[C@@H]1C2]

2-Br—N-Boc-LSD (126 mg, 0.25 mmol, 1 equiv.), Pd₂(dba)₃ (4.8 mg, 0.005 mmol, 0.02 equiv.), NaBD₄ (22 mg, 0.53 mmol, 2.1 equiv.) and ^{t}Bu₃P (3.3 mg, 0.016 mmol, 0.06 equiv.) were dissolved in d₆-DMSO (1.2 mL) under an atmosphere of N₂ and heated to 80° C. and stirred for 18 h. The mixture was quenched with D₂O (2 mL) and saturated aqueous NH₄Cl (10 mL) and extracted with DCM (3×15 mL). The combined organic layers were washed with satd. brine (20 mL), dried (Na₂SO₄) and concentrated. The residue was purified by column chromatography on silica gel, eluting with a gradient of MeOH in DCM, to afford tert-butyl (6aR,9R)-9-(diethylcarbamoyl)-7-methyl-6a,7,8,9-tetrahydroindolo[4,3-fg]quinoline-4(6H)-carboxylate-5-d (43 mg, 40%) as a semi-solid. m/z=425.25 [M+H]⁺; ¹H NMR (CDCl₃, 300 MHz) δ 7.79 (s, 1H, ArH), 7.30 (m, 2H, obscured by solvent resonance, ArH), 6.35 (s, 1H, C═CH), 3.86 (m, 1H, CHCO), 3.46 (m, 5H, 2×CH₂Me and CHN), 3.15 (m, 1H, CHH), 3.04 (m, 1H, CHH), 2.86 (m, 1H, CHH), 2.57 (m, 4H, NCH₃ and CHH), 1.66 (s, 9H, 3×CH₃), 1.25 (m, 3H, CH₃), 1.17 (t, 3H, J=7.1 Hz, CH₃).

### Step 2: Preparation of (6aR,9R)—N,N-diethyl-7-methyl-4,6,6a,7,8,9-hexahydroindolo[4,3-fg]quinoline-9-carboxamide-4,5-d trifluoroacetate-d

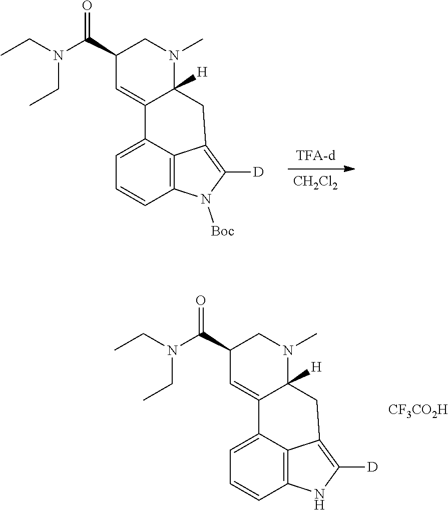

A solution of tert-butyl (6aR,9R)-9-(diethylcarbamoyl)-7-methyl-6a,7,8,9-tetrahydroindolo[4,3-fg]quinoline-4(6H)-carboxylate-5-d (40 mg, 0.10 mmol, 1 equiv.) in a 1:1 mixture of TFA-d:DCM (1 mL) was stirred at rt for 2 h. The mixture was concentrated and the residue was azeotroped with CHCl₃ (3 mL) to give 40 mg of a semi-solid residue. The residue was purified by column chromatography on silica gel, eluting with a gradient of MeOH in DCM, to give (6aR,9R)—N,N-diethyl-7-methyl-4,6,6a,7,8,9-hexahydroindolo[4,3-fg]quinoline-9-carboxamide-4,5-dtrifluoroacetate-d (10 mg, 24%) as a solid. m/z=325.25 [M+H]⁺; ¹HNMR (CDCl₃, 300 MHz) δ 7.91 (s, 1H, NH), 7.18 (m, 3H, 3×ArH), 6.35 (s, 1H, C═CH), 3.88 (dt, 1H, J=10.6, 5.3 Hz, CHCO), 3.56 (ddd, 1H, J=14.6, 5.5, 0.7 Hz, CHN), 3.46 (m, 4H, 2×CH₂Me), 3.23 (m, 1H, CHH), 3.06 (m, 1H, CHH), 2.90 (m, 1H, CHH), 2.68 (dd, 1H, J=14.5, 11.4 Hz, CHH), 2.60 (s, 3H, NCH₃), 1.25 (t, 3H, J=7.1 Hz, CH₃), 1.18 (t, 3H, J=7.1 Hz, CH₃).

### Example 11: Synthesis of tert-butyl (6aR,9R)-9-(diethylcarbamoyl)-7-(methyl-d₃)-6a,7,8,9-tetrahydroindolo[4,3-fg]quinoline-4(6H)-carboxylate (Compound 42)

### Step 1: Preparation of tert-Butyl (6aR,9R)-5-bromo-9-(diethylcarbamoyl)-6a,7,8,9-tetrahydroindolo[4,3-fg]quinoline-4(6H)-carboxylate

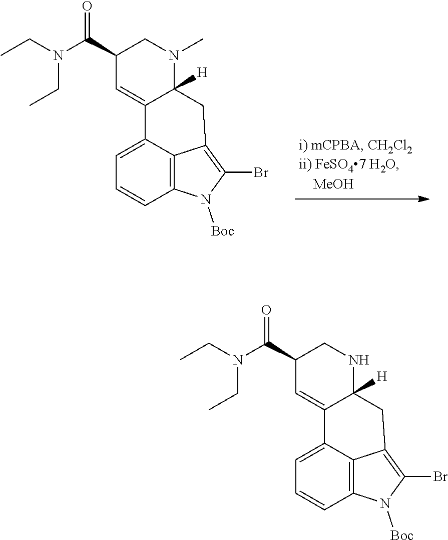 [SMILES: CCN(CC)C(=O)[C@@H]1C=C2c3cccc4c3c(c(Br)n4C(=O)OC(C)(C)C)C[C@H]2N(C)C1 ; CCN(CC)C(=O)[C@@H]1C=C2c3cccc4c3c(c(Br)n4C(=O)OC(C)(C)C)C[C@H]2NC1]

To a solution of 2-Br—N-Boc-LSD (1.39 g, 2.76 mmol, 1 equiv.) in DCM (20 mL) at 0° C. was added 3-chloroperbenzoic acid (0.57 g, 3.31 mmol, 1.2 equiv.) portion-wise and the mixture was stirred at rt for 1 h. A solution of iron(II) sulfate heptahydrate (384 mg, 1.38 mmol, 0.5 equiv.) in MeOH (11 mL) was added at rt and the mixture was stirred for 2.5 h. The mixture was quenched with saturated aqueous sodium thiosulfate (20 mL) and the aqueous layer was extracted with DCM (3×15 mL). The combined organic layers were washed with saturated aqueous NaHCO₃ (20 mL), dried (MgSO₄), filtered and concentrated to give a solid (1.05 g). The residue was purified by column chromatography on silica gel, eluting with a gradient of MeOH in DCM, to give tert-butyl (6aR,9R)-5-bromo-9-(diethylcarbamoyl)-6a,7,8,9-tetrahydroindolo[4,3-fg]quinoline-4(6H)-carboxylate (81 mg, 6%) as a semi-solid. m/z=488.15 and 490.15 [M+H]⁺; ¹H NMR (CDCl₃, 300 MHz) δ 7.79 (d, 1H, J=8.0 Hz, NH), 7.35 (m, 2H, 2×ArH), 7.23 (m, 1H, obscured by solvent resonance, ArH), 6.37 (s, 1H, C═CH), 3.93 (m, 1H, CHCO), 3.71 (m, 1H, CHN), 3.40 (m, 4H, 2×CH₂Me), 3.33 (d, 2H, J=6.7 Hz, CH₂) 3.17 (dd, 1H, J=15.4, 5.9 Hz, CHH), 2.63 (dd, 1H, J=15.5, 12.1 Hz, CHH), 1.69 (s, 9H, 3×CH₃), 1.27 (t, 3H, J=7.1 Hz, CH₃), 1.17 (t, 3H, J=7.1 Hz, CH₃).

### Step 2: Preparation of tert-butyl (6aR,9R)-5-bromo-9-(diethylcarbamoyl)-7-(methyl-d₃)-6a,7,8,9-tetrahydroindolo[4,3-fg]quinoline-4(6H)-carboxylate

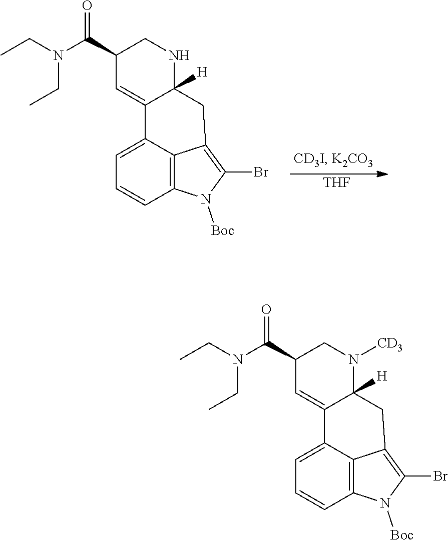 [SMILES: CCN(CC)C(=O)[C@@H]1C=C2c3cccc4c3c(c(Br)n4C(=O)OC(C)(C)C)C[C@H]2NC1 ; [2H]C([2H])([2H])N1C[C@H](C(=O)N(CC)CC)C=C2c3cccc4c3c(c(Br)n4C(=O)OC(C)(C)C)C[C@H]21 ; "THF"]

To a solution of tert-butyl (6aR,9R)-5-bromo-9-(diethylcarbamoyl)-6a,7,8,9-tetrahydroindolo[4,3-fg]quinoline-4(6H)-carboxylate (81 mg, 0.17 mmol, 1 equiv.) in anhydrous THE (3 mL) was added K₂CO₃ (23 mg, 0.17 mmol, 1 equiv.) and iodomethane-d₃ (27 mg, 11 μL, 0.18 mmol, 1.1 equiv.). The mixture was heated to 60° C. and stirred for 1.5 h, diluted with H₂O (10 mL) and extracted with DCM (3×5 mL). The combined organic layers were washed with satd. brine (5 mL), dried (Na₂SO₄), filtered and concentrated to give a semi-solid. The residue was purified by column chromatography on silica gel, eluting with a gradient of MeOH in DCM, to afford tert-butyl (6aR,9R)-5-bromo-9-(diethylcarbamoyl)-7-(methyl-d₃)-6a,7,8,9-tetrahydroindolo[4,3-fg]quinoline-4(6H)-carboxylate (29 mg, 35%) as a semi-solid. m/z=505.25 and 507.25 [M+H]⁺; ¹H NMR (CDCl₃, 300 MHz) δ 7.77 (d, 1H, J=8.2 Hz, ArH), 7.34 (m, 1H, ArH), 7.24 (m, 1H, obscured by solvent resonance, ArH), 6.35 (s, 1H, C═CH), 3.86 (m, 1H, CHCO), 3.41 (m, 5H, 2×CH₂Me and CHN), 3.16 (m, 1H, CHH), 3.04 (m, 1H, CHH), 2.85 (t, 1H, J=10.8 Hz, CHH), 2.44 (m, 1H, CHH), 1.69 (s, 9H, 3×CH₃), 1.26 (t, 3H, J=7.1 Hz, CH₃), 1.17 (t, 3H, J=7.1 Hz, CH₃).

### Step 3: Preparation of tert-butyl (6aR,9R)-9-(diethylcarbamoyl)-7-(methyl-d₃)-6a,7,8,9-tetrahydroindolo[4,3-fg]quinoline-4(6H)-carboxylate

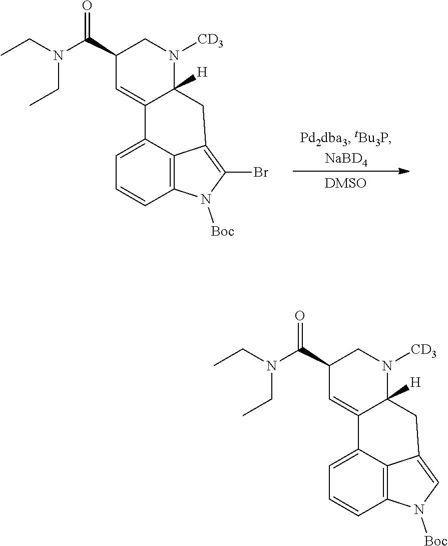 [SMILES: [2H]C([2H])([2H])N1C[C@H](C(=O)N(CC)CC)C=C2c3cccc4c3c(c(Br)n4C(=O)OC(C)(C)C)C[C@H]21 ; [2H]C([2H])([2H])N1C[C@H](C(=O)N(CC)CC)C=C2c3cccc4c3c(cn4C(=O)OC(C)(C)C)C[C@H]21]

tert-Butyl (6aR,9R)-5-bromo-9-(diethylcarbamoyl)-7-(methyl-d₃)-6a,7,8,9-tetrahydroindolo[4,3-fg]quinoline-4(6H)-carboxylate (29 mg, 0.057 mmol, 1 equiv.), Pd₂(dba)₃ (1.1 mg, 1.15 μmol, 0.02 equiv.), NaBH₄ (4.6 mg, 0.12 mmol, 2.1 equiv.) and ^{t}Bu₃P (0.7 mg, 3.44 μmol, 0.06 equiv.) were dissolved in anhydrous DMSO (2.2 mL) under an atmosphere of N₂, heated to 80° C. and stirred for 18 h. The mixture was quenched with H₂O (3 mL) and saturated aqueous NH₄Cl (10 mL) and extracted with DCM (3×8 mL). The combined organic layers were washed with satd. brine (15 mL), dried (MgSO₄), filtered and concentrated. The residue was purified by column chromatography on silica gel, eluting with a gradient of MeOH in DCM, to afford tert-butyl (6aR,9R)-9-(diethylcarbamoyl)-7-(methyl-d₃)-6a,7,8,9-tetrahydroindolo[4,3-fg]quinoline-4(6H)-carboxylate (5 mg, 21%) as a semi-solid. m/z=427.25 [M+H]⁺; ¹H NMR (CDCl₃, 300 MHz) δ 7.84 (m, 1H, ArH), 7.43 (dd, 1H, J=7.4, 0.8 Hz, ArH), 7.37 (d, 1H, J=7.8 Hz, ArH), 7.31 (m, 2H, ArH), 5.93 (s, 1H, C═CH), 3.37 (m, 7H, 2×CH₂Me, CHN, CHCO, and CHH), 3.19 (dd, 1H, J=11.3, 4.9 Hz, CHH), 3.02 (dd, 1H, J=15.5, 4.7 Hz, CHH), 2.75 (m, 1H, CHH), 1.66 (s, 9H, 3×CH₃), 1.10 (t, 6H, J=7.1 Hz, 2×CH₃).

### Step 4: Preparation of (6aR,9R)—N,N-diethyl-7-(methyl-d₃)-4,6,6a,7,8,9-hexahydroindolo[4,3-fg]quinoline-9-carboxamide trifluoroacetate

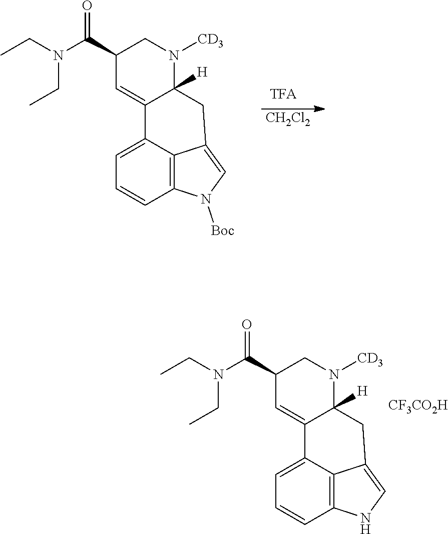

A solution of tert-butyl (6aR,9R)-9-(diethylcarbamoyl)-7-(methyl-d₃)-6a,7,8,9-tetrahydroindolo[4,3-fg]quinoline-4(6H)-carboxylate (5 mg, 0.012 mmol, 1 equiv.) in a 1:1 mixture of TFA:DCM (1.0 mL) was stirred at rt for 2 h. The mixture was concentrated and azeotroped with CHCl₃ (3 mL) to give (6aR,9R)—N,N-diethyl-7-(methyl-d₃)-4,6,6a,7,8,9-hexahydroindolo[4,3-fg]quinoline-9-carboxamide trifluoroacetate (4 mg) as a solid. m/z=327.25 [M+H]⁺.

### Prophetic Scheme for the Synthesis of Deuterated Versions of ALD-52, or Deuterated Versions of 1P-LSD:

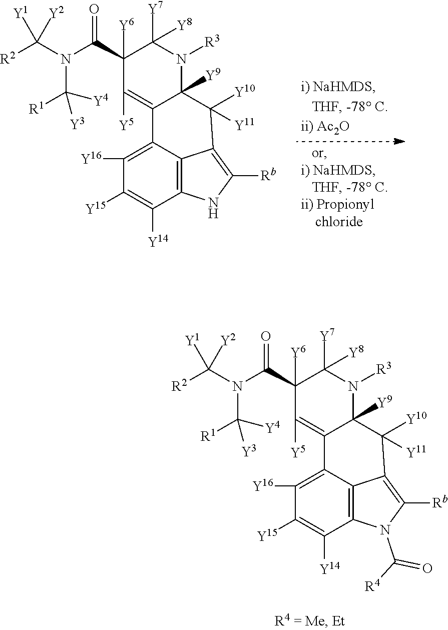

### Evaluation of Metabolic Stability in Human Liver Microsomes

### Protocol Summary

Test compound (1 μM) was incubated with pooled liver microsomes. Test compound was incubated at 5 time points over the course of a 45 min assay and the test compound was analyzed by LC-MS/MS.

### Objective

To determine the stability of the test compound in the presence of liver microsomes.

### Compound Requirements

Compound identifier, molecular weight and/or molecular formula.
50 μL of 10 mM test compound in DMSO per species per assay condition.

### Experimental Procedure

Pooled liver microsomes were purchased from a reputable commercial supplier. Microsomes were stored at −80° C. prior to use.

Microsomes (final protein concentration 0.5 mg/mL), 0.1 M phosphate buffer pH 7.4 and test compound (final substrate concentration 1 M; final DMSO concentration 0.25%) were preincubated at 37° C. prior to the addition of NADPH (final concentration 1 mM) to initiate the reaction. A minus cofactor control incubation was included for each compound tested where 0.1 M phosphate buffer pH 7.4 was added instead of NADPH (minus NADPH). Two control compounds were included with each species. All incubations were performed singularly for each test compound.

Each compound was incubated for 0, 5, 15, 30 and 45 min. The control (minus NADPH) was incubated for 45 min only. The reactions were stopped by transferring incubate into acetonitrile at the appropriate time points, in a 1:3 ratio. The termination plates were centrifuged at 3,000 rpm for 20 min at 4° C. to precipitate the protein.

### Quantitative Analysis

Following protein precipitation, the sample supernatants were combined in cassettes of up to 4 compounds, internal standard was added and samples analyzed using generic LCMS/MS conditions.

Optionally, if metabolite profiling was requested following the stability assay, a second assay was performed where the compound was incubated four times and the four resulting incubations were pooled to yield a higher sample concentration for analysis. The time point at which 30-70% of parent had degraded was then investigated at 3 different levels of metabolite profiling and/or identification.

### Data Analysis

From a plot of ln peak area ratio (compound peak area/internal standard peak area) against time, the gradient of the line was determined. Subsequently, half-life and intrinsic clearance were calculated using the equations below:

Elimination rate constant (k)=(−gradient)

Half-life(t1/2)(min)=0.693/k

Intrinsic clearance(CLint)(μL/min/mg protein)=(V×0.693)/t1/2

where V=Incubation volume(μL)/Microsomal protein (mg)

Relevant control compounds were assessed, ensuring intrinsic clearance values fell within the specified limits (if available). Any failures were rejected and the assay repeated.

The intrinsic clearance (CLint) of the test compound and t %/2 were returned.

In these experiments, values equal to or more than a 15% increase in half-life were considered to be a significant difference if the apparent intrinsic clearance ratio (isotopically enriched compound/2-Br-LSD or LSD) was >1.15 or <0.85, then there was considered to be significant differentiation.

TABLE 2
Metabolic stability in human liver microsomes of representative
deuterated 2-Br-LSD and LSD compounds.

| | | CLᵢₙₜ | |
|---|---|---|---|
| Compound | | (μL/min/mg | T_{1/2} |
| Number | Compound name | protein) | (min) |
| 1 | (6aR,9R)-5-bromo-N,N-diethyl-7- | 52.6 | 26.3 |
| | methyl-4,6,6a,7,8,9- | | |
| | hexahydroindolo[4,3-fg]quinoline-9- | | |
| | carboxamide-1,2,3-d₃ formate (2-Br- | | |
| | LSD-d₃ formate) | | |
| 2 | (6aR,9R)-5-bromo-N,N-diethyl-7- | 49.4 | 28.1 |
| | (methyl-d₃)-4,6,6a,7,8,9- | | |
| | hexahydroindolo[4,3-fg]quinoline-9- | | |
| | carboxamide (2-Br-LSD-d₃) | | |
| 7 | (6aR,9R)-5-bromo-N,N-bis(ethyl-d₅)- | 44.6 | 31.1 |
| | 7-methyl-4,6,6a,7,8,9- | | |
| | hexahydroindolo[4,3-fg]quinoline-9- | | |
| | carboxamide (2-Br-LSD-d₁₀) | | |
| 9 | (6aR,9R)-5-bromo-N,N-bis(ethyl-d₅)- | 38.8 | 35.7 |
| | 7-(methyl-d₃)-4,6,6a,7,8,9- | | |
| | hexahydroindolo[4,3-fg]quinoline-9- | | |
| | carboxamide trifluoroacetate-d (2-Br- | | |
| | LSD-d₁₃ TFA) | | |
| — | 2-Br-LSD | 56.3 | 24.6 |
| 41 | (6aR,9R)-N,N-Diethyl-7-methyl- | 17.8 | 77.7 |
| | 4,6,6a,7,8,9-hexahydroindolo[4,3- | | |
| | fg]quinoline-9-carboxamide-4,5-d | | |
| | trifluoroacetate-d (d1-LSD TFA) | | |
| 45 | (6aR,9R)-N,N-Diethyl-7-methyl- | 16.7 | 82.8 |
| | 4,6,6a,7,8,9-hexahydroindolo[4,3- | | |
| | fg]quinoline-9-carboxamide-1,2,3,5-d4 | | |
| | triflate-d (d4-LSD Triflate-d) | | |
| 47 | (6aR,9R)-N,N-Bis(ethyl-d5)-7-methyl- | 17.2 | 80.8 |
| | 4,6,6a,7,8,9-hexahydroindolo[4,3-fg] | | |
| | quinoline-9-carboxamide (d10-LSD) | | |
| 55 | (6aR,9R)-N,N-bis(ethyl-d5)-7-methyl- | 16.6 | 83.5 |
| | 4,6,6a,7,8,9-hexahydroindolo [4,3-fg] | | |
| | quinoline-9-carboxamide-5-d | | |
| | trifluroacetate-d (d11-LSD TFA-d) | | |
| 50 | (6aR,9R)-N,N-bis(ethyl-d5)-7- | 14.3 | 96.6 |
| | (methyl-d3)-4,6,6a,7,8,9- | | |
| | hexahydroindolo [4,3-fg]quinoline-9- | | |
| | carboxamide trifluroacetate-d (d13- | | |
| | LSD TFA-d) | | |
| 67 | (6aR,9R)-N,N-Bis(ethyl-d5)-7-methyl- | 11.5 | 120.0 |
| | 4,6,6a,7,8,9-hexahydroindolo[4,3-fg] | | |
| | quinoline-9-carboxamide-1,2,3,5-d4 | | |
| | triflate-d (d14-LSD Triflate-d) | | |
| — | LSD | 16.3 | 85.0 |
| — | dextromethorphan | 46.8 | 29.6 |
| — | verapamil * | 255 | 5.44 |

* 30 and 45 minute time points excluded because verapamil is metabolized very fast and is completely gone by 30 min. Thus, the 30 min and 45 min timepoints are not able to be measured.

Based on the results in Table 2, Compound (6aR,9R)-5-bromo-N,N-bis(ethyl-d₅)-7-methyl-4,6,6a,7,8,9-hexahydroindolo[4,3-fg]quinoline-9-carboxamide (compound 7) and Compound (6aR,9R)-5-bromo-N,N-bis(ethyl-d₅)-7-(methyl-d₃)-4,6,6a,7,8,9-hexahydroindolo[4,3-fg]quinoline-9-carboxamide trifluoroacetate-d (compound 9) exhibit significant differences in half-life and intrinsic clearance compared to 2-Br-LSD. Compounds 1 and 2 also exhibit differences in half-life and intrinsic clearance compared to 2-Br-LSD.

Compound (6aR,9R)—N,N-Bis(ethyl-d₅)-7-methyl-4,6,6a,7,8,9-hexahydroindolo[4,3-fg]quinoline-9-carboxamide-1,2,3,5-d₄ triflate-d (compound 67) exhibits significant differences in half-life and intrinsic clearance compared to LSD. Compounds 41, 45, 47, 50, and 55 also exhibit differences in half-life and intrinsic clearance compared to LSD.

Oral Bioavailability in Rats—Pharmacokinetics of test articles following a single intravenous or oral administration in rats: A pharmacokinetic (PK) study is performed in three male Sprague-Dawley (SD) rats following intravenous (IV) and oral (PO) administration of 2-bromo-LSD, LSD, ALD-52 or 1P-LSD or test deuterated/2-bromo-LSD, LSD, ALD-52 or 1P-LSD, at 1 mg/kg (IV) and 10 (PO) mg/kg. Test compounds, or 2-bromo-LSD, LSD, ALD-52 or 1P-LSD, are measured in plasma.

A detailed description of the in vivo methods:

### Rat Strain

Rats used in these studies are supplied by Charles River (Margate UK) and are specific pathogen free. The strain of rats is Sprague Dawley. Male rats are 175-225 g on receipt and are allowed to acclimatise for 5-7 days.

### Animal Housing

Rats are group housed in sterilised individual ventilated cages that expose the animals at all times to HEPA filtered sterile air. Animals will have free access to food and water (sterile) and will have sterile aspen chip bedding (at least once weekly). The room temperature is 22° C.+/−1° C., with a relative humidity of 60% and maximum background noise of 56 dB. Rats are exposed to 12-hour light/dark cycles.

### Treatment

### Treatment

Test article is diluted 10% v/v DMSO, 40% v/v PEG-400, 50% v/v Water. The test articles are administered in a dose volume of 2 mL/kg for intravenous (IV) and 5 mL/kg (PO) for oral routes of administration.

### Single IV/PO Dose Pharmacokinetics Study in Rats

Each test article is administered as a single IV bolus (via a lateral tail-vein) or a single oral gavage in cohorts of 3 rats per route. Following dose administrations, a 100 μL whole blood sample (EDTA) is collected via the tail-vein at time-points described in Table 3. The blood will be centrifuged to separate plasma. Approximately 40 μL of plasma is dispensed per time-point, per rat, in a 96 well plate and frozen until analysis. Bioanalysis will be carried out on plasma samples.

TABLE 3
Single IV and oral dose pharmacokinetics profile of test articles in rat plasma

| | | | Dose | | No. of |
|---|---|---|---|---|---|
| Group | Test article | Route | (mg/kg) | Blood sample collection (post dose) | rats |
| 1 | 2-Br-LSD | IV | 1 | 5 min, 15 min, 30 min, 1 h, 2 h, 4 h, 7 h, 24 h | 3 |
| 2 | 2-Br-LSD | PO | 10 | 15 min, 30 min, 45 min, 1 h, 2 h, 4 h, 7 h, 24 h | 3 |
| 3 | Test Article | IV | 1 | 5 min, 15 min, 30 min, 1 h, 2 h, 4 h, 7 h, 24 h | 3 |
| 4 | Test Article | PO | 10 | 15 min, 30 min, 45 min, 1 h, 2 h, 4 h, 7 h, 24 h | 3 |
| 5 | LSD | IV | 1 | 5 min, 15 min, 30 min, 1 h, 2 h, 4 h, 7 h, 24 h | 3 |
| 6 | LSD | PO | 10 | 15 min, 30 min, 45 min, 1 h, 2 h, 4 h, 7 h, 24 h | 3 |
| 7 | Test Article | IV | 1 | 5 min, 15 min, 30 min, 1 h, 2 h, 4 h, 7 h, 24 h | 3 |
| 8 | Test Article | PO | 10 | 15 min, 30 min, 45 min, 1 h, 2 h, 4 h, 7 h, 24 h | 3 |
| 9 | ALD-52 | IV | 1 | 5 min, 15 min, 30 min, 1 h, 2 h, 4 h, 7 h, 24 h | 3 |
| 10 | ALD-52 | PO | 10 | 15 min, 30 min, 45 min, 1 h, 2 h, 4 h, 7 h, 24 h | 3 |
| 11 | Test Article | IV | 1 | 5 min, 15 min, 30 min, 1 h, 2 h, 4 h, 7 h, 24 h | 3 |
| 12 | Test Article | PO | 10 | 15 min, 30 min, 45 min, 1 h, 2 h, 4 h, 7 h, 24 h | 3 |
| 13 | 1P-LSD | IV | 1 | 5 min, 15 min, 30 min, 1 h, 2 h, 4 h, 7 h, 24 h | 3 |
| 14 | 1P-LSD | PO | 10 | 15 min, 30 min, 45 min, 1 h, 2 h, 4 h, 7 h, 24 h | 3 |
| 15 | Test Article | IV | 1 | 5 min, 15 min, 30 min, 1 h, 2 h, 4 h, 7 h, 24 h | 3 |
| 16 | Test Article | PO | 10 | 15 min, 30 min, 45 min, 1 h, 2 h, 4 h, 7 h, 24 h | 3 |

### Dose Formulation Samples

Dose formulation samples are diluted in two steps with 50:50 (v/v) methanol/water to an appropriate concentration, then diluted 10:90 (v/v) with control matrix to match to the calibration standard in plasma.

### Sample Extraction Procedure

Calibration and QC standards, incurred samples, blank matrix and dose formulation samples are extracted by protein precipitation, via the addition of a bespoke acetonitrile (ACN)-based Internal Standard (IS) solution, containing several compounds and including Metoprolol and Rosuvastatin, both of which were monitored for during analysis. Following centrifugation, a 40 μL aliquot of supernatant is diluted by the addition of 80 μL water. The prepared sample extracts are analysed by LC-MS/MS.

### Example of Bioanalytical Method and Assay

| | |
|---|---|
| 1 | According to the plate layout, aliquot to wells in 0.8 mL |
| | 96-well plate (Abgene). 30 μL for Calibration, QC standards, |
| | blanks and dose formulation check. |
| 2 | Prepare Calibration and QC standards according to the assay |
| | information. Dilute dose formulation according to the assay |
| | information. Aliquot incurred samples according to the |
| | plate layout & assay information. |
| 3 | Add 90 μL of ACN internal standard and |
| | vortex mix for 5 minutes at 850 rpm |
| 4 | Centrifuge at nominally 4000 rpm for 10 minutes |
| 6 | Transfer 40 μL of supernatant into a new 0.8 mL Abgene plate. |
| 6 | Add 80 μL of water to all transferred supernatant. |
| 7 | Vortex mix for 30 seconds at 1400 rpm |
| 8 | Analyse immediately by LC-MS/MS or store at +4° C. until analysis. |

### Biological Assays and Methods

Head-Twitch Response (HTR). The head-twitch response assay is performed as is known to those of skill in the art using both male and female C₅₇BL/6J mice (2 per treatment). The mice are obtained and are approximately 8 weeks old at the time of the experiments. Compounds are administered via intraperitoneal injection (5 mL/kg) using 0.9% saline as the vehicle. As a positive control, LSD (0.32 mg/kg) is utilized. Behavior is videotaped, later scored by two blinded observers, and the results are averaged (Pearson correlation coefficient=0.93).

Serotonin and Opioid Receptor Functional Assays. Functional assay screens at 5-HT and opioid receptors are performed in parallel using the same compound dilutions and 384-well format high-throughput assay platforms. Assays assess activity at all human isoforms of the receptors, except where noted for the mouse 5-HT_{2A} receptor. Receptor constructs in pcDNA vectors were generated from the Presto-Tango GPCR library with minor modifications. All compounds were serially diluted in drug buffer (HBSS, 20 mM HEPES, pH 7.4 supplemented with 0.1% bovine serum albumin and 0.01% ascorbic acid) and dispensed into 384-well assay plates using a FLIPR^{TETRA} (Molecular Devices). Every plate included a positive control such as 5-HT (for all 5-HT receptors), DADLE (DOR), salvinorin A (KOR), and DAMGO (MOR). For measurements of 5-HT2A, 5-HT2B, and 5-HT2C Gq-mediated calcium flux function, HEK Flp-In 293 T-Rex stable cell lines (Invitrogen) were loaded with Fluo₋₄ dye for one hour, stimulated with compounds and read for baseline (0-10 seconds) and peak fold-over-basal fluorescence (5 minutes) at 25° C. on the FLIPR^{TETRA}. For measurement of 5-HT6 and 5-HT7a functional assays, Gs-mediated cAMP accumulation was detected using the split-luciferase GloSensor assay in HEKT cells measuring luminescence on a Microbeta Trilux (Perkin Elmer) with a 15 min drug incubation at 25° C. For 5-HT1A, 5-HT1B, 5-HT1F, MOR, KOR, and DOR functional assays, Gi/o-mediated cAMP inhibition was measured using the split-luciferase GloSensor assay in HEKT cells, conducted similarly as above, but in combination with either 0.3 μM isoproterenol (5-HT1A, 5-HT1B, 5-HT1F) or 1 μM forskolin (MOR, KOR, and DOR) to stimulate endogenous cAMP accumulation. For measurement of 5-HT1D, 5-HT1E, 5-HT4, and 5-HT5A functional assays, P-arrestin2 recruitment was measured by the Tango assay utilizing HTLA cells expressing TEV fused-P-arrestin2, as described previously with minor modifications. Data for all assays were plotted and non-linear regression was performed using “log(agonist) vs. response” in Graphpad Prism to yield Emax and EC₅₀ parameter estimates.

5HT_{2A} Sensor Assays. HEK293T (ATCC) 5HT_{2A} sensor stable line (sLight1.3s) is generated via lentiviral transduction of HIV-EF1α-sLight1.3 and propagated from a single colony. Lentivirus is produced using 2^{nd} generation lentiviral plasmids pHIV-EF1α-sLight1.3, pHCMV-G, and pCMV-deltaR8.2.

For the screening of the compounds, sLight1.3s cells are plated in 96-well plates at a density of 40000 24-hours prior to imaging. On the day of imaging, compounds solubilized in DMSO are diluted from the 100 mM stock solution to working concentrations of 1 mM, 100 mM and 1 μM with a DMSO concentration of 1%. Immediately prior to imaging, cells growing in DMEM (Gibco) are washed 2× with HBSS (Gibco) and in agonist mode 180 μL of HBSS or in antagonist mode 160 μL of HBSS is added to each well after the final wash. For agonist mode, images are taken before and after the addition of the 20 μL compound working solution into the wells containing 180 μL HBSS. This produces final compound concentrations of 100 mM, 10 mM and 100 nM with a DMSO concentration of 0.1%. For antagonist mode, images are taken before and after addition of 20 μL of 900 nM 5-HT and again after 20 μL of the compound working solutions to produce final concentrations of 100 nM for 5HT and 100 mM, 10 mM and 100 nM for the compounds with a DMSO concentration of 0.1%. Each compound is tested in triplicate (3 wells) for each concentration (100 mM, 10 mM and 100 nM). Additionally, within each plate, 100 nM 5HT and 0.1% DMSO controls are also imaged.

Imaging is performed using the Leica DMi8 inverted microscope with a 40× objective using the FITC preset with an excitation of 460 nm and emission of 512-542 nm. For each well, the cellular membrane where the 5HT_{2A} sensor is targeted is autofocused using the adaptive focus controls and 5 images from different regions within the well were taken with each image processed from a 2×2 binning.

For data processing, the membranes from each image are segmented and analyzed using a custom algorithm written in MATFAB producing a single raw fluorescence intensity value. For each well the 5 raw fluorescence intensity values generated from the 5 images are averaged and the change in fluorescence intensity (dFF) is calculated as:

dFF=(Fₛₐₜ−Fₐₚₒ)/Fₐₚₒ

For both agonist and antagonist modes, the fluorescence intensity values before compound addition in FIBSS only are used as the Fₐₚₒ values while the fluorescence intensity values after compound addition are used as the Fₛₐₜ values.

For agonist mode, data are as percent activation relative to 5HT, where 0 is the average of the DMSO wells and 100 is the average of the 100 μM 5HT wells. For antagonist mode, the inactivation score is calculated as:

Inactivation score=(dFFF(Compound+5HT)−dFF(5HT))/dFF(5HT)

Plasticity Effects: Treatment of rat embryonic cortical neurons with compounds of Formulas (A), (I), (II), (IIa), (IIb), (IIc), and Table 1 is evaluated for increased dendritic arbor complexity at 6 days in vitro (DIV6) as measured by Sholl analysis. The effect of the present compounds on dendritic growth can be determined to be 5-HT_{2A}-dependent, if pretreatment with ketanserin—a 5-HT_{2A} antagonist—inhibits their effects.

In addition to promoting dendritic growth, the present compounds also are evaluated for increased dendritic spine density to a comparable extent as ibogaine in mature cortical cultures (DIV20). The effects of the compounds on cortical dendritic spine dynamics in vivo using transcranial 2-photon imaging is assessed. First, spines are imaged on specific dendritic loci defined by their relation to blood vessel and dendritic architectures. Next, the animals are systemically administered vehicle, a compound of the present invention, or the hallucinogenic 5-HT_{2A} agonist 2,5-dimethoxy-4-iodoamphetamine (DOI). After 24 h, the same dendritic segments are re-imaged, and the number of spines gained or lost is quantified. Examples of the presently disclosed compounds increase spine formation in mouse primary sensory cortex, suggesting that the present compounds support neuronal plasticity.

As increased cortical structural plasticity in the anterior parts of the brain mediates the sustained (>24 h) antidepressant-like effects of ketamine and play a role in the therapeutic effects of 5-HT_{2A} agonists, the impact of the present compounds on forced swim test (FST) behavior is evaluated. First, a pretest is used to induce a depressive phenotype. Compounds are administered 24 h after the pre-test, and the FST is performed 24 h and 7 d post compound administration. Effective compounds of the invention, like ketamine, significantly reduced immobility 24 h after administration.

Dendritogenesis Assays. Compounds disclosed herein are evaluated for their ability to increase dendritic arbor complexity in cultures of cortical neurons using a phenotypic assay. Following treatment, neurons are fixed and visualized using an antibody against MAP2—a cytoskeletal protein localized to the somatodendritic compartment of neurons. Sholl analysis is then performed, and the maximum number of crossings (Nₘₐₓ) was used as a quantitative metric of dendritic arbor complexity. For statistical comparisons between specific compounds, the raw Nₘₐₓ values are compared. Percent efficacies are determined by setting the Nₘₐₓ values for the vehicle (DMSO) and positive (ketamine) controls equal to 0% and 100%, respectively.

Animals. For the dendritogenesis experiments, timed pregnant Sprague Dawley rats are obtained. For the head-twitch response assay, male and female C₅₇BL/6J mice are obtained.

Dendritogenesis—Sholl Analysis. Dendritogenesis experiments are performed following a previously published methods with slight modifications. Neurons are plated in 96-well format (200 μL of media per well) at a density of approximately 15,000 cells/well in Neurobasal (Life Technologies) containing 1% penicillin-streptomycin, 10% heat-inactivated fetal bovine serum, and 0.5 mM glutamine. After 24 h, the medium is replaced with Neurobasal containing 1× B27 supplement (Life Technologies), 1% penicillin-streptomycin, 0.5 mM glutamine, and 12.5 μM glutamate. After 3 days in vitro (DIV3), the cells are treated with compounds. All compounds tested in the dendritogenesis assays are treated at 10 μM. Stock solutions of the compounds in DMSO are first diluted 100-fold in Neurobasal before an additional 10-fold dilution into each well (total dilution=1:1000; 0.1% DMSO concentration). Treatments are randomized. After 1 h, the media is removed and replaced with new Neurobasal media containing 1× B27 supplement, 1% penicillin-streptomycin, 0.5 mM glutamine, and 12.5 mM glutamate. The cells are allowed to grow for an additional 71 h. At that time, neurons are fixed by removing 80% of the media and replacing it with a volume of 4% aqueous paraformaldehyde (Alfa Aesar) equal to 50% of the working volume of the well. Then, the cells are incubated at room temperature for 20 min before the fixative is aspirated and each well washed twice with DPBS. Cells are permeabilized using 0.2% Triton X-100 (ThermoFisher) in DPBS for 20 minutes at room temperature without shaking. Plates are blocked with antibody diluting buffer (ADB) containing 2% bovine serum albumin (BSA) in DPBS for 1 h at room temperature. Then, plates are incubated overnight at 4° C. with gentle shaking in ADB containing a chicken anti-MAP2 antibody (1:10,000; EnCor, CPCA-MAP2). The next day, plates are washed three times with DPBS and once with 2% ADB in DPBS. Plates are incubated for 1 h at room temperature in ADB containing an anti-chicken IgG secondary antibody conjugated to Alexa Fluor 488 (Life Technologies, 1:500) and washed five times with DPBS. After the final wash, 100 μL of DPBS is added per well and imaged on an ImageXpress Micro XL High-Content Screening System (Molecular Devices, Sunnyvale, CA) with a 20× objective. Images are analyzed using ImageJ Fiji (version 1.51 W). First, images corresponding to each treatment are sorted into individual folders that are then blinded for data analysis. Plate controls (both positive and negative) are used to ensure that the assay is working properly as well as to visually determine appropriate numerical values for brightness/contrast and thresholding to be applied universally to the remainder of the randomized images. Next, the brightness/contrast settings are applied, and approximately 1-2 individual pyramidal-like neurons per image (i.e., no bipolar neurons) are selected using the rectangular selection tool and saved as separate files. Neurons are selected that do not overlap extensively with other cells or extend far beyond the field of view.

In Vivo Spine Dynamics. Male and female Thyl-GFP-M line mice (n=5 per condition) are purchased from The Jackson Laboratory (JAX #007788) and maintained. In vivo transcranial two-photon imaging and data analysis are performed as previously described. Briefly, mice are anesthetized with an intraperitoneal (i.p.) injection of a mixture of ketamine (87 mg/kg) and xylazine (8.7 mg/kg). A small region of the exposed skull is manually thinned down to 20-30 pm for optical access. Spines on apical dendrites in mouse primary sensory cortices are imaged using a Bruker Ultima IV two-photon microscope equipped with an Olympus water-immersion objective (40×, NA=0.8) and a Ti:Sapphire laser (Spectra-Physics Mai-Tai, excitation wavelength 920 nm). Images are taken at a zoom of 4.0 (pixel size 0.143×0.143 pm) and Z-step size of 0.7 pm. The mice receive an i.p. injection (injection volume=5 mL/kg) of DOI (10 mg/kg) or test compound (50 mg/kg) immediately after they recover from anesthesia given prior to the first imaging session. The animals are re-imaged 24 h after drug administration. Dendritic spine dynamics were analyzed using ImageJ. Spine formation and elimination were quantified as percentages of spine number on day 0.

Forced Swim Test (FST). Male C₅₇/BL6J mice (9-10 weeks old at time of experiment) are obtained. After 1 week in the vivarium each mouse is handled for approximately 1 minute by the experimenter for 3 consecutive days leading up to the first FST. All experiments are carried out by the same experimenter who performs handling. During the FST, mice undergo a 6 min swim session in a clear Plexiglas cylinder 40 cm tall, 20 cm in diameter, and filled with 30 cm of 24±1° C. water. Fresh water is used for every mouse. After handling and habituation to the experimenter, drug-naive mice first undergo a pretest swim to more reliably induce a depressive phenotype in the subsequent FST sessions. Immobility scores for all mice are determined after the pre-test and mice are randomly assigned to treatment groups to generate groups with similar average immobility scores to be used for the following two FST sessions. The next day, the animals receive intraperitoneal injections of experimental compounds (20 mg/kg), a positive control (ketamine, 3 mg/kg), or vehicle (saline). The animals were subjected to the FST 30 mins after injection and then returned to their home cages. All FSTs are performed between the hours of 8 am and 1 pm. Experiments are video-recorded and manually scored offline. Immobility time defined as passive floating or remaining motionless with no activity other than that needed to keep the mouse's head above water is scored for the last 4 min of the 6 min trial.

Statistical analysis. Treatments are randomized, and data are analyzed by experimenters blinded to treatment conditions. Statistical analyses are performed using GraphPad Prism (version 8.1.2). The specific tests are, F-statistics and degrees of freedom. All comparisons are planned prior to performing each experiment. For dendritogenesis experiments a one way ANOVA with Dunnett's post hoc test is deemed most appropriate. Ketamine was included as a positive control to ensure that the assay is working properly.

Alcohol Use Disorder Model: To assess the anti-addictive potential of the present compounds, an alcohol drinking paradigm that models heavy alcohol use and binge drinking behavior in humans is employed. Using a 2-bottle choice setup (20% ethanol (v/v), EtOH vs. water, H₂O), mice are subjected to repeated cycles of binge drinking and withdrawal over the course of 7 weeks.

This schedule results in heavy EtOH consumption, binge drinking-like behavior, and generates blood alcohol content equivalent to that of human subjects suffering from alcohol use disorder (AUD). Next, compounds of the invention are administered via intraperitoneal injection 3 h prior to a drinking session, and EtOH and H₂O consumption is monitored. Effective compounds of the invention robustly reduce binge drinking during the first 4 h, decreasing EtOH consumption. With exemplary compounds, consumption of ethanol is lower for at least two days following administration with no effect on water intake. Efficacy in this assay suggests the present compounds are useful for the treatment of AUD.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

We claim:

1. A compound of Formula (A) 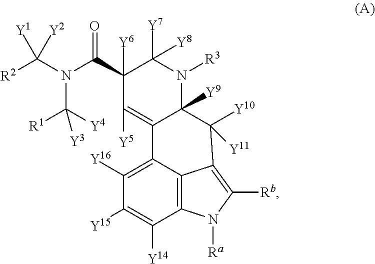
or a pharmaceutically acceptable salt thereof, wherein:
R¹, R², and R³ are methyl;
R^{a} is hydrogen, deuterium, tritium, acetyl, or 1-propionyl;
R^{b} is hydrogen, deuterium, tritium, or bromo;
Y¹, Y², Y³, Y⁴, Y⁵, Y⁶, Y⁷, Y⁸, Y⁹, Y¹⁰, Y¹¹, Y¹⁴, Y¹⁵, and Y¹⁶ are independently selected from hydrogen, deuterium, and tritium; and
at least one of R¹, R², R³, R^{a}, R^{b}, Y¹, Y², Y³, Y⁴, Y⁵, Y⁶, Y⁷, Y⁸, Y⁹, Y¹⁰, Y¹¹, Y¹⁴, Y¹⁵, and Y¹⁶ is or comprises deuterium or tritium.

2. The compound of claim 1, wherein at least one of R¹, R², R³, R^{a}, R^{b}, Y¹, Y², Y³, Y⁴, Y⁵, Y⁶, Y⁷, Y⁸, Y⁹, Y¹⁰, Y¹¹, Y¹⁴, Y¹⁵, and Y¹⁶ is or comprises deuterium.

3. The compound of claim 1 or 2, wherein R¹, R², and R³ are independently selected from CD₃, CD₂H, CDH₂, CT₃, CT₂H, CTH₂ and CH₃.

4. The compound of any one of claims 1-3, wherein R¹, R², and R³ are independently selected from CD₃, CD₂H, CDH₂, and CH₃.

5. The compound of any one of claims 1-4, wherein at least one of R^{a}, R^{b}, Y¹, Y², Y³, Y⁴, Y⁵, Y⁶, Y⁷, Y⁸, Y⁹, Y¹⁰, Y¹¹, Y¹⁴, Y¹⁵, and Y¹⁶ is deuterium.

6. The compound of any one of claims 1-5, wherein the compound is of Formula (I): 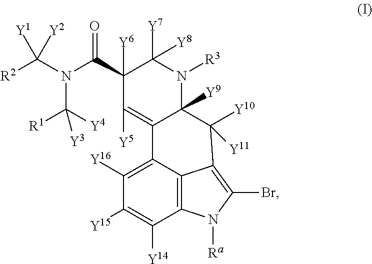
or a pharmaceutically acceptable salt thereof, wherein:
R¹, R², and R³ are methyl;
Y¹, Y², Y³, Y⁴, Y⁵, Y⁶, Y⁷, Y⁸, Y⁹, Y¹⁰, Y¹¹, R^{a}, Y¹⁴, Y¹⁵ and Y¹⁶ each are hydrogen or deuterium.

7. The compound of claim 6, wherein at least one of R¹, R², R³, R^{a}, Y¹, Y², Y³, Y⁴, Y⁵, Y⁶, Y⁷, Y⁸, Y⁹, Y¹⁰, Y¹¹, Y¹⁴, Y¹⁵, and Y¹⁶ is or comprises deuterium.

8. The compound of claim 6 or 7, wherein R¹, R², and R³ are independently selected from CD₃, CD₂H, CDH₂, CT₃, CT₂H, CTH₂ and CH₃.

9. The compound of any one of claims 6-8, wherein R¹, R², and R³ are independently selected from CD₃, CD₂H, CDH₂, and CH₃.

10. The compound of any one of claims 6-9, wherein at least one of R^{a}, Y¹, Y², Y³, Y⁴, Y⁵, Y⁶, Y⁷, Y⁸, Y⁹, Y¹⁰, Y¹¹, Y¹⁴, Y¹⁵, and Y¹⁶ is deuterium.

11. The compound of any one of claims 6-10, wherein the compound is selected from the group consisting of:

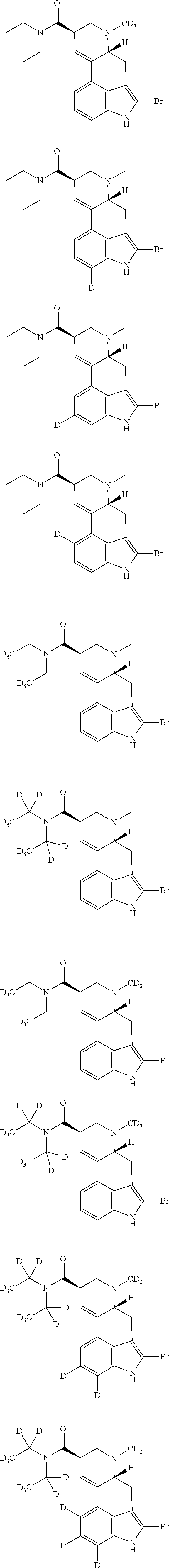

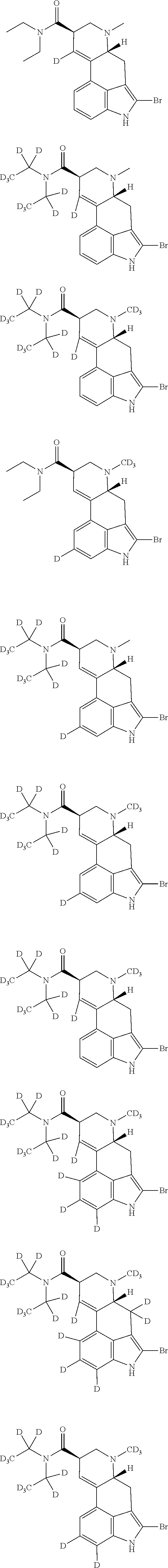

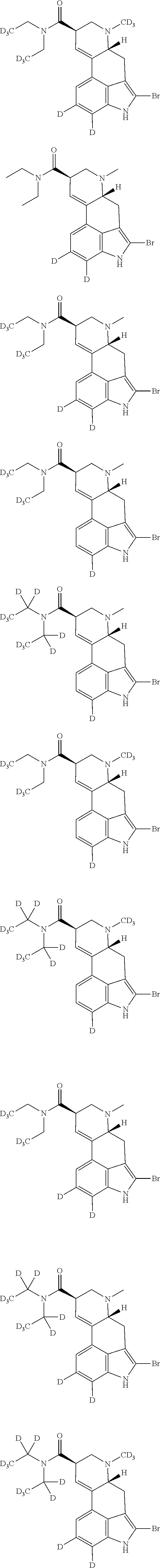

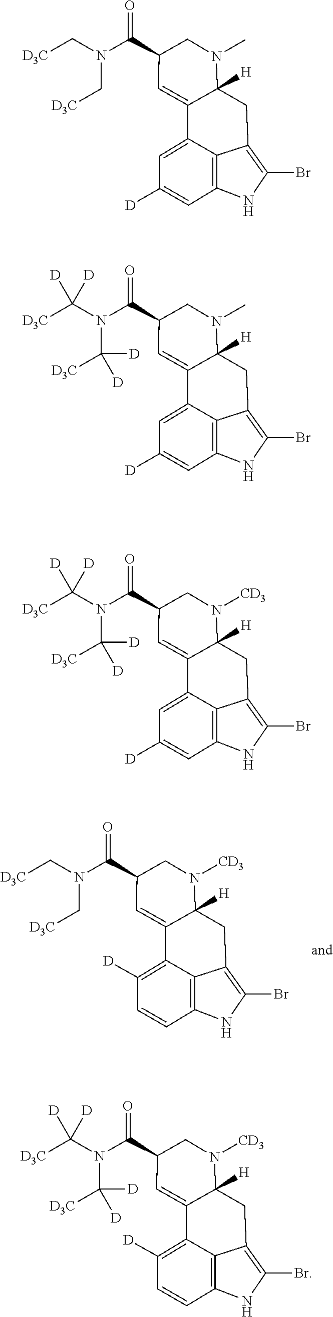

12. The compound of any one of claims 1-11, wherein the compound is of Formula (II): 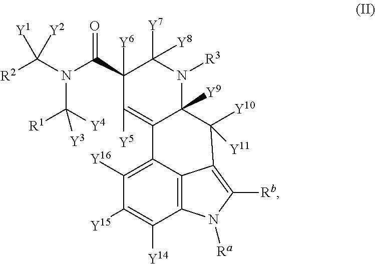
wherein
R¹, R² and R³ are methyl;
R^{b}, Y¹, Y², Y³, Y⁴, Y⁵, Y⁶, Y⁷, Y⁸, Y⁹, Y¹⁰, Y¹¹, Y¹⁴, Y¹⁵ and Y¹⁶ each are hydrogen or deuterium; and
R^{a} is selected from the group consisting of hydrogen, deuterium, acetyl and 1-propionyl.

13. The compound of claim 12, wherein at least one of R¹, R², R³, R^{a}, R^{b}, Y¹, Y², Y³, Y⁴, Y⁵, Y⁶, Y⁷, Y⁸, Y⁹, Y¹⁰, Y¹¹, Y¹⁴, Y¹⁵, and Y¹⁶ is or comprises deuterium.

14. The compound of claim 12 or 13, wherein R¹, R², and R³ are independently selected from CD₃, CD₂H, CDH₂, CT₃, CT₂H, CTH₂ and CH₃.

15. The compound of any one of claims 12-14, wherein R¹, R², and R³ are independently selected from CD₃, CD₂H, CDH₂, and CH₃.

16. The compound of any one of claims 12-15, wherein at least one of R^{a}, R^{b}, Y¹, Y², Y³, Y⁴, Y⁵, Y⁶, Y⁷, Y⁸, Y⁹, Y¹⁰, Y¹¹, Y¹⁴, Y¹⁵, and Y¹⁶ is deuterium.

17. The compound of any one of claims 1-16, wherein the compound is of Formula (IIa): 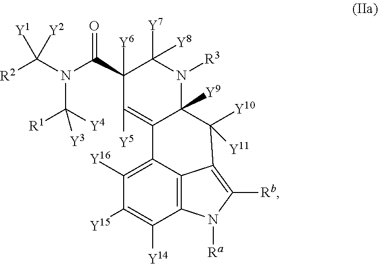
wherein:
R¹, R² and R³ are methyl;
R^{a}, R^{b}, Y¹, Y², Y³, Y⁴, Y⁵, Y⁶, Y⁷, Y⁸, Y⁹, Y¹⁰, Y¹¹, Y¹⁴, Y¹⁵ and Y¹⁶ each are hydrogen or deuterium.

18. The compound of claim 17, wherein at least one of R¹, R², R³, R^{a}, R^{b}, Y, Y², Y³, Y⁴, Y⁵, Y⁶, Y⁷, Y⁸, Y⁹, Y¹⁰, Y¹¹, Y¹⁴, Y¹⁵, and Y¹⁶ is or comprises deuterium.

19. The compound of claim 17 or 18, wherein R¹, R², and R³ are independently selected from CD₃, CD₂H, CDH₂, CT₃, CT₂H, CTH₂ and CH₃.

20. The compound of any one of claims 17-19, wherein R¹, R², and R³ are independently selected from CD₃, CD₂H, CDH₂, and CH₃.

21. The compound of any one of claims 17-20, wherein at least one of R^{a}, R^{b}, Y¹, Y², Y³, Y⁴, Y⁵, Y⁶, Y⁷, Y⁸, Y⁹, Y¹⁰, Y¹¹, Y¹⁴, Y¹⁵, and Y¹⁶ is deuterium.

22. The compound of any one of claims 17-21, wherein the compound is selected from the group consisting of:

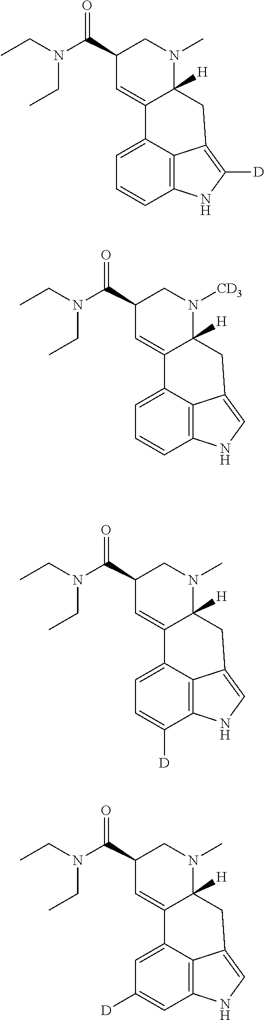 [SMILES: [2H]c1[nH]c2cccc3c2c1C[C@@H]1C3=C[C@@H](C(=O)N(CC)CC)CN1C ; [2H]C([2H])([2H])N1C[C@H](C(=O)N(CC)CC)C=C2c3cccc4[nH]cc(c34)C[C@H]21 ; [2H]c1ccc2c3c(c[nH]c13)C[C@@H]1C2=C[C@@H](C(=O)N(CC)CC)CN1C ; [2H]c1cc2c3c(c[nH]c3c1)C[C@@H]1C2=C[C@@H](C(=O)N(CC)CC)CN1C]

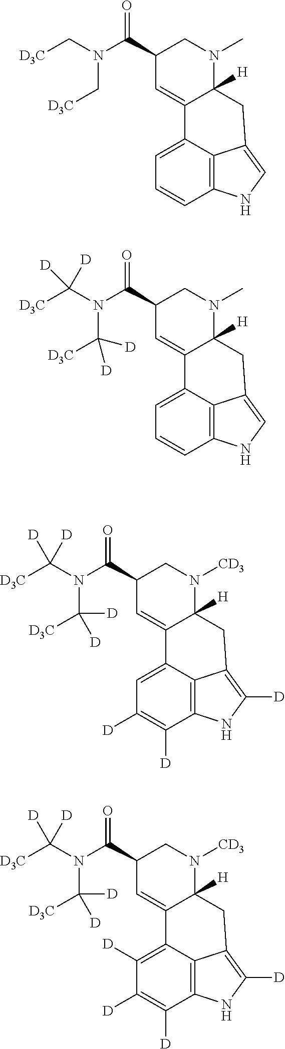 [SMILES: [2H]C([2H])([2H])CN(CC([2H])([2H])[2H])C(=O)[C@@H]1C=C2c3cccc4[nH]cc(c34)C[C@H]2N(C)C1 ; [2H]C([2H])([2H])C([2H])([2H])N(C(=O)[C@@H]1C=C2c3cccc4[nH]cc(c34)C[C@H]2N(C)C1)C([2H])([2H])C([2H])([2H])[2H] ; [2H]c1cc2c3c(c([2H])[nH]c3c1[2H])C[C@@H]1C2=C[C@@H](C(=O)N(C([2H])([2H])C([2H])([2H])[2H])C([2H])([2H])C([2H])([2H])[2H])CN1C([2H])([2H])[2H] ; [2H]c1[nH]c2c([2H])c([2H])c([2H])c3c2c1C[C@@H]1C3=C[C@@H](C(=O)N(C([2H])([2H])C([2H])([2H])[2H])C([2H])([2H])C([2H])([2H])[2H])CN1C([2H])([2H])[2H]]

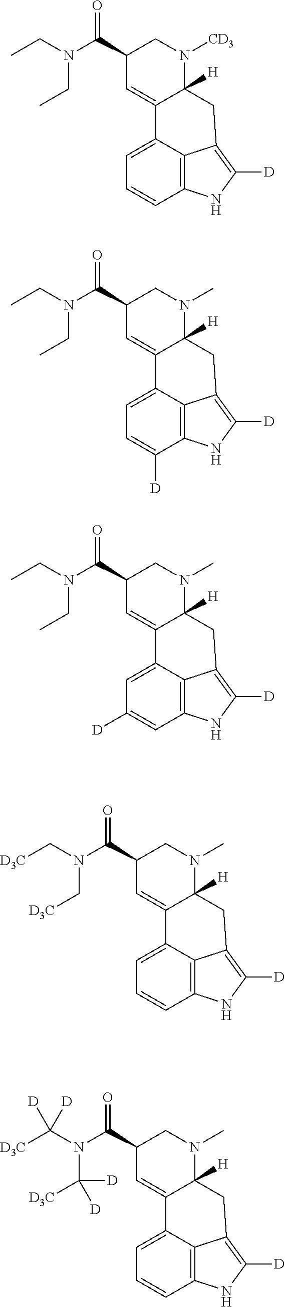 [SMILES: [2H]c1[nH]c2cccc3c2c1C[C@@H]1C3=C[C@@H](C(=O)N(CC)CC)CN1C([2H])([2H])[2H] ; [2H]c1[nH]c2c([2H])ccc3c2c1C[C@@H]1C3=C[C@@H](C(=O)N(CC)CC)CN1C ; [2H]c1cc2c3c(c([2H])[nH]c3c1)C[C@@H]1C2=C[C@@H](C(=O)N(CC)CC)CN1C ; [2H]c1[nH]c2cccc3c2c1C[C@@H]1C3=C[C@@H](C(=O)N(CC([2H])([2H])[2H])CC([2H])([2H])[2H])CN1C ; [2H]c1[nH]c2cccc3c2c1C[C@@H]1C3=C[C@@H](C(=O)N(C([2H])([2H])C([2H])([2H])[2H])C([2H])([2H])C([2H])([2H])[2H])CN1C]

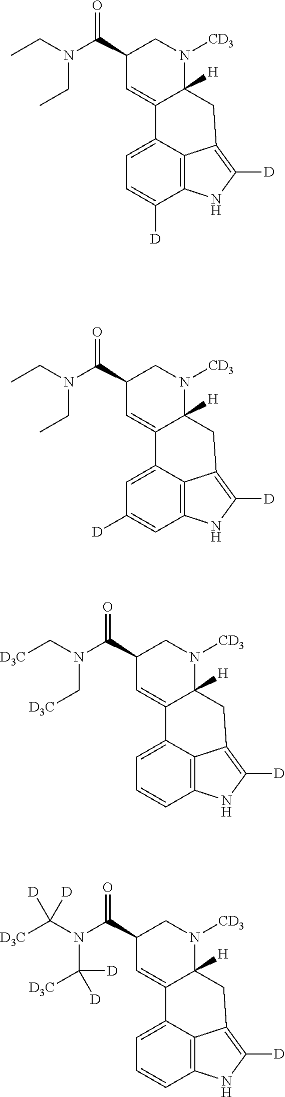 [SMILES: [2H]c1[nH]c2c([2H])ccc3c2c1C[C@@H]1C3=C[C@@H](C(=O)N(CC)CC)CN1C([2H])([2H])[2H] ; [2H]c1cc2c3c(c([2H])[nH]c3c1)C[C@@H]1C2=C[C@@H](C(=O)N(CC)CC)CN1C([2H])([2H])[2H] ; [2H]c1[nH]c2cccc3c2c1C[C@@H]1C3=C[C@@H](C(=O)N(CC([2H])([2H])[2H])CC([2H])([2H])[2H])CN1C([2H])([2H])[2H] ; [2H]c1[nH]c2cccc3c2c1C[C@@H]1C3=C[C@@H](C(=O)N(C([2H])([2H])C([2H])([2H])[2H])C([2H])([2H])C([2H])([2H])[2H])CN1C([2H])([2H])[2H]]

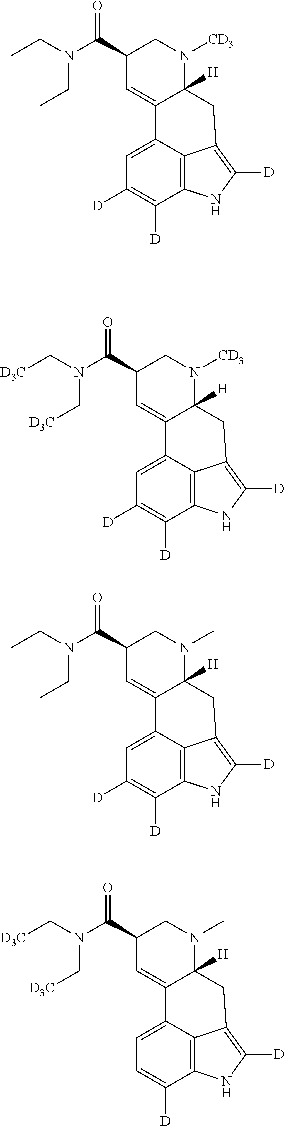 [SMILES: [2H]c1cc2c3c(c([2H])[nH]c3c1[2H])C[C@@H]1C2=C[C@@H](C(=O)N(CC)CC)CN1C([2H])([2H])[2H] ; [2H]c1cc2c3c(c([2H])[nH]c3c1[2H])C[C@@H]1C2=C[C@@H](C(=O)N(CC([2H])([2H])[2H])CC([2H])([2H])[2H])CN1C([2H])([2H])[2H] ; [2H]c1cc2c3c(c([2H])[nH]c3c1[2H])C[C@@H]1C2=C[C@@H](C(=O)N(CC)CC)CN1C ; [2H]c1[nH]c2c([2H])ccc3c2c1C[C@@H]1C3=C[C@@H](C(=O)N(CC([2H])([2H])[2H])CC([2H])([2H])[2H])CN1C]

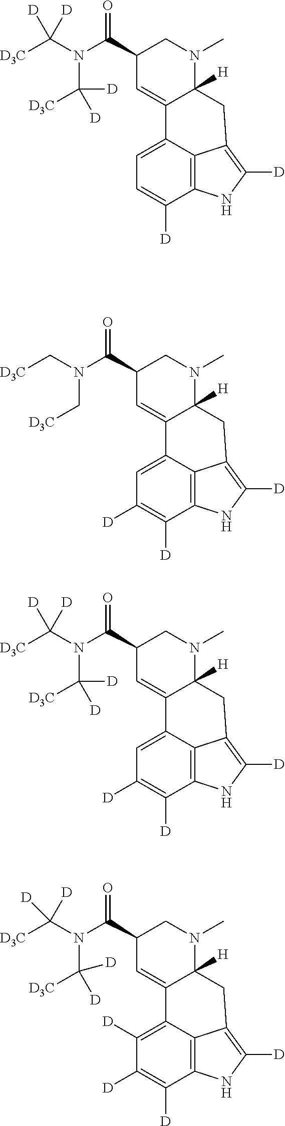 [SMILES: [2H]c1[nH]c2c([2H])ccc3c2c1C[C@@H]1C3=C[C@@H](C(=O)N(C([2H])([2H])C([2H])([2H])[2H])C([2H])([2H])C([2H])([2H])[2H])CN1C ; [2H]c1cc2c3c(c([2H])[nH]c3c1[2H])C[C@@H]1C2=C[C@@H](C(=O)N(CC([2H])([2H])[2H])CC([2H])([2H])[2H])CN1C ; [2H]c1cc2c3c(c([2H])[nH]c3c1[2H])C[C@@H]1C2=C[C@@H](C(=O)N(C([2H])([2H])C([2H])([2H])[2H])C([2H])([2H])C([2H])([2H])[2H])CN1C ; [2H]c1[nH]c2c([2H])c([2H])c([2H])c3c2c1C[C@@H]1C3=C[C@@H](C(=O)N(C([2H])([2H])C([2H])([2H])[2H])C([2H])([2H])C([2H])([2H])[2H])CN1C]

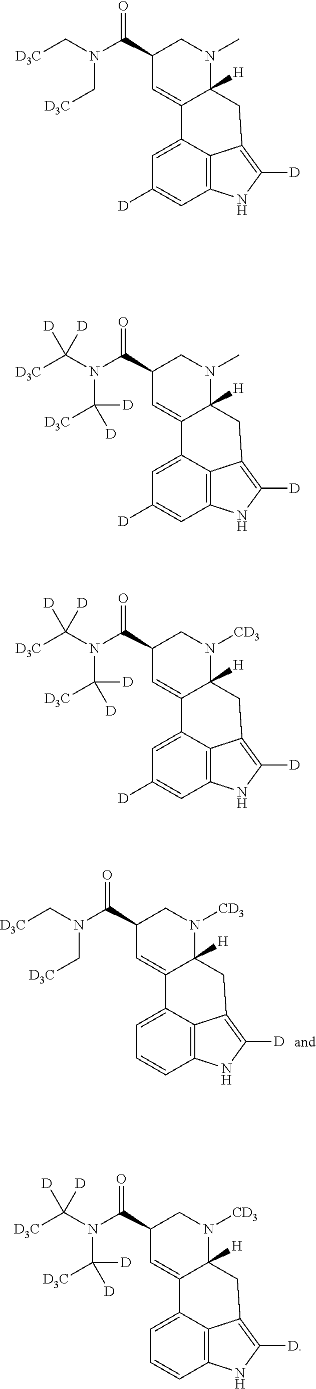

23. The compound of any one of claims 1-22, wherein the compound is of Formula (IIb): 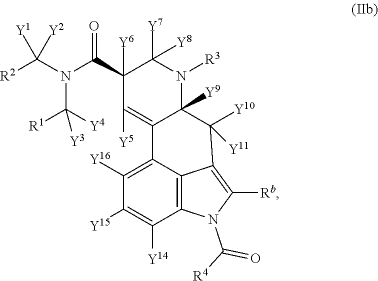
wherein:
R¹, R², and R³ are methyl;
R⁴ is methyl or ethyl; and
R^{b}, Y¹, Y², Y³, Y⁴, Y⁵, Y⁶, Y⁷, Y⁸, Y⁹, Y¹⁰, Y¹¹, Y¹⁴, Y¹⁵ and Y¹⁶ each are hydrogen or deuterium.

24. The compound of claim 23, wherein at least one of R¹, R², R³, R⁴, R^{b}, Y¹, Y², Y³, Y⁴, Y⁵, Y⁶, Y⁷, Y⁸, Y⁹, Y¹⁰, Y¹¹, Y¹⁴, Y¹⁵, and Y¹⁶ is or comprises deuterium.

25. The compound of claim 23 or 24, wherein R¹, R², and R³ are independently selected from CD₃, CD₂H, CDH₂, CT₃, CT₂H, CTH₂ and CH₃.

26. The compound of any one of claims 23-25, wherein R¹, R², and R³ are independently selected from CD₃, CD₂H, CDH₂, and CH₃.

27. The compound of any one of claims 23-26, wherein at least one of R^{b}, Y¹, Y², Y³, Y⁴, Y⁵, Y⁶, Y⁷, Y⁸, Y⁹, Y¹⁰, Y¹¹, Y¹⁴, Y¹⁵, and Y¹⁶ is deuterium.

28. The compound of any one of claims 23-27, wherein the compound is selected from the group consisting of:

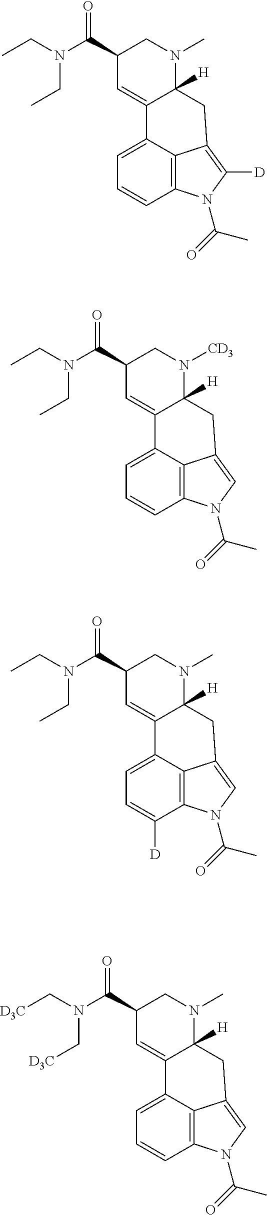

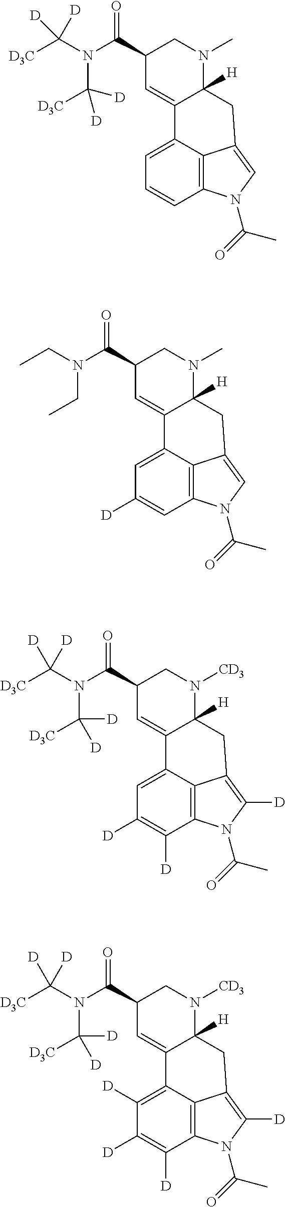

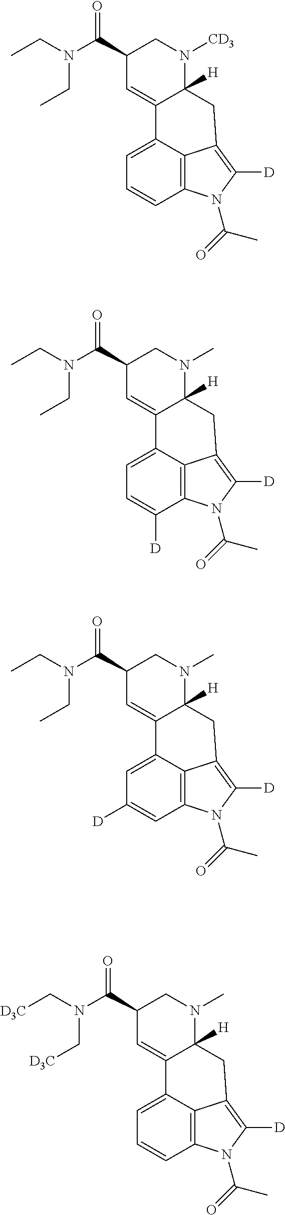 [SMILES: [2H]c1c2c3c(cccc3n1C(C)=O)C1=C[C@@H](C(=O)N(CC)CC)CN(C([2H])([2H])[2H])[C@@H]1C2 ; [2H]c1ccc2c3c(c([2H])n(C(C)=O)c13)C[C@@H]1C2=C[C@@H](C(=O)N(CC)CC)CN1C ; [2H]c1cc2c3c(c([2H])n(C(C)=O)c3c1)C[C@@H]1C2=C[C@@H](C(=O)N(CC)CC)CN1C ; [2H]c1c2c3c(cccc3n1C(C)=O)C1=C[C@@H](C(=O)N(CC([2H])([2H])[2H])CC([2H])([2H])[2H])CN(C)[C@@H]1C2]

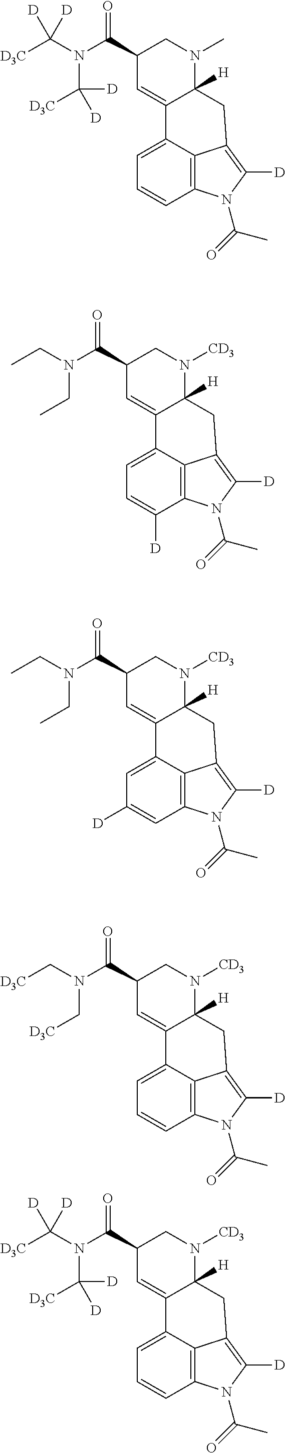

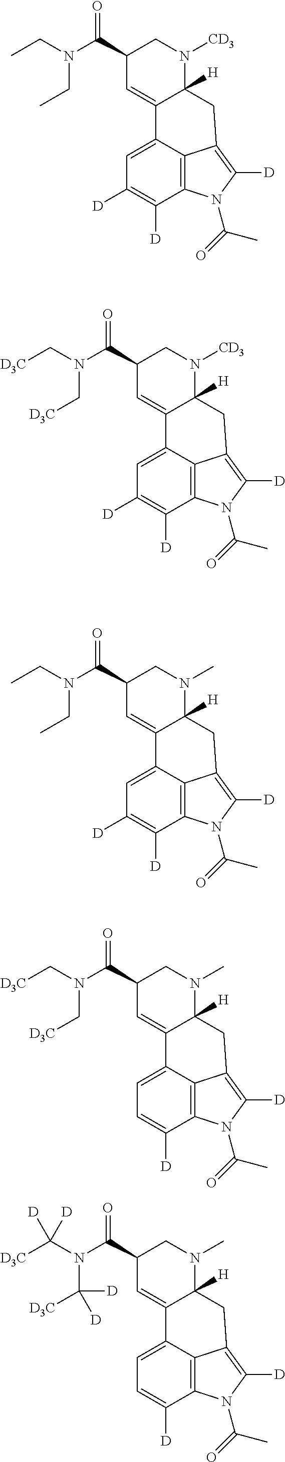

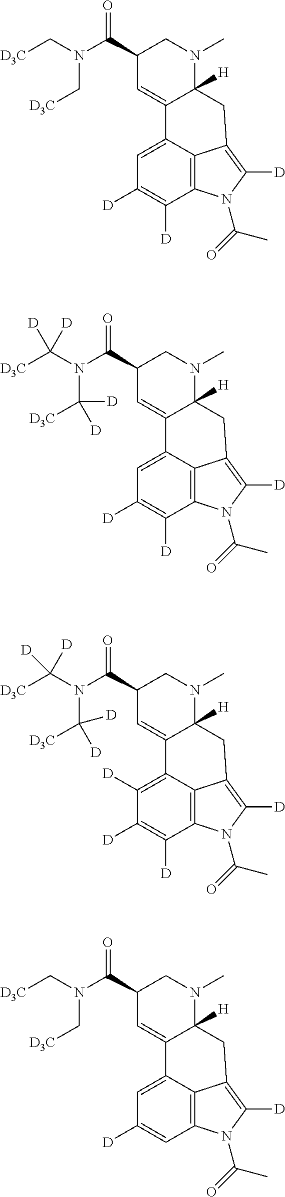 [SMILES: [2H]c1cc2c3c(c([2H])n(C(C)=O)c3c1[2H])C[C@@H]1C2=C[C@@H](C(=O)N(CC([2H])([2H])[2H])CC([2H])([2H])[2H])CN1C ; [2H]c1cc2c3c(c([2H])n(C(C)=O)c3c1[2H])C[C@@H]1C2=C[C@@H](C(=O)N(C([2H])([2H])C([2H])([2H])[2H])C([2H])([2H])C([2H])([2H])[2H])CN1C ; [2H]c1c([2H])c([2H])c2c3c(c([2H])n2C(C)=O)C[C@@H]2C(=C[C@@H](C(=O)N(C([2H])([2H])C([2H])([2H])[2H])C([2H])([2H])C([2H])([2H])[2H])CN2C)c13 ; [2H]c1cc2c3c(c([2H])n(C(C)=O)c3c1)C[C@@H]1C2=C[C@@H](C(=O)N(CC([2H])([2H])[2H])CC([2H])([2H])[2H])CN1C]

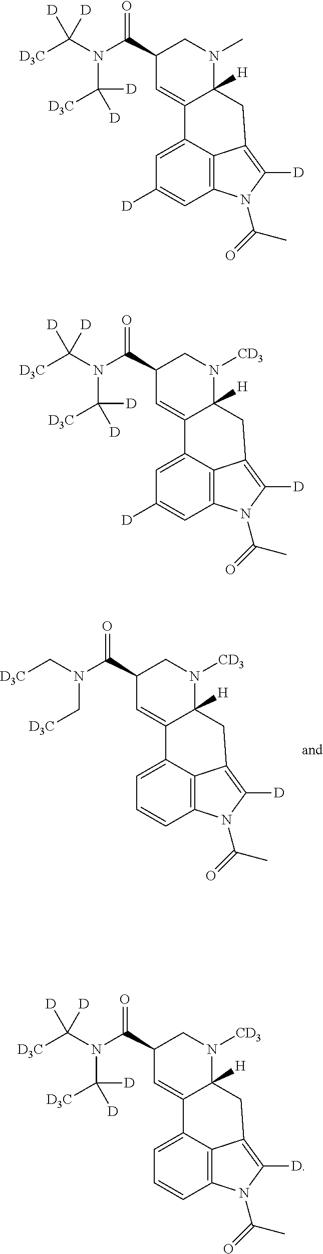

29. The compound of any one of claims 1-28, wherein the compound is of Formula (IIc): 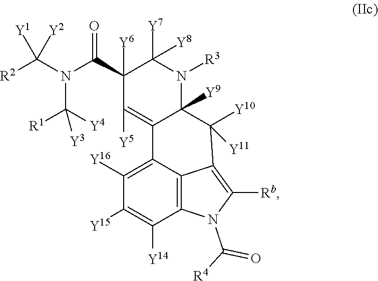
wherein:
R¹, R², and R³ are methyl; and R⁴ is ethyl; and
R^{b}, Y¹, Y², Y³, Y⁴, Y⁵, Y⁶, Y⁷, Y⁸, Y⁹, Y¹⁰, Y¹¹, Y¹⁴, Y¹⁵ and Y¹⁶ each are hydrogen or deuterium.

30. The compound of claim 29, wherein at least one of R¹, R², R³, R⁴, R^{b}, Y¹, Y², Y³, Y⁴, Y⁵, Y⁶, Y⁷, Y⁸, Y⁹, Y¹⁰, Y¹¹, Y¹⁴, Y¹⁵, and Y¹⁶ is or comprises deuterium.

31. The compound of claim 29 or 30, wherein R¹, R², and R³ are independently selected from CD₃, CD₂H, CDH₂, and CH₃.

32. The compound of any one of claims 29-31, wherein R⁴ is CD₂CD₃, CHDCD₃, CH₂CD₃, CH₂CD₃, CD₂CHD₂, CHDCHD₂, CH₂CHD₂, CD₂CH₂D, CHDCH₂D, CH₂CH₂D, CD₂CH₃, CHDCH₃, or CH₂CH₃.

33. The compound of any one of claims 29-32, wherein at least one of R^{b}, Y¹, Y², Y³, Y⁴, Y⁵, Y⁶, Y⁷, Y⁸, Y⁹, Y¹⁰, Y¹¹, Y¹⁴, Y¹⁵, and Y¹⁶ is deuterium.

34. The compound of any one of claims 29-33, wherein the compound is selected from the group consisting of:

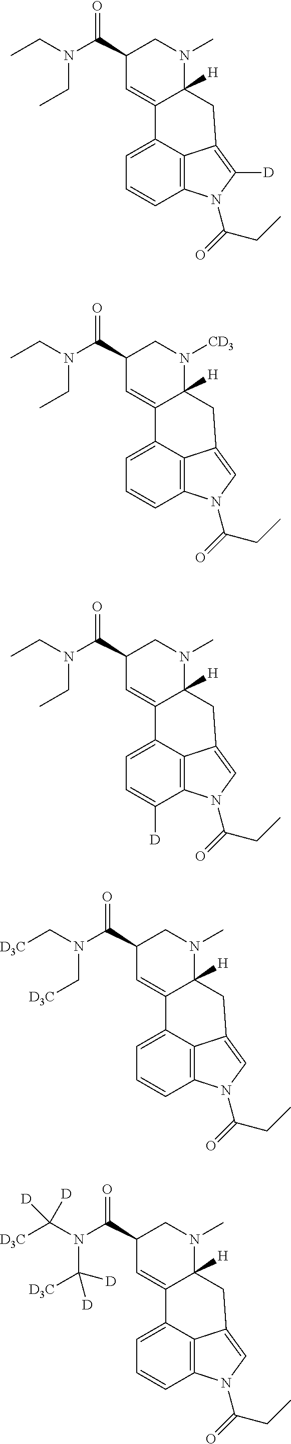

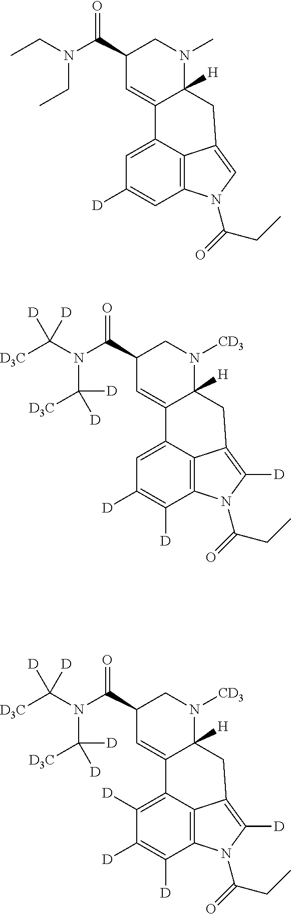 [SMILES: [2H]c1cc2c3c(cn(C(=O)CC)c3c1)C[C@@H]1C2=C[C@@H](C(=O)N(CC)CC)CN1C ; [2H]c1cc2c3c(c([2H])n(C(=O)CC)c3c1[2H])C[C@@H]1C2=C[C@@H](C(=O)N(C([2H])([2H])C([2H])([2H])[2H])C([2H])([2H])C([2H])([2H])[2H])CN1C([2H])([2H])[2H] ; [2H]c1c([2H])c([2H])c2c3c(c([2H])n2C(=O)CC)C[C@@H]2C(=C[C@@H](C(=O)N(C([2H])([2H])C([2H])([2H])[2H])C([2H])([2H])C([2H])([2H])[2H])CN2C([2H])([2H])[2H])c13]

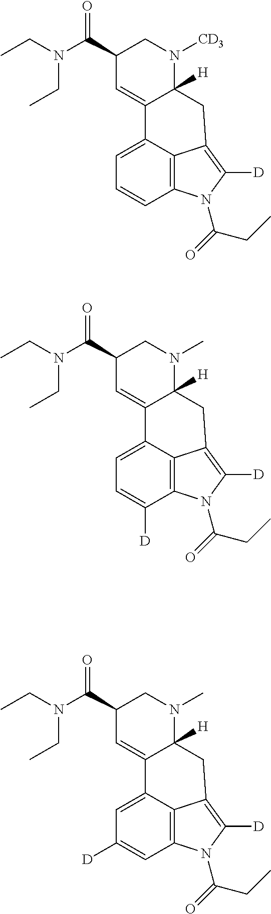

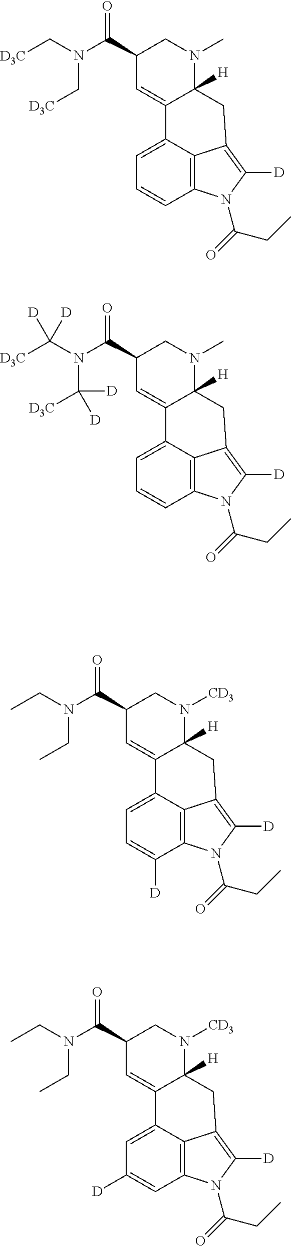

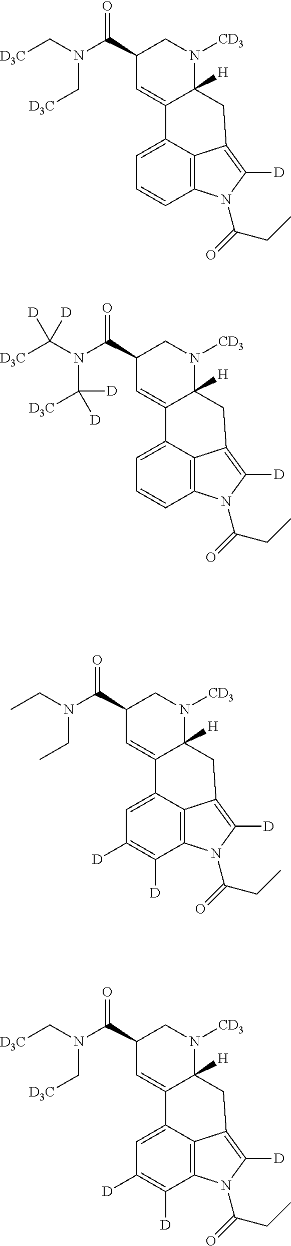

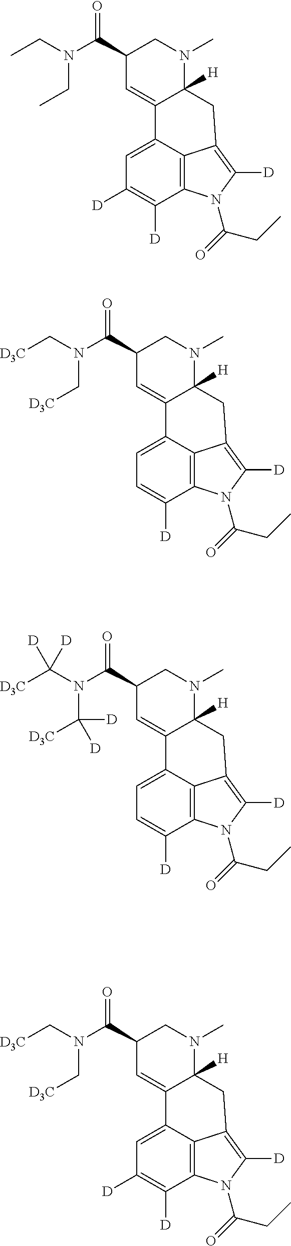 [SMILES: [2H]c1cc2c3c(c([2H])n(C(=O)CC)c3c1[2H])C[C@@H]1C2=C[C@@H](C(=O)N(CC)CC)CN1C ; [2H]c1ccc2c3c(c([2H])n(C(=O)CC)c13)C[C@@H]1C2=C[C@@H](C(=O)N(CC([2H])([2H])[2H])CC([2H])([2H])[2H])CN1C ; [2H]c1ccc2c3c(c([2H])n(C(=O)CC)c13)C[C@@H]1C2=C[C@@H](C(=O)N(C([2H])([2H])C([2H])([2H])[2H])C([2H])([2H])C([2H])([2H])[2H])CN1C ; [2H]c1cc2c3c(c([2H])n(C(=O)CC)c3c1[2H])C[C@@H]1C2=C[C@@H](C(=O)N(CC([2H])([2H])[2H])CC([2H])([2H])[2H])CN1C]

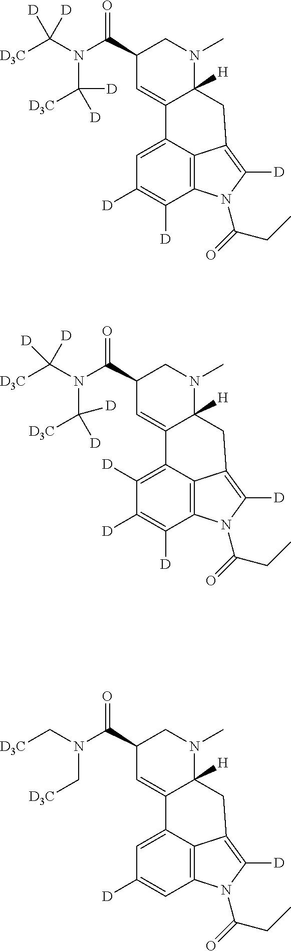 [SMILES: [2H]c1cc2c3c(c([2H])n(C(=O)CC)c3c1[2H])C[C@@H]1C2=C[C@@H](C(=O)N(C([2H])([2H])C([2H])([2H])[2H])C([2H])([2H])C([2H])([2H])[2H])CN1C ; [2H]c1c([2H])c([2H])c2c3c(c([2H])n2C(=O)CC)C[C@@H]2C(=C[C@@H](C(=O)N(C([2H])([2H])C([2H])([2H])[2H])C([2H])([2H])C([2H])([2H])[2H])CN2C)c13 ; [2H]c1cc2c3c(c([2H])n(C(=O)CC)c3c1)C[C@@H]1C2=C[C@@H](C(=O)N(CC([2H])([2H])[2H])CC([2H])([2H])[2H])CN1C]

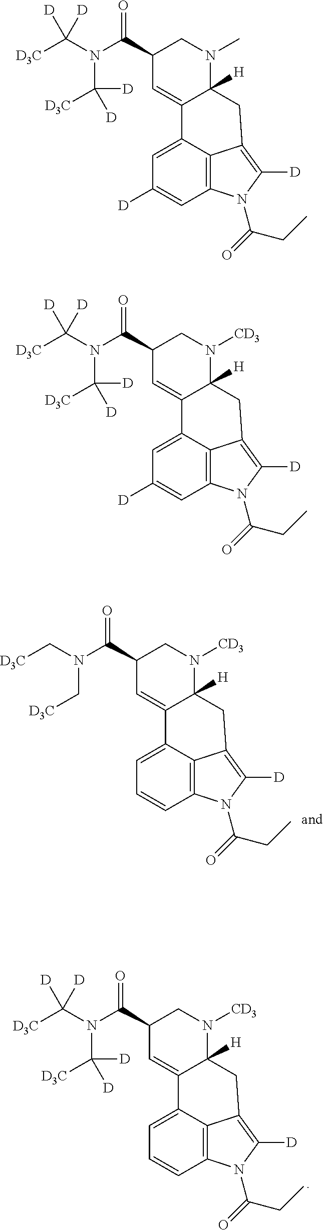

35. The compound of any one of claims 1-34, wherein:
R^{b} is bromo;
Y¹⁴, Y¹⁵, and Y¹⁶ are deuterium;
R¹, R², and R³ are independently selected from CD₃, CD₂H, CDH₂, and CH₃; and
R^{a}, Y¹, Y², Y³, Y⁴, Y⁵, Y⁶, Y⁷, Y⁸, Y⁹, Y¹⁰, and Y¹¹ are independently hydrogen or deuterium.

36. The compound of any one of claims 1-34, wherein:
R^{b} is bromo;
R³ is CD₃;
R¹ and R² are independently selected from CD₃, CD₂H, CDH₂, and CH₃; and
R^{a}, Y¹, Y², Y³, Y⁴, Y⁵, Y⁶, Y⁷, Y⁸, Y⁹, Y¹⁰, Y¹¹, Y¹⁴, Y¹⁵, and Y¹⁶ are independently hydrogen or deuterium.

37. The compound of any one of claims 1-34, wherein:
R^{b} is bromo;
R¹ and R² are CD₃;
Y¹, Y², Y³, and Y⁴ are deuterium;
R³ is selected from CD₃, CD₂H, CDH₂, and CH₃; and
R^{a}, Y⁵, Y⁶, Y⁷, Y⁸, Y⁹, Y¹⁰, Y¹¹, Y¹⁴, Y¹⁵, and Y¹⁶ are independently hydrogen or deuterium.

38. The compound of any one of claims 1-34, wherein:
R^{b} is bromo;
R¹, R², and R³ are CD₃;
Y¹, Y², Y³, and Y⁴ are deuterium; and
R^{a}, Y⁵, Y⁶, Y⁷, Y⁸, Y⁹, Y¹⁰, Y¹¹, Y¹⁴, Y¹⁵, and Y¹⁶ are independently hydrogen or deuterium.

39. The compound of any one of claims 1-34, wherein:
R¹ and R² are CD₃;
Y¹, Y², Y³, and Y⁴ are deuterium;
R³ is selected from CD₃, CD₂H, CDH₂, and CH₃; and
R^{a}, R^{b}, Y⁵, Y⁶, Y⁷, Y⁸, Y⁹, Y¹⁰, Y¹¹, Y¹⁴, Y¹⁵, and Y¹⁶ are independently hydrogen or deuterium.

40. The compound of any one of claims 1-34, wherein:
R¹ and R² are CD₃;
R^{b}, Y¹, Y², Y³, Y⁴, Y¹⁴, Y¹⁵, and Y¹⁶ are deuterium;
R³ is selected from CD₃, CD₂H, CDH₂, and CH₃; and
R^{a}, Y⁵, Y⁶, Y⁷, Y⁸, Y⁹, Y¹⁰, and Y¹¹ are independently hydrogen or deuterium.

41. The compound of any one of claims 1-34, wherein:
R¹ and R² are CD₃;
R^{b}, Y¹, Y², Y³, and Y⁴ are deuterium;
R³ is selected from CD₃, CD₂H, CDH₂, and CH₃; and
R^{a}, Y⁵, Y⁶, Y⁷, Y⁸, Y⁹, Y¹⁰, Y¹¹, Y¹⁴, Y¹⁵, and Y¹⁶ are independently hydrogen or deuterium.

42. The compound of any one of claims 1-34, wherein:
R¹, R², and R³ are CD₃;
Y¹, Y², Y³, and Y⁴ are deuterium; and
R^{a}, R^{b}, Y⁵, Y⁶, Y⁷, Y⁸, Y⁹, Y¹⁰, Y¹¹, Y¹⁴, Y¹⁵, and Y¹⁶ are independently hydrogen or deuterium.

43. The compound of any one of claims 1-34, wherein:
R^{b}, Y¹⁴, Y¹⁵, and Y¹⁶ are deuterium;
R¹, R², and R³ are independently selected from CD₃, CD₂H, CDH₂, and CH₃; and
R^{a}, Y¹, Y², Y³, Y⁴, Y⁵, Y⁶, Y⁷, Y⁸, Y⁹, Y¹⁰, and Y¹¹ are independently hydrogen or deuterium.

44. The compound of any one of claims 1-34, wherein:
R^{b} is deuterium;
R¹, R², and R³ are independently selected from CD₃, CD₂H, CDH₂, and CH₃; and
R^{a}, Y¹, Y², Y³, Y⁴, Y⁵, Y⁶, Y⁷, Y⁸, Y⁹, Y¹⁰, Y¹¹, Y¹⁴, Y¹⁵, and Y¹⁶ are independently hydrogen or deuterium.

45. The compound of any one of claims 1-34, wherein:
R³ is CD₃;
R¹ and R² are independently selected from CD₃, CD₂H, CDH₂, and CH₃; and
R^{a}, R, Y¹, Y², Y³, Y⁴, Y⁵, Y⁶, Y⁷, Y⁸, Y⁹, Y¹⁰, Y¹¹, Y¹⁴, Y¹⁵, and Y¹⁶ are independently hydrogen or deuterium.

46. A compound having any one of the structures shown in Table 1, or a pharmaceutically acceptable salt thereof.

47. The compound of any one of claims 1-46, wherein the compound is in the form of a pharmaceutically acceptable salt.

48. The compound of any one of claims 1-47, wherein the compound is in the form of a solvate.

49. A pharmaceutical composition comprising a compound of any one of claims 1-48.

50. A method for increasing neuronal plasticity, comprising contacting a neuron with an effective amount of a compound according to any one of claims 1-48.

51. The method of claim 50, wherein contacting comprises administering the compound to a subject.

52. A method for treating a neurological disorder, a psychiatric disorder, or both, comprising contacting a subject having the neurological disorder, psychiatric disorder or both with an effective amount of a compound according to any one of claims 1-48.

53. The method of claim 52, wherein the neurological disorder is a neurodegenerative disorder.

54. The method of claim 52, wherein the neurological disorder or psychiatric disorder, or both, comprises depression, addiction, anxiety, or a post-traumatic stress disorder.

55. The method of claim 52, wherein the neurological disorder, psychiatric disorder, or both, comprises treatment resistant depression, suicidal ideation, major depressive disorder, bipolar disorder, schizophrenia, or substance use disorder.

56. The method of claim 52, wherein the neurological disorder, psychiatric disorder, or both, comprises stroke, traumatic brain injury, or a combination thereof.

57. The method of claim 52, further comprising administering to the subject an effective amount of an empathogenic agent.

58. The method of claim 57, wherein the empathogenic agent is MDMA.

59. The method of claim 52, further comprising administering a 5-HT_{2A} antagonist to the subject.

60. The method of claim 59, wherein the 5-HT_{2A} antagonist is selected from MDL-11,939, eplivanserin (SR-46,349), ketanserin, ritanserin, altanserin, acepromazine, mianserin, mirtazapine, quetiapine, SB204741, SB206553, SB242084, LY272015, SB243213, blonanserin, SB200646, RS102221, nefazodone, volinanserin (MDL-100,907), olanzapine, risperidone, pimavanserin, nelotanserin and lorcaserin.
